(19) United States
(12) Patent Application Publication
(10) Pub. No.: US 2023/0293603 A1
(43) Pub. Date: Sep. 21, 2023

(54) RECOMBINANT BACTERIA FOR PRODUCTION OF D-LACTATE AND/OR L-LACTATE AND USES THEREOF

(71) Applicants: Synlogic Operating Company, Inc., Cambridge, CA (US); The Brigham and Women's Hospital, Inc., Boston, MA (US)

(72) Inventors: Ning Li, Winchester, MA (US); Anna Sokolovska, Somerville, MA (US); Francisco Quintana, Boston, MA (US); Liliana Sanmarco, Boston, MA (US)

(21) Appl. No.: 18/019,383

(22) PCT Filed: Aug. 6, 2021
(86) PCT No.: PCT/US21/44923

§ 371 Date: Feb. 2, 2023

Related U.S. Application Data
(60) Provisional application No. 63/062,154, filed on Aug. 6, 2020.

(51) Int. Cl. A61K 35/745; C12N 9/10; C12N 9/12; C12N 9/04; C12P 7/56; A61K 35/742; A61K 35/741; A61K 35/747; A61K 35/744

(52) U.S. Cl. CPC A61K 35/745; C12N 9/1029; C12Y 203/01008; C12Y 203/01054; C12N 9/1217; C12Y 207/02001; C12N 9/0006; C12Y 101/01027; C12P 7/56; A61K 35/742; A61K 35/741; A61K 35/747; A61K 35/744

(57) ABSTRACT

The present disclosure provides recombinant bacteria for production of D-lactate and/or L-lactate. Pharmaceutical compositions and methods of treating diseases are also included in the present invention.

### RELATED APPLICATIONS

This application claims priority to U.S. Provisional Application No. 63/062,154, filed on Aug. 6, 2020, the entire contents of which are expressly incorporated by reference herein in their entirety.

### SEQUENCE LISTING

The instant application contains a Sequence Listing which has been submitted electronically in ASCII format and is hereby incorporated by reference in its entirety. Said ASCII copy, created on Aug. 6, 2021, is named 126046-05920_SL.txt and is 111,795 bytes in size.

### BACKGROUND

Dendritic cells (DCs) control T-cell activation and therefore, DC activation and function represent potential therapeutic targets to control inflammation, such as in autoimmune and inflammatory disease and disorders. Currently, there are limited treatments available to treat autoimmune and inflammatory disease and disorders, e.g., multiple sclerosis and inflammatory brain disease. Accordingly, there exists an ongoing need for novel compositions for treating and/or preventing autoimmune and inflammatory disease or disorders.

### SUMMARY

The present disclosure provides a recombinant bacteria for production of D-lactate and/or L-lactate, pharmaceutical compositions thereof, and methods of modulating and treating diseases, such as autoimmune and inflammatory disease. The recombinant bacteria are capable of producing D-lactate and/or L-lactate in low-oxygen environments, e.g., the gut. Thus, the recombinant bacteria and pharmaceutical compositions comprising those bacteria are non-pathogenic, and can be used in order to treat and/or prevent conditions associated with diseases, including autoimmune and inflammatory diseases and disorders.

This disclosure provides, in one aspect, a recombinant bacterium comprising an ldhA gene for producing D-lactate, wherein the ldhA gene is operably linked to a directly or indirectly inducible promoter that is not associated with the ldhA gene in nature, and wherein the promoter is induced by exogenous environmental conditions. In one embodiment, the ldhA gene is a heterologous gene.

In some embodiments, the recombinant bacteria further comprises a deletion or mutation in one or more gene(s) selected from the group comprising formate acetyltransferase 1 (pf1B), acetate kinase (ackA), methylglyoxyl synthetase (mgsA), fumarase reductase subunit (frdB), fumarase reductase subunit (frdC), aldehyde dehydrogenase (adhE), phosphofructokinase (pfkA), and/or phosphate acetyltransferase (pta). In some embodiments, the recombinant bacteria comprises a deletion or mutation is in the pta gene. In some embodiments, the recombinant bacteria comprises a deletion or mutation is in a ackA gene. In some embodiments, the recombinant bacteria comprises a deletion or mutation is in a pflB gene. In some embodiments, the recombinant bacteria comprises a deletion or mutation is in a mgsA gene. In some embodiments, the recombinant bacteria comprises a deletion or mutation is in a frdB gene. In some embodiments, the recombinant bacteria comprises a deletion or mutation is in a frdC gene. In some embodiments, the recombinant bacterium may comprise a mutation or a deletion in a adhE gene. In some embodiments, the recombinant bacterium may comprise a mutation or deletion in a pfkA gene. In some embodiments, the recombinant bacteria further comprises a ribosome binding site before ldhA gene.

In some embodiments, the recombinant bacteria comprises a promoter is directly or indirectly induced by low-oxygen or anaerobic conditions. In some embodiments, the promoter is an FNR-inducible promoter. In one embodiment, the promoter has at least 90%, 91%, 92%, 93%, 94%, 95%, 96%, 97%, 98%, 99%, comprises or consists of SEQ ID NO:27.

In some embodiments, the recombinant bacteria wherein the one or more gene cassettes are operably linked to a temperature-sensitive promoter. In some embodiments, the temperature-sensitive promoter is cI857. In one embodiment, the promoter has at least 90%, 91%, 92%, 93%, 94%, 95%, 96%, 97%, 98%, 99%, comprises or consists of SEQ ID NO:28.

In some embodiments, the one or more gene cassettes and operatively linked promoter are present on a plasmid in the bacterium. In some embodiments, the one or more gene cassettes and operatively linked promoter are present on a chromosome in the bacterium.

In some embodiments, the bacterium is a non-pathogenic bacterium. In some embodiments, the bacterium is a probiotic or a commensal bacterium.

In some embodiments, the bacterium is selected from the group consisting of Bacteroides, Bifidobacterium, Clostridium, Escherichia, Lactobacillus, and Lactococcus. In some embodiments, the bacterium is Escherichia coli strain Nissle.

In some embodiments, the bacterium is capable of producing about 1 mM D-lactate to about 20 mM D-lactate. In some embodiments, the bacterium is capable of producing about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM or about 20 mM D-lactate. In some embodiments, the bacterium is capable of producing about 1-20 mM, about 2-20 mM, about 3-20 mM, about 4-20 mM, about 5-20 mM, about 10-20 mM, about 15-20 mM, about 1-15 mM, about 2-15 mM, about 3-15 mM, about 4-15 mM, about 5-10 mM, about 10-15 mM, about 1-10 mM, about 2-10 mM, about 3-10 mM, about 4-10 mM, or about 5-10 mM D-lactate.

In some embodiments, the bacterium is capable of producing about 1 μmol/10⁹ cells/hour, 2 μmol/10⁹ cells/hour, or 3 μmol/10⁹ cells/hour D-lactate in vitro. In some embodiments, the bacterium us capable of producing 2 μmol/10⁹ cells/hour D-lactate in vitro. In some embodiments, the bacterium us capable of producing about 1 to about 3 μmol/10⁹ cells/hour D-lactate in vitro.

In another aspect, the disclosure provides a pharmaceutically acceptable composition comprising the bacterium as described herein; and a pharmaceutically acceptable carrier.

In some embodiments, the pharmaceutically acceptable composition is formulated for oral administration.

In one aspect, the invention provides a method of treating a disease or disorder in a subject in need thereof. The method comprises the step of administering to the subject the pharmaceutical composition as described herein.

In one embodiment, the disease or disorder is a an autoimmune disease or inflammatory disease or disorder. In one embodiment, the disease or disorder is selected from the group consisting of multiple sclerosis, central nervous system inflammation (CNS) inflammation, 2,4,6-trinitrobenzene sulfonic acid (TNBS)-induced colitis, T cell-induced colitis, T cell-induced small bowel inflammation, chronic colitis, rheumatoid arthritis, celiac disease, myasthenia gravis, and B-cell-mediated T-cell-dependent autoimmune disease.

In one aspect, the disclosure provides a method of treating, reducing, or ameliorating symptoms of a disease or disorder in a subject in need thereof. The method comprises the step of administering to the subject the pharmaceutical composition as described herein. In one embodiment, the symptom of the disease or disorder is inflammation.

In some embodiments, the subject has an increased level of D-lactate after the composition is administrated. In some embodiments, the subject is a human.

Mammalian cells contain only L-lactate and, therefore, in humans the lactate produced is almost exclusively L-lactate. Therefore, after administration of the recombinant bacteria disclosed herein to a human subject, production of D-lactate in the urine of the human subject can serve as marker for therapeutic efficacy. Accordingly, disclosed herein is a method comprising (a) measuring a level of D-lactate in the urine of a subject at a first time point prior to administration of a recombinant bacterium disclosed herein; (b) measuring a level of D-lactate in the urine of the subject at a second time point after administration of the recombinant bacterium. In some embodiments, an increase of D-lactate in the urine in the subject at the second time point as compared to the first time point indicates that the treatment is efficacious.

In some embodiments, the administration of the pharmaceutical composition represses effector T cells by at least 1.5 fold, at least 1.8-fold, at least 2-fold, at least 2.2-fold, or at least 2.5-fold when compared to a control, wherein the control has not been treated with the pharmaceutical composition. In some embodiments, the effector T cells are repressed by at least 2-fold when compared to the control.

In some embodiments, the effector T cells are IFNγ⁺/CD4 T cells and/or IFN-γ⁺/IL-17⁺/CD4 T cells.

In some embodiments, the administration of the pharmaceutical composition increases expression of Hypoxia-inducible factor 1-alpha (HIF-1α) in dendritic cells by at least 1.5 fold, at least 1.8-fold, at least 2-fold, at least 2.2-fold, at least 2.5-fold, or at least 3-fold when compared to a control, wherein the control has not been treated with the pharmaceutical composition. In some embodiments, the e expression of HIF-1α is increased by at least 2-fold when compared to the control.

In some embodiments, the administration of the pharmaceutical composition decreases re-stimulation of T cells by at least 1.5 fold, at least 1.8-fold, at least 2-fold, at least 2.2-fold, or at least 2.5-fold when compared to a control, wherein the control has not been treated with the pharmaceutical composition.

In some embodiments, the administration of the pharmaceutical composition decreases expression of an inflammatory cytokine(s) by at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, and least 2.9-fold, or at least 3.0-fold when compared to a control. In one embodiment, the control has not been administered the pharmaceutical composition. In some embodiments, the inflammatory cytokine(s) is IL-17A, IL-10, and/or IFN-γ.

Some bacterial species produce only D-lactate or L-lactate. Carbohydrate-fermenting bacterial species, such as Lactobacillus (L. acidophilus, L. gasseri, L. delbrueckii subsp. Bulgaricus, L. fermentum, L. lactis, L. brevis, L. helveticus, L. plantarum and L. reuteri) have both enzymes and the capacity to produce both L-lactate and D-lactate.

In another aspect, the disclosure provides a recombinant bacterium comprising an ldhL gene for producing L-lactate, wherein the ldhL gene is operably linked to a directly or indirectly inducible promoter that is not associated with the ldhL gene in nature, and wherein the promoter is induced by exogenous environmental conditions. In one embodiment, the ldhL gene is a heterologous gene.

In some embodiments, the recombinant bacteria further comprises a deletion or mutation in a gene selected from the group comprising formate acetyltransferase 1 (pflB), acetate kinase (ackA), methylglyoxyl synthetase (mgsA), fumarase reductase subunit (frdB), fumarase reductase subunit (frdC), aldehyde dehydrogenase (adhE), phosphofructokinase (pfkA) and/or phosphate acetyltransferase (pta). In some embodiments, the recombinant bacteria comprises a deletion or mutation is in a pta gene. In some embodiments, the recombinant bacteria comprises a deletion or mutation is in an ackA gene. In some embodiments, the recombinant bacteria comprises a deletion or mutation is in a pflB gene. In some embodiments, the recombinant bacteria comprises a deletion or mutation is in an msgA gene. In some embodiments, the recombinant bacteria comprises a deletion or mutation is in aft-dB gene. In some embodiments, the recombinant bacteria comprises a deletion or mutation is in an frdC gene. In some embodiments, the recombinant bacterium may comprise a mutation or a deletion in an adhE gene. In some embodiments, the recombinant bacterium may comprise a mutation or deletion in a pfkA gene.

In some embodiments, the recombinant bacteria further comprises a ribosome binding site before ldhL gene.

In some embodiments, the promoter is directly or indirectly induced by low-oxygen or anaerobic conditions. In some embodiments, the promoter is an FNR-inducible promoter. In one embodiment, the promoter has at least 90%, 91%, 92%, 93%, 94%, 95%, 96%, 97%, 98%, 99%, comprises or consists of SEQ ID NO:27.

In some embodiments, the promoter is a temperature-sensitive promoter. In some embodiments, the temperature-sensitive promoter is cI857. In one embodiment, the promoter has at least 90%, 91%, 92%, 93%, 94%, 95%, 96%, 97%, 98%, 99%, comprises or consists of SEQ ID NO:28.

In some embodiments, the ldhL gene and operatively linked promoter are present on a plasmid in the bacterium. In some embodiments, the ldhL gene and operatively linked promoter are present on a chromosome in the bacterium.

In some embodiments, the bacterium is a non-pathogenic bacterium. In some embodiments, the bacterium is a probiotic or a commensal bacterium. In some embodiments, the bacterium is selected from the group consisting of Bacteroides, Bifidobacterium, Clostridium, Escherichia, Lactobacillus, and Lactococcus. In some embodiments, the bacterium is Escherichia coli strain Nissle.

In another aspect, the disclosure provides a pharmaceutically acceptable composition comprising a bacterium as described herein; and a pharmaceutically acceptable carrier. In some embodiments, the pharmaceutically acceptable composition is formulated for oral administration.

In one aspect, the disclosure provides a method of treating a disease or disorder in a subject in need thereof. The method comprises the step of administering to the subject the pharmaceutical composition as described herein.

In one embodiment, the disease or disorder is a an autoimmune disease or inflammatory disease or disorder. In one embodiment, the disease or disorder is selected from the group consisting of multiple sclerosis, central nervous system inflammation (CNS) inflammation, 2,4,6-trinitrobenzene sulfonic acid (TNBS)-induced colitis, T cell-induced colitis, T cell-induced small bowel inflammation, chronic colitis, rheumatoid arthritis, celiac disease, myasthenia gravis, and B-cell-mediated T-cell-dependent autoimmune disease.

In one aspect, the disclosure provides a method of treating, reducing, or ameliorating symptoms of a disease or disorder in a subject in need thereof. The method comprises the step of administering to the subject the pharmaceutical composition as described herein. In one embodiment, the symptom of the disease or disorder is inflammation.

In some embodiments, the subject has an increased level of L-lactate after the composition is administrated. In some embodiments, the subject is a human.

In some embodiments, the administration of the pharmaceutical composition represses effector T cells by at least 1.5 fold, at least 1.8-fold, at least 2-fold, at least 2.2-fold, or at least 2.5-fold when compared to a control, wherein the control has not been treated with the pharmaceutical composition. In some embodiments, the effector T cells are repressed by at least 2-fold when compared to the control.

In some embodiments, the effector T cells are IFN-γ⁺/CD4 T cells and/or IFN-γ⁺/IL-17⁺/CD4 T cells.

In some embodiments, the administration of the pharmaceutical composition increases expression of Hypoxia-inducible factor 1-alpha (HIF-1α) in dendritic cells by at least 1.5 fold, at least 1.8-fold, at least 2-fold, at least 2.2-fold, at least 2.5-fold, or at least 3-fold when compared to a control, wherein the control has not been treated with the pharmaceutical composition. In some embodiments, the e expression of HIF-1α is increased by at least 2-fold when compared to the control.

In some embodiments, the administration of the pharmaceutical composition decreases re-stimulation of T cells by at least 1.5 fold, at least 1.8-fold, at least 2-fold, at least 2.2-fold, or at least 2.5-fold when compared to a control, wherein the control has not been treated with the pharmaceutical composition.

In one aspect, disclosed herein is a method of activating the G-protein coupled receptor (GPR81), the method comprising administering a pharmaceutical composition comprising a bacterium described herein to a subject, thereby activating GPR81. In one embodiment, activation of GPR81 treats inflammation, e.g., colonic inflammation, in the subject. In one embodiment, activation of GPR81 prevents inflammation, e.g., colonic inflammation, in the subject.

### BRIEF DESCRIPTION OF THE FIGURES

FIG. 1A depicts a metabolic pathway for D-lactate production.

FIG. 1B depicts a metabolic pathway for L-lactate production.

FIG. 1C depicts a schematic of a recombinant bacterium that is genetically engineered to express a D-lactate biosynthesis gene, ldhA, which produces D-lactate from pyruvate. The ldhA gene is under the control of a FNR-responsive or temperature sensitive promoter. The genetically engineered bacterium, e.g., E. coli further comprises a deletion in the pta gene.

FIG. 2A depicts a D-lactate kit standard curve from D-lactate detection using a fluorometric D-lactate assay kit (duplicate) of strains used in FIG. 2B.

FIG. 2B depicts a graph of D-lactate production using the strains SYN094, control; SYN6527, Δpta; SYN6528, Δpta, pSC101-cI857ldhA-carb; and SYN6529, Δpta, pSC101-fnr-ldhA-carb.

FIG. 2C depicts a D-lactate kit standard curve from D-lactate detection using a fluorometric D-lactate assay kit (duplicate) of strains used in FIG. 2D.

FIG. 2D depicts a graph of D-lactate production using the strains SYN6524, ΔadhE; SYN6525, ΔadhE, pSC101-cI857ldhA-carb; SYN6526, ΔadhE, pSC101-fnr-ldhA-carb; SYN6527, Δpta; SYN6528, Δpta, pSC101-cI857ldhA-carb; SYN6529, Δpta, pSC101-fnr-ldhA-carb; SYN6265, ΔpfkA-Kan; SYN6530, ΔpfkA-Kan, pSC101-cI857ldhA-carb; SYN6531, SYN001, ΔpfkA-Kan, pSC101-fnr-ldhA-carb; SYN094, SYN001 strpR, control; SYN6522, SYN001, strpR, pSC101-cI857-ldhA-carb; and SYN6523, SYN001 strpR, pSC101-fnr-ldhA-carb.

FIG. 3A depicts the engineered bacterial strain producing D-Lactate (SYN6528) in the mouse gut suppresses neuroinflammation and ameliorates development of experimental autoimmune encephalomyelitis (EAE). Control Bact: SYN094; D-LA Bact: SYN6528; vehicle.

FIG. 3B depicts SYN6528 in the mouse gut decreases the number of pathogenic effector T cells in the mouse brain. Control Bact: SYN094; D-LA Bact: SYN6528; vehicle.

FIG. 4A depicts SYN6528 increased HIF-1α expression in dendritic cells (DCs) leading to immunoregulation and control of T cell compartment. Increased percentage of anti-inflammatory HIF-1α-positive DCs after treatment with SYN6528. Control Bact: SYN094; D-LA Bact: SYN6528; vehicle.

FIG. 4B depicts SYN6528 lowered recall response to MOG35-55 (EAE antigen) re-stimulation in splenocytes (T cells) from D-Lactate Bacteria treated mice. Control Bact: SYN094; D-LA B act: SYN6528; vehicle.

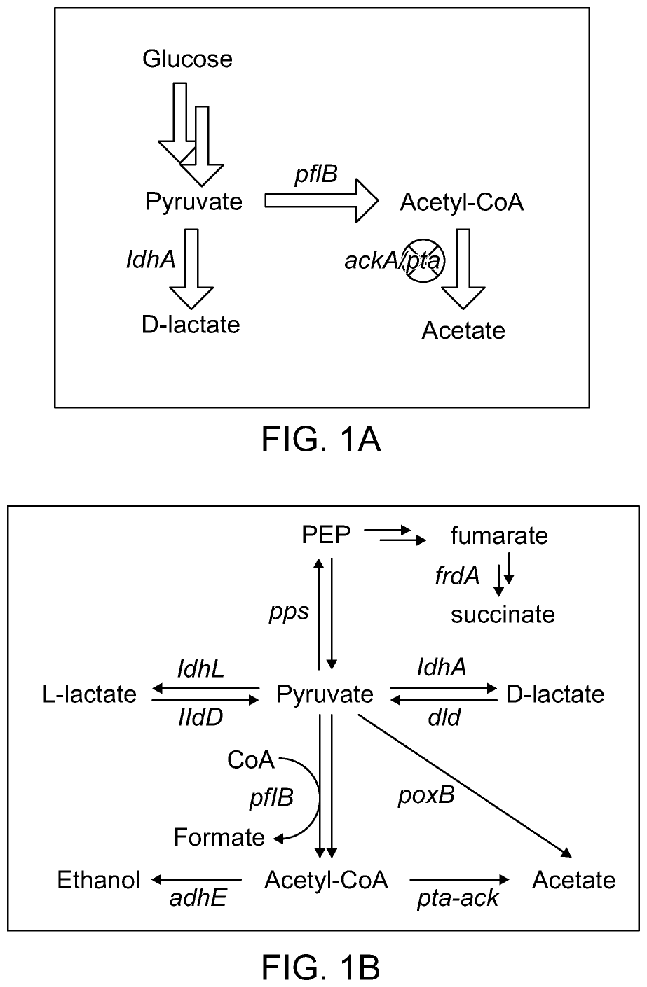

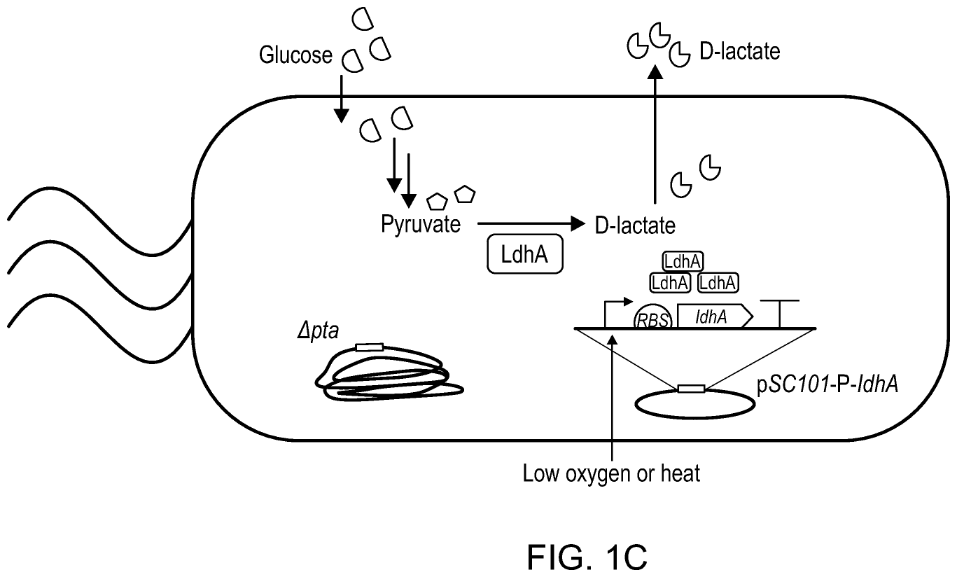

### DETAILED DESCRIPTION

The present disclosure provides recombinant bacteria for production of D-lactate and/or L-lactate, pharmaceutical compositions thereof, and methods of modulating and treating diseases associated with D-lactate and/or L-lactate. The recombinant bacteria are capable of producing D-lactate and/or L-lactate in low-oxygen environments, e.g., the gut. Thus, the recombinant bacteria and pharmaceutical compositions comprising those bacteria are non-pathogenic, and can be used in order to treat and/or prevent conditions associated with autoimmune and inflammatory diseases and disorders.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including, but not limited to”.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with the term “or”, “at least one of” or “one or more of” the elements in a list, unless context clearly indicates otherwise.

The term “about” is used herein to mean within the typical ranges of tolerances in the art, e.g., acceptable variation in time between doses, acceptable variation in dosage unit amount. For example, “about” can be understood as within about 2 standard deviations from the mean. In certain embodiments, about means +10%. In certain embodiments, about means +5%. When about is present before a series of numbers or a range, it is understood that “about” can modify each of the numbers in the series or range.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

As used herein, the term “recombinant bacterial cell” or “recombinant bacterium” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed bacterial cell” or “programmed engineered bacterial cell” is a recombinant, or an engineered bacterial cell, that has been genetically modified from its native state to perform a specific function. In certain embodiments, the programmed or engineered bacterial cell has been modified to express one or more proteins, for example, one or more proteins that have a therapeutic activity or serve a therapeutic purpose. The programmed or engineered bacterial cell may additionally have the ability to stop growing or to destroy itself once the protein(s) of interest have been expressed.

As used herein, a “heterologous” gene or “heterologous sequence” refers to a nucleotide sequence that is not normally found in a given cell or organism in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. For example, a heterologous gene may include a native coding sequence that is a portion of a chimeric gene to include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature.

As used herein, the term “endogenous gene” refers to a native gene in its natural location in the genome of an organism. As used herein, the term “transgene” refers to a gene that has been introduced into the host organism, e.g., host bacterial cell, genome.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, and stem-loop structures. In one embodiment, the regulatory sequence comprises a promoter, e.g., an FNR responsive promoter.

As used herein, a “gene cassette” or “operon” refers to a functioning unit of DNA containing a set of linked genes under the control of a single promoter. The genes are transcribed together into an mRNA strand and then translated for expression. A gene cassette encoding a biosynthetic pathway refers to two or more genes that are required to produce a molecule, e.g., indole-3-acetic acid. In addition to encoding a set of genes capable of producing said molecule, the gene cassette or operon may also comprise additional transcription and translation elements, e.g., a ribosome binding site.

A “D-lactate gene” or “D-lactate biosynthesis gene” are used interchangeably to refer to a gene (or set of genes) capable of producing D-lactate in a biosynthetic pathway. Unmodified bacteria that are capable of producing D-lactate via an endogenous D-lactate biosynthesis pathway include, but are not limited to, Bacillus, Escherichia, Clostridium, Megasphaera, Prevotella, Lactobacillus, Carnobacterium, Lactococcus, Streptococcus, Enterococcus, Vagococcus, Leuconostoc, Oenococcus, Pediococcus, Tetragonococcus, Aerococcus, and Weissella, e.g., Escherichia coli, Bacillus coagulans, Clostridium propionicum, Megasphaera elsdenii, Prevotella ruminicola, Lactobacillus acidophilus, Lactobacillus gasseri, Lactobacillus delbrueckii subsp. bulgaricus, Lactobacillus fermentum, Lactobacillus lactis, Lactobacillus brevis, Lactobacillus helveticus, Lactobacillus plantarum and Lactobacillus reuteri The recombinant bacteria may comprise D-lactate biosynthesis genes from a different species, strain, or substrain of bacteria, or a combination of D-lactate biosynthesis genes from different species, strains, and/or substrains of bacteria. In some embodiments, In some embodiments, the D-lactate gene may comprise ldhA gene. In some embodiments, the ldhA gene is from E. coli. In some embodiments, the gene(s) may be functionally replaced or modified, e.g., codon optimized, for enhanced expression. In other embodiments, one or more ribosome binding sites are added to one or more of the gene(s).

A “L-lactate gene” or “L-lactate biosynthesis gene” are used interchangeably to refer to a gene (or set of genes) capable of producing L-lactate in a biosynthetic pathway. Unmodified bacteria that are capable of producing L-lactate via an endogenous L-lactate biosynthesis pathway include, but are not limited to, Bacillus, Escherichia, Clostridium, Megasphaera, Prevotella, Lactobacillus, Carnobacterium, Lactococcus, Streptococcus, Enterococcus, Vagococcus, Leuconostoc, Oenococcus, Pediococcus, Tetragonococcus, Aerococcus, and Weissella, e.g., Escherichia coli, Bacillus coagulans, Clostridium propionicum, Megasphaera elsdenii, Prevotella ruminicola, Lactobacillus acidophilus, Lactobacillus gasseri, Lactobacillus delbrueckii subsp. bulgaricus, Lactobacillus fermentum, Lactobacillus lactis, Lactobacillus brevis, Lactobacillus helveticus, Lactobacillus plantarum and Lactobacillus reuteri. The recombinant bacteria may comprise L-lactate biosynthesis genes from a different species, strain, or substrain of bacteria, or a combination of L-lactate biosynthesis genes from different species, strains, and/or substrains of bacteria. In some embodiments, In some embodiments, the L-lactate gene may comprise ldhL gene. In some embodiments, the ldhL gene is from Bacillus coagulans. In some embodiments, the gene(s) may be functionally replaced or modified, e.g., codon optimized, for enhanced expression. In other embodiments, one or more ribosome binding sites are added to one or more of the gene(s).

As used herein, the term “ribosome binding site” or “RBS” refers to a sequence of nucleotides upstream of the start codon of an mRNA transcript that is responsible for the recruitment of a ribosome during the initiation of protein translation. In some embodiments, one or more ribosome binding sites are added to one or more of the genes in the gene cassette described herein for enhanced expression. In other embodiments, the sequence for ribosome binding site is optimized for enhanced expression.

As used herein, the term “operably linked” refers a nucleic acid sequence, e.g., a gene or gene cassette for producing a metabolite, that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns. In some embodiments, each gene or gene cassette may be operably linked to a promoter that is induced under low-oxygen conditions.

A “directly inducible promoter” refers to a regulatory region, wherein the regulatory region is operably linked to a gene or a gene cassette encoding a biosynthetic pathway for producing a metabolite, e.g., D-lactate and/or L-lactate. In the presence of an inducer of said regulatory region, a metabolic molecule is expressed.

An “indirectly inducible promoter” refers to a regulatory system comprising two or more regulatory regions, for example, a first regulatory region that is operably linked to a gene encoding a first molecule, e.g., a transcription factor, which is capable of regulating a second regulatory region that is operably linked to a gene or a gene cassette encoding a biosynthetic pathway for producing a metabolite, e.g., D-lactate and/or L-lactate. In the presence of an inducer of the first regulatory region, the second regulatory region may be activated or repressed, thereby activating or repressing production of D-lactate and/or L-lactate. Both a directly inducible promoter and an indirectly inducible promoter are encompassed by “inducible promoter.”

“Exogenous environmental condition(s)” refers to setting(s) or circumstance(s) under which the promoter described above is directly or indirectly induced. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen or anaerobic conditions such as the environment of the mammalian gut. In some embodiments, exogenous environmental conditions are molecules or metabolites that are specific to the mammalian gut, e.g., D-lactate and/or L-lactate. In some embodiments, the gene or gene cassette for producing a therapeutic molecule is operably linked to an oxygen level-dependent promoter. Bacteria have evolved transcription factors that are capable of sensing oxygen levels. Different signaling pathways may be triggered by different oxygen levels and occur with different kinetics.

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression. In some embodiments, the gene or gene cassette for producing a metabolite, e.g., D-lactate and/or L-lactate, is operably linked to an oxygen level-dependent regulatory region such that the metabolite is expressed in low-oxygen, microaerobic, or anaerobic conditions. For example, the oxygen level-dependent regulatory region is operably linked to a D-lactate gene cassette and/or L-lactate gene cassette. In low oxygen conditions, the oxygen level-dependent regulatory region is activated by a corresponding oxygen level-sensing transcription factor, thereby driving expression of the D-lactate gene cassette and/or L-lactate gene cassette.

As used herein, a “non-native” nucleic acid sequence refers to a nucleic acid sequence not normally present in a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the recombinant bacteria comprise a gene cassette that is operably linked to a directly or indirectly inducible promoter that is not associated with said gene cassette in nature, e.g., a FNR-responsive promoter operably linked to a D-lactate gene or gene cassette and/or L-lactate gene or gene cassette.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli e promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ²⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), Pieper (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

“Microorganism” refers to an organism or microbe of microscopic, submicroscopic, or ultramicroscopic size that typically consists of a single cell. Examples of microorganisms include bacteria, viruses, parasites, fungi, certain algae, and protozoa. In some aspects, the microorganism is engineered (“engineered microorganism”) to produce one or more therapeutic molecules. In certain aspects, the microorganism is engineered to import and/or catabolize certain toxic metabolites, substrates, or other compounds from its environment, e.g., the gut. In certain aspects, the microorganism is engineered to synthesize certain beneficial metabolites, molecules, or other compounds (synthetic or naturally occurring) and release them into its environment. In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria are commensal bacteria, which are present in the indigenous microbiota of the gut. Examples of non-pathogenic bacteria include, but are not limited to Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, “stably maintained” or “stable” bacterium is used to refer to a bacterial host cell carrying non-native genetic material, e.g., a D-lactate gene or gene cassette and/or L-lactate gene or gene cassette, which is incorporated into the host genome or propagated on a self-replicating extra-chromosomal plasmid, such that the non-native genetic material is retained, expressed, and/or propagated. The stable bacterium is capable of survival and/or growth in vitro, e.g., in medium, and/or in vivo, e.g., in the gut. For example, the stable bacterium may be a genetically modified bacterium comprising a D-lactate gene and/or L-lactate gene, in which the plasmid or chromosome carrying the D-lactate gene and/or L-lactate gene is stably maintained in the host cell, such that the gene can be expressed in the host cell, and the host cell is capable of survival and/or growth in vitro and/or in vivo.

As used herein, “autoimmune disease or disorder” and “inflammatory disease or disorder” include, but are not limited to, multiple sclerosis, central nervous system inflammation (CNS) inflammation, 2,4,6-trinitrobenzene sulfonic acid (TNBS)-induced colitis, T cell-induced colitis, T cell-induced small bowel inflammation, chronic colitis, rheumatoid arthritis, celiac disease, myasthenia gravis, and B-cell-mediated T-cell-dependent autoimmune disease.

Symptoms associated with the aforementioned diseases and conditions include, but are not limited to, one or more of inflammation, weight gain, obesity, fatigue, hyperlipidemia, hyperphagia, hyperdipsia, polyphagia, polydipsia, polyuria, pain of the extremities, numbness of the extremities, blurry vision, nystagmus, hearing loss, cardiomyopathy, insulin resistance, light sensitivity, pulmonary disease, liver disease, liver cirrhosis, liver failure, kidney disease, kidney failure, seizures, hypogonadism, and infertility.

Autoimmune and inflammatory diseases are associated with a variety of physiological changes, including but not limited to elevated glucose levels, elevated triglyceride levels, elevated cholesterol levels, insulin resistance, high blood pressure, hypogonadism, subfertility, infertility, abdominal obesity, pro-thrombotic conditions, and pro-inflammatory conditions.

As used herein, the term “modulate” and its cognates means to alter, regulate, or adjust positively or negatively a molecular or physiological readout, outcome, or process, to effect a change in said readout, outcome, or process as compared to a normal, average, wild-type, or baseline measurement. Thus, for example, “modulate” or “modulation” includes up-regulation and down-regulation. A non-limiting example of modulating a readout, outcome, or process is effecting a change or alteration in the normal or baseline functioning, activity, expression, or secretion of a biomolecule (e.g., a protein, enzyme, cytokine, growth factor, hormone, metabolite, short chain fatty acid, or other compound). Another non-limiting example of modulating a readout, outcome, or process is effecting a change in the amount or level of a biomolecule of interest, e.g., in the serum and/or the gut lumen. In another non-limiting example, modulating a readout, outcome, or process relates to a phenotypic change or alteration in one or more disease symptoms. Thus, “modulate” is used to refer to an increase, decrease, masking, altering, overriding or restoring the normal functioning, activity, or levels of a readout, outcome or process (e.g., biomolecule of interest, and/or molecular or physiological process, and/or a phenotypic change in one or more disease symptoms).

As used herein, the term “treat” and its cognates refer to an amelioration of a disease or disorder, or at least one discernible symptom thereof. In another embodiment, “treat” refers to an amelioration of at least one measurable physical parameter, not necessarily discernible by the patient. In another embodiment, “treat” refers to inhibiting the progression of a disease or disorder, either physically (e.g., stabilization of a discernible symptom), physiologically (e.g., stabilization of a physical parameter), or both. In another embodiment, “treat” refers to slowing the progression or reversing the progression of a disease or disorder. As used herein, “prevent” and its cognates refer to delaying the onset or reducing the risk of acquiring a given disease or disorder.

Those in need of treatment may include individuals already having a particular medical disorder, as well as those at risk of having, or who may ultimately acquire the disorder. The need for treatment is assessed, for example, by the presence of one or more risk factors associated with the development of a disorder, the presence or progression of a disorder, or likely receptiveness to treatment of a subject having the disorder. Treating diseases may encompass reducing or eliminating associated symptoms, e.g., inflammation, wound healing, and weight gain, and does not necessarily encompass the elimination of the underlying disease or disorder. Treating the diseases described herein may encompass increasing levels of D-lactate and/or L-lactate, or decreasing levels of pyruvate, and does not necessarily encompass the elimination of the underlying disease.

As used herein a “pharmaceutical composition” refers to a preparation of recombinant bacteria with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., disease. A therapeutically effective amount may, for example, be sufficient to treat, prevent, reduce the severity, delay the onset, and/or reduce the risk of occurrence of one or more symptoms of a disease. A therapeutically effective amount, as well as a therapeutically effective frequency of administration, can be determined by methods known in the art and discussed below.

### II. Recombinant Bacteria

The recombinant bacteria disclosed herein comprise a gene or gene cassette for producing a non-native metabolic molecule, e.g., D-lactate and/or L-lactate. In some embodiments, the recombinant bacteria comprise one or more gene(s) or gene cassette(s) which are capable of producing the metabolite, e.g., D-lactate and/or L-lactate.

The recombinant bacteria may express one or more D-lactate biosynthesis genes and/or one or more L-lactate biosynthesis genes (see, e.g., Table 2).

In some embodiments, the recombinant bacterium may comprise a mutation or a deletion in one or more gene(s) selected from formate acetyltransferase 1 (pflB), acetate kinase (ackA), phosphate acetyltransferase (pta), aldehyde dehydrogenase (adhE), methylglyoxyl synthetase (mgsA), fumarase reductase subunit (frdB), fumarase reductase subunit (frdC) and/or phosphofructokinase (pfkA). In some embodiments, the recombinant bacterium may comprise a mutation or deletion in the pflB gene (enconding a formate acetyltransferase 1). In some embodiments, the recombinant bacterium may comprise a mutation or deletion in the ackA gene encoding an acetate kinase. In some embodiments, the recombinant bacterium may comprise a mutation or deletion in the pta gene encoding a phosphate acetyltransferase. In some embodiments, the recombinant bacterium may comprise a mutation or a deletion in the adhE gene encoding an aldehyde dehydrogenase. In some embodiments, the recombinant bacterium may comprise a mutation or deletion in the pfkA gene encoding a phosphofructokinase. In some embodiments, the recombinant bacteria comprises a deletion or mutation is in the mgsA gene encoding a methylglyoxyl synthetase. In some embodiments, the recombinant bacteria comprises a deletion or mutation is in the frdB gene encoding a fumarase reductase subunit. In some embodiments, the recombinant bacteria comprises a deletion or mutation is in the frdC gene encoding a fumarase reductase subunit.

The genes may be codon-optimized, and translational and transcriptional elements may be added. In some embodiments, the gene or gene cassette for producing a metabolic molecule, e.g., D-lactate and/or L-lactate, comprises additional transcription and translation elements, e.g., a ribosome binding site, to enhance expression of the metabolic molecule. One or more ribosome binding sites may be added within a given gene cassette. In some embodiments, a ribosome binding site is added before the ldhA gene and/or ldhL gene. In some embodiments, different ribosome binding sites are added before different genes. In other embodiments, the same ribosome binding site is added before different genes.

Table 1 lists the nucleic acid sequences of exemplary constructs comprising the D-lactate biosynthesis genes, relevant plasmids, exemplary nucleic acid sequences comprising the L-lactate biosynthesis genes, and/or relevant genes to be mutated or deleted. Table 2 lists the polypeptide sequences encoded by the nucleic acid sequences in Table 1.

TABLE 1
Nucleic Acid Sequences

| Construct | Sequence |
|---|---|
| logic 1919 | aaaaatgaagttttaaatcaatctaaagta |
| (SEQ ID | tatatgagtaaacttggtctgacagttacc |
| NO: 1) | aatgcttaatcagtgaggcacctatctcag |
| | cgatctgtctatttcgttcatccatagttg |
| | cctgactccccgtcgtgtagataactacga |
| | tacgggagggcttaccatctggccccagtg |
| | ctgcaatgataccgcgagaaccacgctcac |
| | cggctccagatttatcagcaataaaccagc |
| | cagccggaagggccgagcgcagaagtggtc |
| | ctgcaactttatccgcctccatccagtcta |
| | ttaattgttgccgggaagctagagtaagta |
| | gttcgccagttaatagtttgcgcaacgttg |
| | ttgccattgctacaggcatcgtggtgtcac |
| | gctcgtcgtttggtatggcttcattcagct |
| | ccggttcccaacgatcaaggcgagttacat |
| | gatcccccatgttgtgcaaaaaagcggtta |
| | gctccttcggtcctccgatcgttgtcagaa |
| | gtaagttggccgcagtgttatcactcatgg |
| | ttatggcagcactgcataattctcttactg |
| | tcatgccatccgtaagatgcttttctgtga |
| | ctggtgagtactcaaccaagtcattctgag |
| | aatagtgtatgcggcgaccgagttgctctt |
| | gcccggcgtcaatacgggataataccgcgc |
| | cacatagcagaactttaaaagtgctcatca |
| | ttggaaaacgttcttcggggcgaaaactct |
| | caaggatcttaccgctgttgagatccagtt |
| | cgatgtaacccactcgtgcacccaactgat |
| | cttcagcatcttttactttcaccagcgttt |
| | ctgggtgagcaaaaacaggaaggcaaaatg |
| | ccgcaaaaaagggaataagggcgacacgga |
| | aatgttgaatactcatactcttcctttttc |
| | aatattattgaagcatttatcagggttatt |
| | gtctcatgagcggatacatatttgaatgta |
| | tttagaaaaataaacaaataggggttccgc |
| | gcacatttccccgaaaagtgccacctgacg |
| | tctaagaaaccattattatcatgacattaa |
| | cctataaaaataggcgtatcacgaggccct |
| | ttcgtctcgcgcgtttcggtgatgacggtg |
| | aaaacctctgacacatgcagctcccggaga |
| | cggtcacagcttgtctgtaagcggatgccg |
| | ggagcagacaagcccgtcagggcgcgtcag |
| | cgggtgttggcgggtgtcggggctggctta |
| | actatgcggcatcagagcagattgtactga |
| | gagtgcaccatatgcggtgtgaaataccgc |
| | acagatgcgtaaggagaaaataccgcatca |
| | ggcgccattcgccattcaggctgcgcaact |
| | gttgggaagggcgatcggtgcgggcctctt |
| | cgctattacgccagctggcgaaagggggat |
| | gtgctgcaaggcgattaagttgggtaacgc |
| | cagggttttcccagtcacgacgttgtaaaa |
| | cgacggccagtgcgCTCCCGGAGACGGTCA |
| | CAGCTTGTCaaaaaaaaaccccgcttcggc |
| | ggggtttttttttGGTACCTCATCAGCCAA |
| | ACGTCTCTTCAGGCCACTGACTAGCGATAA |
| | CTTTCCCCACAACGGAACAACTCTCATTGC |
| | ATGGGATCATTGGGTACTGTGGGTTTAGTG |
| | GTTGTAAAAACACCTGACCGCTATCCCTGA |
| | TCAGTTTCTTGAAGGTAAACTCATCACCCC |
| | CAAGTCTGGCTATGCAGAAATCACCTGGCT |
| | CAACAGCCTGCTCAGGGTCAACGAGAATTA |
| | ACATTCCGTCAGGAAAGCTTGGCTTGGAGC |
| | CTGTTGGTGCGGTCATGGAATTACCTTCAA |
| | CCTCAAGCCAGAATGCAGAATCACTGGCTT |
| | TTTTGGTTGTGCTTACCCATCTCTCCGCAT |
| | CACCTTTGGTAAAGGTTCTAAGCTTAGGTG |
| | AGAACATCCCTGCCTGAACATGAGAAAAAA |
| | CAGGGTACTCATACTCACTTCTAAGTGACG |
| | GCTGCATACTAACCGCTTCATACATCTCGT |
| | AGATTTCTCTGGCGATTGAAGGGCTAAATT |
| | CTTCAACGCTAACTTTGAGAATTTTTGTAA |
| | GCAATGCGGCGTTATAAGCATTTAATGCAT |
| | TGATGCCATTAAATAAAGCACCAACGCCTG |
| | ACTGCCCCATCCCCATCTTGTCTGCGACAG |
| | ATTCCTGGGATAAGCCAAGTTCATTTTTCT |
| | TTTTTTCATAAATTGCTTTAAGGCGACGTG |
| | CGTCCTCAAGCTGCTCTTGTGTTAATGGTT |
| | TCTTTTTTGTGCTCATACGTTAAATCTATC |
| | ACCGCAAGGGATAAATATCTAACACCGTGC |
| | GTGTTGACTATTTTACCTCTGGCGGTGATA |
| | ATGGTTGCATaagtgaggatccaaagtgaa |
| | ctctagaaataattttgtttaactttaaga |
| | aggaggtatacatATGAAACTTGCTGTATA |
| | TAGTACCAAACAGTACGACAAAAAGTACCT |
| | TCAACAGGTCAACGAGAGCTTTGGTTTCGA |
| | ACTTGAATTTTTCGACTTTTTACTTACCGA |
| | GAAAACGGCAAAAACGGCGAACGGATGTGA |
| | AGCGGTTTGCATTTTCGTCAACGACGACGG |
| | CAGCCGCCCTGTTTTAGAAGAGTTAAAGAA |
| | ACATGGAGTTAAATACATCGCATTACGTTG |
| | TGCAGGTTTCAACAACGTTGATCTGGATGC |
| | TGCGAAGGAACTGGGATTGAAAGTTGTGCG |
| | CGTGCCCGCTTATGACCCAGAGGCGGTTGC |
| | GGAACACGCTATTGGTATGATGATGACCCT |
| | TAATCGTCGCATCCATCGTGCATATCAGCG |
| | CACGCGCGATGCTAACTTCAGTTTAGAAGG |
| | ATTAACGGGATTTACAATGTACGGGAAGAC |
| | CGCTGGCGTGATTGGCACCGGAAAAATCGG |
| | TGTGGCAATGCTGCGTATCTTGAAGGGGTT |
| | TGGCATGCGTTTGTTAGCATTTGATCCCTA |
| | TCCAAGTGCCGCGGCCCTGGAACTGGGAGT |
| | GGAATATGTTGATTTGCCAACTTTGTTTAG |
| | CGAGTCCGATGTTATCTCATTGCATTGTCC |
| | ACTTACTCCGGAGAATTATCATTTATTGAA |
| | TGAAGCCGCCTTCGAACAAATGAAAAATGG |
| | AGTGATGATCGTAAATACAAGTCGTGGCGC |
| | GTTGATCGATTCGCAGGCAGCGATCGAAGC |
| | GTTAAAAAATCAAAAGATTGGATCACTGGG |
| | CATGGATGTCTATGAAAACGAGCGCGACCT |
| | TTTCTTTGAAGACAAAAGTAATGATGTTAT |
| | CCAAGATGATGTATTTCGCCGTCTGTCGGC |
| | ATGCCATAATGTACTTTTTACGGGTCACCA |
| | AGCATTCCTTACTGCCGAGGCTCTGACTAG |
| | CATTTCACAAACCACTCTTCAGAATCTTTC |
| | AAATCTTGAGAAAGGTGAGACGTGCCCCAA |
| | TGAATTGGTTtaaGCATGCTAATCAGCCGT |
| | GGAATTCGGTCTCaGGAGgtacgcatggca |
| | tggatgaccgatggtagtgtgggctctccc |
| | catgcgagagtagggaactgccaggcatca |
| | aataaaacgaaaggctcagtcgaaagactg |
| | ggcctttcgttttatctgttgtttgtcggt |
| | gaacgctctcctgagtaggacaaatccgcc |
| | gggagcggatttgaacgttgcgaagcaacg |
| | gcccggagggtggcgggcaggacgcccgcc |
| | ataaactgccaggcatcaaattaagcagaa |
| | ggccatcctgacggatggcctttttgcgtg |
| | gccagtgccaagcttgcatgcgtgccagct |
| | gcattaatgaagaaatcatgctggaagaat |
| | aacagctcactcaaaggcggtagtacgggt |
| | tttgctgcccgcaaacgggctgttctggtg |
| | ttgctagtttgttatcagaatcgcagatcc |
| | ggcttcagccggtttgccggctgaaagcgc |
| | tatttcttccagaattgccatgattttttc |
| | cccacgggaggcgtcactggctcccgtgtt |
| | gtcggcagctttgattcgataagcagcatc |
| | gcctgtttcaggctgtctatgtgtgactgt |
| | tgagctgtaacaagttgtctcaggtgttca |
| | atttcatgttctagttgctttgttttactg |
| | gtttcacctgttctattaggtgttacatgc |
| | tgttcatctgttacattgtcgatctgttca |
| | tggtgaacagctttgaatgcaccaaaaact |
| | cgtaaaagctctgatgtatctatctttttt |
| | acaccgttttcatctgtgcatatggacagt |
| | tttccctttgatatgtaacggtgaacagtt |
| | gttctacttttgtttgttagtcttgatgct |
| | tcactgatagatacaagagccataagaacc |
| | tcagatccttccgtatttagccagtatgtt |
| | ctctagtgtggttcgttgtttttgcgtgag |
| | ccatgagaacgaaccattgagatcatactt |
| | actttgcatgtcactcaaaaattttgcctc |
| | aaaactggtgagctgaatttttgcagttaa |
| | agcatcgtgtagtgtttttcttagtccgtt |
| | atgtaggtaggaatctgatgtaatggttgt |
| | tggtattttgtcaccattcatttttatctg |
| | gttgttctcaagttcggttacgagatccat |
| | ttgtctatctagttcaacttggaaaatcaa |
| | cgtatcagtcgggcggcctcgcttatcaac |
| | caccaatttcatattgctgtaagtgtttaa |
| | atctttacttattggtttcaaaacccattg |
| | gttaagccttttaaactcatggtagttatt |
| | ttcaagcattaacatgaacttaaattcatc |
| | aaggctaatctctatatttgccttgtgagt |
| | tttcttttgtgttagttcttttaataacca |
| | ctcataaatcctcatagagtatttgttttc |
| | aaaagacttaacatgttccagattatattt |
| | tatgaatttttttaactggaaaagataagg |
| | caatatctcttcactaaaaactaattctaa |
| | tttttcgcttgagaacttggcatagtttgt |
| | ccactggaaaatctcaaagcctttaaccaa |
| | aggattcctgatttccacagttctcgtcat |
| | cagctctctggttgctttagctaatacacc |
| | ataagcattttccctactgatgttcatcat |
| | ctgagcgtattggttataagtgaacgatac |
| | cgtccgttctttccttgtagggttttcaat |
| | cgtggggttgagtagtgccacacagcataa |
| | aattagcttggtttcatgctccgttaagtc |
| | atagcgactaatcgctagttcatttgcttt |
| | gaaaacaactaattcagacatacatctcaa |
| | ttggtctaggtgattttaatcactatacca |
| | attgagatgggctagtcaatgataattact |
| | agtccttttcctttgagttgtgggtatctg |
| | taaattctgctagacctttgctggaaaact |
| | tgtaaattctgctagaccctctgtaaattc |
| | cgctagacctttgtgtgttttttttgttta |
| | tattcaagtggttataatttatagaataaa |
| | gaaagaataaaaaaagataaaaagaataga |
| | tcccagccctgtgtataactcactacttta |
| | gtcagttccgcagtattacaaaaggatgtc |
| | gcaaacgctgtttgctcctctacaaaacag |
| | accttaaaaccctaaaggcttaagtagcac |
| | cctcgcaagctcgggcaaatcgctgaatat |
| | tccttttgtctccgaccatcaggcacctga |
| | gtcgctgtctttttcgtgacattcagttcg |
| | ctgcgctcacggctctggcagtgaatgggg |
| | gtaaatggcactacaggcgccttttatgga |
| | ttcatgcaaggaaactacccataatacaag |
| | aaaagcccgtcacgggcttctcagggcgtt |
| | ttatggcgggtctgctatgtggtgctatct |
| | gactttttgctgttcagcagttcctgccct |
| | ctgattttccagtctgaccacttcggatta |
| | tcccgtgacaggtcattcagactggctaat |
| | gcacccagtaaggcagcggtatcatcaaca |
| | ggcttacccgtcttactgtcttttctacgg |
| | ggtctgacgctcagtggaacgaaaactcac |
| | gttaagggattttggtcatgagattatcaa |
| | aaaggatcttcacctagatccttttaaatt |
| logic 1920 | caatgcttaatcagtgaggcacctatctca |
| (SEQ ID | gcgatctgtctatttcgttcatccatagtt |
| NO: 2) | gcctgactccccgtcgtgtagataactacg |
| | atacgggagggcttaccatctggccccagt |
| | gctgcaatgataccgcgagaaccacgctca |
| | ccggctccagatttatcagcaataaaccag |
| | ccagccggaagggccgagcgcagaagtggt |
| | cctgcaactttatccgcctccatccagtct |
| | attaattgttgccgggaagctagagtaagt |
| | agttcgccagttaatagtttgcgcaacgtt |
| | gttgccattgctacaggcatcgtggtgtca |
| | cgctcgtcgtttggtatggcttcattcagc |
| | tccggttcccaacgatcaaggcgagttaca |
| | tgatcccccatgttgtgcaaaaaagcggtt |
| | agctccttcggtcctccgatcgttgtcaga |
| | agtaagttggccgcagtgttatcactcatg |
| | gttatggcagcactgcataattctcttact |
| | gtcatgccatccgtaagatgcttttctgtg |
| | actggtgagtactcaaccaagtcattctga |
| | gaatagtgtatgcggcgaccgagttgctct |
| | tgcccggcgtcaatacgggataataccgcg |
| | ccacatagcagaactttaaaagtgctcatc |
| | attggaaaacgttcttcggggcgaaaactc |
| | tcaaggatcttaccgctgttgagatccagt |
| | tcgatgtaacccactcgtgcacccaactga |
| | tcttcagcatcttttactttcaccagcgtt |
| | tctgggtgagcaaaaacaggaaggcaaaat |
| | gccgcaaaaaagggaataagggcgacacgg |
| | aaatgttgaatactcatactcttccttttt |
| | caatattattgaagcatttatcagggttat |
| | tgtctcatgagcggatacatatttgaatgt |
| | atttagaaaaataaacaaataggggttccg |
| | cgcacatttccccgaaaagtgccacctgac |
| | gtctaagaaaccattattatcatgacatta |
| | acctataaaaataggcgtatcacgaggccc |
| | tttcgtctcgcgcgtttcggtgatgacggt |
| | gaaaacctctgacacatgcagctcccggag |
| | 00acggtcacagcttgtctgtaagcggatg |
| | ccgggagcagacaagcccgtcagggcgcgt |
| | cagcgggtgttggcgg60gtgtcggggctg |
| | gcttaactatgcggcatcagagcagattgt |
| | actgagagtgcaccatatgcggtgtgaaat |
| | accgcacagatgcgtaaggagaaaataccg |
| | catcaggcgccattcgccattcaggctgcg |
| | caactgttgggaagggcgatcggtgcgggc |
| | ctcttcgctattacgccagctggcgaaagg |
| | gggatgtgctgcaaggcgattaagttgggt |
| | aacgcca00gggttttcccagtcacgacgt |
| | tgtaaaacgacggccagtgcgCTCCCGGAG |
| | ACGGTCACAGCTTGTCaaaaaaaaaccccg |
| | cttcggcggggtttttttttGGTACCTCAA |
| | GTTGTTCTTATTGGTGGTGTTGCTTTATGG |
| | TTGCATCGTAGTAAATGGTTGTAACAAAAG |
| | CAATTTTTCCGGCTGTCTGTATACAAAAAC |
| | GCCGCAAAGTTTGAGCGAAGTCAATAAACT |
| | CTCTACCCATTCAGGGCAATATCTCTCTTg |
| | gatccaaagtgaactctagaaataattttg |
| | tttaactttaagaaggaggtatacatATGA |
| | AACTTGCTGTATATAGTACCAAACAGTACG |
| | ACAAAAAGTACCTTCAACAGGTCAACGAGA |
| | GCTTTGGTTTCGAACTTGAATTTTTCGACT |
| | TTTTACTTACCGAGAAAACGGCAAAAACGG |
| | CGAACGGATGTGAAGCGGTTTGCATTTTCG |
| | TCAACGACGACGGCAGCCGCCCTGTTTTAG |
| | AAGAGTTAAAGAAACATGGAGTTAAATACA |
| | TCGCATTACGTTGTGCAGGTTTCAACAACG |
| | TTGATCTGGATGCTGCGAAGGAACTGGGAT |
| | TGAAAGTTGTGCGCGTGCCCGCTTATGACC |
| | CAGAGGCGGTTGCGGAACACGCTATTGGTA |
| | TGATGATGACCCTTAATCGTCGCATCCATC |
| | GTGCATATCAGCGCACGCGCGATGCTAACT |
| | TCAGTTTAGAAGGATTAACGGGATTTACAA |
| | TGTACGGGAAGACCGCTGGCGTGATTGGCA |
| | CCGGAAAAATCGGTGTGGCAATGCTGCGTA |
| | TCTTGAAGGGGTTTGGCATGCGTTTGTTAG |
| | CATTTGATCCCTATCCAAGTGCCGCGGCCC |
| | TGGAACTGGGAGTGGAATATGTTGATTTGC |
| | CAACTTTGTTTAGCGAGTCCGATGTTATCT |
| | CATTGCATTGTCCACTTACTCCGGAGAATT |
| | ATCATTTATTGAATGAAGCCGCCTTCGAAC |
| | AAATGAAAAATGGAGTGATGATCGTAAATA |
| | CAAGTCGTGGCGCGTTGATCGATTCGCAGG |
| | CAGCGATCGAAGCGTTAAAAAATCAAAAGA |
| | TTGGATCACTGGGCATGGATGTCTATGAAA |
| | ACGAGCGCGACCTTTTCTTTGAAGACAAAA |
| | GTAATGATGTTATCCAAGATGATGTATTTC |
| | GCCGTCTGTCGGCATGCCATAATGTACTTT |
| | TTACGGGTCACCAAGCATTCCTTACTGCCG |
| | AGGCTCTGACTAGCATTTCACAAACCACTC |
| | TTCAGAATCTTTCAAATCTTGAGAAAGGTG |
| | AGACGTGCCCCAATGAATTGGTTtaaGCAT |
| | GCTAATCAGCCGTGGAATTCGGTCTCaGGA |
| | Ggtacgcatggcatggatgaccgatggtag |
| | tgtgggctctccccatgcgagagtagggaa |
| | ctgccaggcatcaaataaaacgaaaggctc |
| | agtcgaaagactgggcctttcgttttatct |
| | gttgtttgtcggtgaacgctctcctgagta |
| | ggacaaatccgccgggagcggatttgaacg |
| | ttgcgaagcaacggcccggagggtggcggg |
| | caggacgcccgccataaactgccaggcatc |
| | aaattaagcagaaggccatcctgacggatg |
| | gcctttttgcgtggccagtgccaagcttgc |
| | atgcgtgccagctgcattaatgaagaaatc |
| | atgctggaagaataacagctcactcaaagg |
| | cggtagtacgggttttgctgcccgcaaacg |
| | ggctgttctggtgttgctagtttgttatca |
| | gaatcgcagatccggcttcagccggtttgc |
| | cggctgaaagcgctatttcttccagaattg |
| | ccatgattttttccccacgggaggcgtcac |
| | tggctcccgtgttgtcggcagctttgattc |
| | gataagcagcatcgcctgtttcaggctgtc |
| | tatgtgtgactgttgagctgtaacaagttg |
| | tctcaggtgttcaatttcatgttctagttg |
| | ctttgttttactggtttcacctgttctatt |
| | aggtgttacatgctgttcatctgttacatt |
| | gtcgatctgttcatggtgaacagctttgaa |
| | tgcaccaaaaactcgtaaaagctctgatgt |
| | atctatcttttttacaccgttttcatctgt |
| | gcatatggacagttttccctttgatatgta |
| | acggtgaacagttgttctacttttgtttgt |
| | tagtcttgatgcttcactgatagatacaag |
| | agccataagaacctcagatccttccgtatt |
| | tagccagtatgttctctagtgtggttcgtt |
| | gtttttgcgtgagccatgagaacgaaccat |
| | tgagatcatacttactttgcatgtcactca |
| | aaaattttgcctcaaaactggtgagctgaa |
| | tttttgcagttaaagcatcgtgtagtgttt |
| | ttcttagtccgttatgtaggtaggaatctg |
| | atgtaatggttgttggtattttgtcaccat |
| | tcatttttatctggttgttctcaagttcgg |
| | ttacgagatccatttgtctatctagttcaa |
| | cttggaaaatcaacgtatcagtcgggcggc |
| | ctcgcttatcaaccaccaatttcatattgc |
| | tgtaagtgtttaaatctttacttattggtt |
| | tcaaaacccattggttaagccttttaaact |
| | catggtagttattttcaagcattaacatga |
| | acttaaattcatcaaggctaatctctatat |
| | ttgccttgtgagttttcttttgtgttagtt |
| | cttttaataaccactcataaatcctcatag |
| | agtatttgttttcaaaagacttaacatgtt |
| | ccagattatattttatgaatttttttaact |
| | ggaaaagataaggcaatatctcttcactaa |
| | aaactaattctaatttttcgcttgagaact |
| | tggcatagtttgtccactggaaaatctcaa |
| | agcctttaaccaaaggattcctgatttcca |
| | cagttctcgtcatcagctctctggttgctt |
| | tagctaatacaccataagcattttccctac |
| | tgatgttcatcatctgagcgtattggttat |
| | aagtgaacgataccgtccgttctttccttg |
| | tagggttttcaatcgtggggttgagtagtg |
| | ccacacagcataaaattagcttggtttcat |
| | gctccgttaagtcatagcgactaatcgcta |
| | gttcatttgctttgaaaacaactaattcag |
| | acatacatctcaattggtctaggtgatttt |
| | aatcactataccaattgagatgggctagtc |
| | aatgataattactagtccttttcctttgag |
| | ttgtgggtatctgtaaattctgctagacct |
| | ttgctggaaaacttgtaaattctgctagac |
| | cctctgtaaattccgctagacctttgtgtg |
| | ttttttttgtttatattcaagtggttataa |
| | tttatagaataaagaaagaataaaaaaaga |
| | taaaaagaatagatcccagccctgtgtata |
| | actcactactttagtcagttccgcagtatt |
| | acaaaaggatgtcgcaaacgctgtttgctc |
| | ctctacaaaacagaccttaaaaccctaaag |
| | gcttaagtagcaccctcgcaagctcgggca |
| | aatcgct50gaatattccttttgtctccga |
| | ccatcaggcacctgagtcgctgtctttttc |
| | gtgacattcagttcgctgcgctcacggctc |
| | tggcagtgaatgggggtaaatggcactaca |
| | ggcgccttttatggattcatgcaaggaaac |
| | tacccataatacaagaaaagcccgtcacgg |
| | gcttctcagggcgttttatggcgggtctgc |
| | tatgtggtgctatctgactttttgctgttc |
| | agcagttcctgccctctgattttccagtct |
| | gaccacttcggattatcccgtgacaggtca |
| | ttcagactggctaatgcacccagtaaggca |
| | gcggtatcatcaacaggcttacccgtctta |
| | ctgtcttttctacggggtctgacgctcagt |
| | ggaacgaaaactcacgttaagggattttgg |
| | tcatgagattatcaaaaaggatcttcacct |
| | agatccttttaaattaaaaatgaagtttta |
| | aatcaatctaaagtatatatgagtaaactt |
| | ggtctgacagttac |
| ldhA | ATGAAACTTGCTGTATATAGTACCAAACAG |
| (SEQ ID | TACGACAAAAAGTACCTTCAACAGGTCAAC |
| NO: 3) | GAGAGCTTTGGTTTCGAACTTGAATTTTTC |
| | GACTTTTTACTTACCGAGAAAACGGCAAAA |
| | ACGGCGAACGGATGTGAAGCGGTTTGCATT |
| | TTCGTCAACGACGACGGCAGCCGCCCTGTT |
| | TTAGAAGAGTTAAAGAAACATGGAGTTAAA |
| | TACATCGCATTACGTTGTGCAGGTTTCAAC |
| | AACGTTGATCTGGATGCTGCGAAGGAACTG |
| | GGATTGAAAGTTGTGCGCGTGCCCGCTTAT |
| | GACCCAGAGGCGGTTGCGGAACACGCTATT |
| | GGTATGATGATGACCCTTAATCGTCGCATC |
| | CATCGTGCATATCAGCGCACGCGCGATGCT |
| | AACTTCAGTTTAGAAGGATTAACGGGATTT |
| | ACAATGTACGGGAAGACCGCTGGCGTGATT |
| | GGCACCGGAAAAATCGGTGTGGCAATGCTG |
| | CGTATCTTGAAGGGGTTTGGCATGCGTTTG |
| | TTAGCATTTGATCCCTATCCAAGTGCCGCG |
| | GCCCTGGAACTGGGAGTGGAATATGTTGAT |
| | TTGCCAACTTTGTTTAGCGAGTCCGATGTT |
| | ATCTCATTGCATTGTCCACTTACTCCGGAG |
| | AATTATCATTTATTGAATGAAGCCGCCTTC |
| | GAACAAATGAAAAATGGAGTGATGATCGTA |
| | AATACAAGTCGTGGCGCGTTGATCGATTCG |
| | CAGGCAGCGATCGAAGCGTTAAAAAATCAA |
| | AAGATTGGATCACTGGGCATGGATGTCTAT |
| | GAAAACGAGCGCGACCTTTTCTTTGAAGAC |
| | AAAAGTAATGATGTTATCCAAGATGATGTA |
| | TTTCGCCGTCTGTCGGCATGCCATAATGTA |
| | CTTTTTACGGGTCACCAAGCATTCCTTACT |
| | GCCGAGGCTCTGACTAGCATTTCACAAACC |
| | ACTCTTCAGAATCTTTCAAATCTTGAGAAA |
| | GGTGAGACGTGCCCCAATGAATTGGTT |
| ldhL | ATGAAAAAGGTCAATCGTATTGCAGTGGTT |
| (SEQ ID | GGAACGGGTGCAGTTGGTACAAGTTACTGC |
| NO: 4) | TACGCCATGATTAATCAGGGTGTTGCAGAA |
| | GAGCTTGTTTTAATCGATATTAACGAAGCA |
| | AAAGCAGAAGGGGAAGCCATGGACCTGAAC |
| | CACGGCCTGCCATTTGCGCCTACGCCGACC |
| | CGCGTTTGGAAAGGCGATTATTCCGATTGC |
| | GGCACTGCCGATCTTGTTGTCATTACGGCA |
| | GGTTCCCCGCAAAAACCGGGCGAAACAAGG |
| | CTTGATCTTGTTTCCAAAAACGCAAAAATT |
| | TTTAAAGGCATGATTAAGAGCATCATGGAC |
| | AGCGGCTTTAACGGGATTTTTCTTGTTGCC |
| | AGCAACCCGGTTGACATTTTGACATATGTA |
| | ACTTGGAAAGAGTCCGGCCTGCCGAAAGAA |
| | CATGTTATCGGTTCGGGCACAGTGCTTGAC |
| | TCCGCGCGTCTCCGCAACTCTTTGAGCGCC |
| | CAATTTGGAATTGACCCGCGCAATGTGCAT |
| | GCTGCGATTATCGGCGAACACGGCGATACG |
| | GAACTTCCGGTATGGAGCCATACAAATATC |
| | GGTTACGATACGATTGAAAGCTATCTACAA |
| | AAAGGAATTATTGACGAAAAGACGTTAGAT |
| | GACATTTTTGTCAATACGAGAGATGCGGCT |
| | TATCATATTATTGAACGAAAAGGGGCCACA |
| | TTTTACGGCATCGGGATGTCCCTGACCCGG |
| | ATTACAAGGGCAATCCTGAACAATGAAAAC |
| | AGCGTATTGACGGTCTCTGCATTTCTTGAA |
| | GGCCAATACGGAAACAGCGATGTGTACGTT |
| | GGCGTTCCGGCCATCATCAATCGCCAGGGC |
| | ATCCGTGAAGTGGTTGAAATCAAACTGAAC |
| | GAAAAAGAACAGGAACAGTTCAATCATTCT |
| | GTAAAAGTGCTAAAAGAAACGATGGCACCT |
| | GTATTGT |
| pta nucleic acid | atgctgatccctaccggaaccagcgtcggt |
| sequence | ctgaccagcgtcagccttggcgtgatccgt |
| (SEQ ID | gcaatggaacgcaaaggcgttcgtctgagc |
| NO: 5) | gttttcaaacctatcgctcagccgcgtacc |
| | ggtggcgatgcgcccgatcagactacgact |
| | atcgtgcgtgcgaactcttccaccacgacg |
| | gccgctgaaccgctgaaaatgagctacgtt |
| | gaaggtctgctttccagcaatcagaaagat |
| | gtgctgatggaagagatcatcgcgaactac |
| | cacgctaacaccaaagacgctgaagtcgtt |
| | ctggtggaaggtctggtcccgacacgtaag |
| | caccagtttgcccagtctctgaactacgaa |
| | atcgccaaaacgctgaacgcagaaatcgtc |
| | ttcgttatgtctcagggcactgatactccg |
| | gaacagttgaaagagcgtatcgaactgact |
| | cgcaacagcttcggcggtgcaaaaaacacc |
| | aatattaccggcgttatcgttaacaaactg |
| | aacgctccggttgatgagcagggtcgtacc |
| | cgtccggatctgtccgagatttttgacgac |
| | tccaccaaagcaaaagtgaacaacgttgat |
| | ccggcgaagctgcaagaa50tccagcccgc |
| | tgccggttctcggcgctgtgccgtggagct |
| | ttgacctgatcgcgactcgtgcgatcgata |
| | tggctcgccacctgaatgcgaccatcatca |
| | acgaaggcgacatcaatactcgccgcgtta |
| | aatccgtcactttctgcgcacgcagcattc |
| | cgcacatgctggagcacttccgtgccggtt |
| | ctctgctggtgacttccgcagaccgccctg |
| | acgtgctggttgccgcttgcctggctgcca |
| | tgaacggcgtagaaatcggtgccctgctgc |
| | tgactggcggctacgaaatggacgcgcgca |
| | tttctaaactgtgcgaacgtgctttcgcta |
| | ctggcctgccggtatttatggtgaacacca |
| | acacctggcagacttctcttagcctgcaga |
| | gcttcaacctggaagttccggttgacgatc |
| | atgagcgtatcgaaaaagttcaggaatacg |
| | tggctaactacatcaacgctgactggatcg |
| | attctctgactgccacttctgagcgcagcc |
| | gtcgtctgtctccgccagcgttccgttatc |
| | agctgactgaacttgcgcgcaaagcgggca |
| | aacgtatcgttctgccggaaggtgacgaac |
| | cgcgtaccgttaaagcagccgctatctgtg |
| | ctgaacgtggtatcgcaacttgcgtactgc |
| | tgggtaatccggcagagatcaaccgtgttg |
| | cagcctctcagggtgtagaactgggtgcag |
| | gcattgaaatcgttgatccagaagtggttc |
| | gcgaaaactatgttggtcgtctggtcgaac |
| | tgcgtaagaacaaaggcatgaccgaaaccg |
| | ttgcccgcgaacagctggaagacaacgtgg |
| | ttctcggtacgctgatgctggaacaagatg |
| | aagttgatggtctggtttccggtgctgttc |
| | acaccaccgcaaacaccatccgtccgccgc |
| | tgcagctgatcaaaactgcaccgggcagct |
| | ccctggtatcttccgtgttcttcatgctgt |
| | tgccggaacaggtttacgtttacggtgact |
| | gtgcgatcaacccggatccgaccgcagaac |
| | agctggcagaaatcgcgattcagtccgctg |
| | attccgctgcggccttcggtatcgaaccgc |
| | gcgttgctatgctctcctactccaccggta |
| | cttctggtgctggtagcgacgtagaaaaag |
| | ttcgcgaagcaactcgtctggcgcaggaaa |
| | aacgtcctgatctgatgatcgacggtccgc |
| | tgcagtacgacgctgcggtaatggctgacg |
| | ttgcgaaatccaaagcaccgaactctccgg |
| | ttgcaggtcgcgctaccgtgttcatcttcc |
| | cggatctgaacaccggtaacaccacctaca |
| | aagcggtacagcgttctgctgacctgatct |
| | ctatcggaccgatgctgcagggtatgcgca |
| | agccggttaacgacctgtcccgtggcgcac |
| | tggttgatgatatcgtctacaccatcgcgc |
| | tgactgcgattcagtctgcacagcagcagt |
| | aa |
| pflB nucleic acid | atgtccgagcttaatgaaaagttagccaca |
| sequence | gcctgggaaggttttaccaaaggtgactgg |
| (SEQ ID | cagaatgaagtaaacgtccgtgacttcatt |
| NO: 6) | cagaaaaactacactccgtacgagggtgac |
| | gagtccttcctggctggcgctactgaagcg |
| | accaccaccctgtgggacaaagtaatggaa |
| | ggcgttaaactggaaaaccgcactcacgcg |
| | ccagttgactttgacaccgctgttgcttcc |
| | accatcacctctcacgacgctggttacatc |
| | aacaagcagcttgagaaaatcgttggtctg |
| | cagactgaagctccgctgaaacgtgctctt |
| | atcccgttcggtggtatcaaaatgatcgaa |
| | ggttcctgcaaagcgtacaaccgcgaactg |
| | gacccgatgatcaaaaaaatcttcactgaa |
| | taccgtaaaactcacaaccagggcgtgttc |
| | gacgtttacactccggacatcctgcgttgc |
| | cgtaaatccggtgttctgaccggtctgcca |
| | gatgcttatggccgtggtcgtatcatcggt |
| | gactaccgtcgcgttgcgctgtacggtatc |
| | gactacctgatgaaagacaaacttgcacag |
| | ttcacctctctgcaggctgatctggaaaac |
| | ggcgtaaacctggaacagactatccgtctg |
| | cgcgaagaaatcgctgaacagcaccgcgct |
| | ctgggtcagatgaaagaaatggctgcgaaa |
| | tacggctacgacatctctggtccggctacc |
| | aacgctcaggaagctatccagtggacttac |
| | ttcggctacctggctgctgttaagtctcag |
| | aacggtgctgcaatgtccttcggtcgtacc |
| | tccaccttcctggatgtgtacatcgaacgt |
| | gacctgaaagctggcaagatcaccgaacaa |
| | gaagcgcaggaaatggttgaccacctggtc |
| | atgaaactgcgtatggttcgcttcctgcgt |
| | actccggaatacgatgaactgttctctggc |
| | gacccgatctgggcaaccgaatctatcggt |
| | ggtatgggcctcgacggtcgtaccctggtt |
| | accaaaaacagcttccgtttcctgaacacc |
| | ctgtacaccatgggtccgtctccggaaccg |
| | aacatgaccattctgtggtctgaaaaactg |
| | ccgctgaacttcaagaaattcgccgctaaa |
| | gtgtccatcgacacctcttctctgcagtat |
| | gagaacgatgacctgatgcgtccggacttc |
| | aacaacgatgactacgctattgcttgctgc |
| | gtaagcccgatgatcgttggtaaacaaatg |
| | cagttcttcggtgcgcgtgcaaacctggcg |
| | aaaaccatgctgtacgcaatcaacggcggc |
| | gttgacgaaaaactgaaaatgcaggttggt |
| | ccgaagtctgaaccgatcaaaggcgatgtc |
| | ctgaactatgatgaagtgatggagcgcatg |
| | gatcacttcatggactggctggctaaacag |
| | tacatcactgcactgaacatcatccactac |
| | atgcacgacaagtacagctacgaagcctct |
| | ctgatggcgctgcacgaccgtgacgttatc |
| | cgcaccatggcgtgtggtatcgctggtctg |
| | tccgttgctgctgactccctgtctgcaatc |
| | aaatatgcgaaagttaaaccgattcgtgac |
| | gaagacggtctggctatcgacttcgaaatc |
| | gaaggcgaatacccgcagtttggtaacaac |
| | gatccgcgtgtagatgacctggctgttgac |
| | ctggtagaacgtttcatgaagaaaattcag |
| | aaactgcacacctaccgtgacgctatcccg |
| | actcagtctgttctgaccatcacttctaac |
| | gttgtgtatggtaagaaaactggtaacacc |
| | ccagacggtcgtcgtgctggcgcgccgttc |
| | ggaccgggtgctaacccgatgcacggtcgt |
| | gaccagaaaggtgctgtagcgtctctgact |
| | tccgttgctaaactgccgtttgcttacgct |
| | aaagatggtatctcctacaccttctctatc |
| | gttccgaacgcactgggtaaagacgacgaa |
| | gttcgtaagaccaacctggctggtctgatg |
| | gatggttacttccaccacgaagcatccatc |
| | gaaggtggtcagcacctgaacgttaacgtg |
| | atgaaccgtgaaatgctgctcgacgcgatg |
| | gaaaacccggaaaaatatccgcagctgacc |
| | atccgtgtatctggctacgcagtacgtttc |
| | aactcgctgactaaagaacagcagcaggac |
| | gttattactcgtaccttcactcaatctatg |
| | taa |
| ackA nucleic acid | atgtcgagtaagttagtactggttctgaac |
| sequence | tgcggtagttcttcactgaaatttgccatc |
| (SEQ ID | atcgatgcagtaaatggtgaagagtacctt |
| NO: 7) | tctggtttagccgaatgtttccacctgccc |
| | gaagcacgtatcaaatggaaaatggacggc |
| | aataaacaggaagcggctttaggtgcaggc |
| | gccgctcacagcgaagcgctcaactttatc |
| | gttaatactattctggcacaaaaaccagaa |
| | ctgtctgcgcagctgactgctatcggtcac |
| | cgtatcgtacacggcggcgaaaagtatacc |
| | agctccgtagtgatcgatgagtctgttatt |
| | cagggtatcaaagatgcagcttcttttgca |
| | ccgctgcacaacccggctcacctgatcggt |
| | atcgaagaagctctgaaatctttcccacag |
| | ctgaaagacaaaaacgttgctgtatttgac |
| | accgcgttccaccagactatgccggaagag |
| | tcttacctctacgccctgccgtacaacctg |
| | tacaaagagcacggcatccgtcgttacggc |
| | gcgcacggcaccagccacttctatgtaacc |
| | caggaagcggcaaaaatgctgaacaaaccg |
| | gtagaagaactgaacatcatcacctgccac |
| | ctgggcaacggtggttccgtttctgctatc |
| | cgcaacggtaaatgcgttgacacctctatg |
| | ggcctgaccccgctggaaggtctggtcatg |
| | ggtacccgttctggtgatatcgatccggcg |
| | atcatcttccacctgcacgacaccctgggc |
| | atgagcgttgacgcaatcaacaaactgcta |
| | accaaagagtctggcctgctgggtctgacc |
| | gaagtgaccagcgactgccgctatgttgaa |
| | gacaactacgcgacgaaagaagacgcgaag |
| | cgcgcaatggacgtttactgccaccgcctg |
| | gccaaatacatcggtgcctacactgcgctg |
| | atggatggtcgtctggacgctgttgtattc |
| | accggtggtatcggtgaaaatgccgcgatg |
| | gttcgtgaactgtctctgggcaaactgggc |
| | gtgctgggctttgaagttgatcatgaacgc |
| | aacctggctgcacgtttcggcaaatctggt |
| | ttcatcaacaaagaaggtacccgtcctgcg |
| | gtggttatcccaaccaacgaagaactggtt |
| | atcgcgcaagacgcgagccgcctgactgcc |
| | tga |
| adhE nucleic acid | atggctgttactaatgtcgctgaacttaac |
| sequence (SEQ ID | gcactcgtagagcgtgtaaaaaaagcccag |
| NO: 8) | cgtgaatatgccagtttcactcaagagcaa |
| | gtagacaaaatcttccgcgccgccgctctg |
| | gctgctgcagatgctcgaatcccactcgcg |
| | aaaatggccgttgccgaatccggcatgggt |
| | atcgtcgaagataaagtgatcaaaaaccac |
| | tttgcttctgaatatatctacaacgcttat |
| | aaagatgaaaaaacctgtggtgttctgtct |
| | gaagacgacacttttggtaccatcactatc |
| | gctgaacccatcggtattatttgcggtatc |
| | gttccgaccactaacccgacttcaactgct |
| | atcttcaaatcgctgatcagcctgaagacc |
| | cgtaacgccattatcttctccccgcacccg |
| | cgtgcaaaagatgcaaccaacaaagcggct |
| | gatatcgttctacaggctgctatcgctgcc |
| | ggtgctccgaaagatctgatcggctggatc |
| | gatcaaccttctgttgagctgtctaacgca |
| | ctgatgcaccacccagacatcaacctgatc |
| | ctcgcgactggtggtccgggcatggttaaa |
| | gccgcatacagctccggtaaaccagctatc |
| | ggcgtaggcgcgggcaacactccggttgtt |
| | atcgatgaaactgctgatatcaaacgtgca |
| | gttgcatctgtactgatgtccaaaaccttc |
| | gacaacggtgtaatctgtgcttctgaacag |
| | tctgttgttgttgttgactctgtttatgac |
| | gcagtacgtgaacgtttcgcaacccacggc |
| | ggctatctgttgcagggtaaagagctgaaa |
| | gctgttcaggacgttatcctgaaaaacggt |
| | gcgctgaacgcggctatcgttggtcagcca |
| | gcctataaaattgctgaactggcaggcttc |
| | tctgtaccagaaaacaccaagattctgatc |
| | ggtgaagtgaccgttgttgatgaaagcgaa |
| | ccgttcgcacatgaaaaactgtccccgact |
| | ctggcaatgtaccgtgctaaagatttcgaa |
| | gacgcggtagaaaaagcagagaaactggtt |
| | gctatgggcggtatcggtcatacctcttgc |
| | ctgtacactgaccaggataaccaaccggct |
| | cgcgtttcttacttcggtcagaaaatgaaa |
| | acggctcgtatcctgattaacaccccggct |
| | tctcagggtggtatcggtgacctgtataac |
| | ttcaaactcgcaccttccctgactctgggt |
| | tgtggttcctggggtggtaactccatctct |
| | gaaaacgttggtccgaaacacctgatcaac |
| | aagaaaaccgttgctaagcgagctgaaaac |
| | atgttgtggcacaaacttccgaaatctatc |
| | tacttccgccgtggctccctgccaatcgcg |
| | ctggatgaagtgattactgatggccacaaa |
| | cgtgcgctcatcgtgactgaccgcttcctg |
| | ttcaacaatggttatgctgatcagatcact |
| | tccgtattgaaagcagcaggcgttgaaact |
| | gaagtcttcttcgaagtagaagctgacccg |
| | accctgagcatcgttcgtaaaggtgcagaa |
| | ctggcaaactccttcaaaccagacgtgatt |
| | atcgcgctgggtggaggttccccgatggac |
| | gctgcgaagatcatgtgggttatgtacgaa |
| | catccggaaactcacttcgaagaactggcg |
| | ctgcgctttatggatatccgtaaacgtatc |
| | tacaagttcccgaaaatgggtgtgaaagcg |
| | aaaatgatcgctgtcaccaccacttctggt |
| | acaggttctgaagtcactccgtttgcggtt |
| | gtaactgacgacactactggtcagaaatat |
| | ccgctggcagactatgcactgaccccggat |
| | atggcgattgtcgacgccaacctggttatg |
| | gacatgccgaagtccctgtgtgctttcggt |
| | ggtctggacgcagtaactcacgccatggaa |
| | gcttatgtttctgtactggcatctgagttc |
| | tctgatggtcaggctctgcaggcactgaaa |
| | ctgctgaaagaatatctgccagcgtcctac |
| | cacgaagggtctaaaaatccggtagcgcgt |
| | gaacgtgttcacagtgcagcgactatcgcg |
| | ggtatcgcgtttgcgaacgccttcctgggt |
| | gtatgtcactcaatggcgcacaaactgggt |
| | tcccagttccatattccgcacggtctggca |
| | aacgccctgctgatttgtaacgttattcgc |
| | tacaacgcgaatgacaacccgaccaagcag |
| | actgcattcagccagtatgaccgtccgcag |
| | gctcgccgtcgttatgctgaaattgctgac |
| | cacctgggtctgagcgtccgaaatctatcc |
| | gtgaagctggcgttcaggaagcagacttcc |
| | tggcgaacgtggataaactgtctgaagatg |
| | cattcgatgaccagtgcaccggcgctaacc |
| | cgcgttacccgctgatctccgagctgaaac |
| | agattctgctggatacctactacggtcgtg |
| | attatgtagaaggcgaaactgcagcgaaga |
| | aagaagctgctccggctaaagctgagaaaa |
| | aagcgaaaaaatccgcttaa |
| pfkA nucleic acid | atgtgcaagaagacttccggcaacagattt |
| sequence (SEQ ID | cattttgcattccaaagttcagaggtagtc |
| NO: 9) | atgattaagaaaatcggtgtgttgacaagc |
| | ggcggtgatgcgccaggcatgaacgccgca |
| | attcgcggggttgttcgttctgcgctgaca |
| | gaaggtctggaagtaatgggcatttatgac |
| | ggctatctgggtctgtatgaagaccgtatg |
| | gtacagctagaccgttacagcgtttctgac |
| | atgatcaaccgtggtggtacgttcctcggt |
| | tctgcgcgtttcccggaattccgcgacgag |
| | aacatccgcgccgtggctatcgaaaacctg |
| | aaaaaacgtgggatcgacgcgctggtggtt |
| | atcggcggtgacggttcctacatgggtgca |
| | atgcgtctgaccgaaatgggcttcccgtgc |
| | atcggcctgccgggcactatcgacaacgac |
| | atcaaaggcactgactacactatcggtttc |
| | ttcactgcgctgagcaccgttgtagaagcg |
| | atcgaccgtctgcgtgacacctcttcttct |
| | caccagcgtatttccgtggtggaagtgatg |
| | ggccgttattgtggcgatctgacgttggct |
| | gcggctattgccggcggctgtgaattcgtt |
| | gtggttccggaagttgaattcagccgtgaa |
| | gacctggtaaacgaaatcaaagcgggtatc |
| | gcgaaaggtaaaaaacacgcgatcgtggcg |
| | attaccgaacatatgtgtgatgttgacgaa |
| | ctggcgcatttcatcgagaaagaaaccggt |
| | cgtgaaacccgcgcaactgtgctgggccac |
| | atccagcgcggtggttctccggtgccttac |
| | gaccgtattctggcttcccgtatgggcgct |
| | tacgctatcgagctgctgctggcaggttac |
| | ggtggtcgttgcgtaggtatccagaacgaa |
| | cagctaaactgtattaa |
| frdA nucleic acid | gtgcaaacctttcaagccgatcttgccatt |
| sequence (SEQ ID | gtaggcgccggtggcgcgggattacgtgct |
| NO: 10) | gcaattgctgccgcgcaggcaaatccaaat |
| | gcaaaaatcgcactaatctcaaaagtatac |
| | ccgatgcgtagccataccgttgctgcagaa |
| | gggggtgagcaggatgtcgtggattatttc |
| | gtccaccactgcccaaccgaaatgacccaa |
| | ctggaactgtgggggtgcccatggagccgt |
| | cgcccggatggtagcgtcaacgtacgtcgc |
| | ttcggcggcatgaaaatcgagcgcacctgg |
| | ttcgccgccgataagaccggcttccatatg |
| | ctgcacacgctgttccagacctctctgcaa |
| | ttcccgcagatccagcgttttgacgaacat |
| | ttcgtgctggatattctggttgatgatggt |
| | catgttcgcggcctggtagcaatgaacatg |
| | atggaaggcacgctggtgcagatccgtgct |
| | aacgcggtcgttatggctaccggcggtgcg |
| | ggtcgcgtttatcgttacaacaccaacggc |
| | ggcatcgttaccggtgacggtatgggtatg |
| | gcgctaagccacggcgttccgctgcgtgac |
| | atggaattcgttcagtatcacccaaccggt |
| | ctaccaggttccggtatcctgatgaccgaa |
| | ggctgccgcggtgaaggtggtattctggtc |
| | aacaaaaatggctaccgttatctgcaagat |
| | tacggcatgggcccggaaactccgctgggc |
| | gagccgaaaaacaaatatatggaactgggt |
| | ccacgcgacaaagtttctcaggccttctgg |
| | cacgaatggcgtaaaggcaacaccatctcc |
| | acgccacgtggcgatgtggtttacctcgac |
| | ctgcgtcacctcggcgagaaaaaactgcat |
| | gaacgtctgccgttcatctgcgaactggcg |
| | aaagcgtacgttggcgtcgatccggttaaa |
| | gaaccgattccggtacgtccgaccgcacac |
| | tacaccatgggcggtatcgaaaccgatcag |
| | aactgtgaaacccgcattaaaggtctgttc |
| | gccgtgggtgaatgttcctctgttggtctg |
| | cacggtgcgaaccgtctgggctccaactcg |
| | ctggcggaactggtggtcttcggtcgtctg |
| | gccggtgaacaagcgacagagcgtgcagca |
| | actgccggtaatggcaacgaagcggcaatt |
| | gaagcgcaggcagctggcgttgaacaacgt |
| | ctgaaagatctggttaaccaggatggcggc |
| | gaaaactgggctaagatccgcgacgaaatg |
| | ggcatggcaatggaagaaggttgcggtatc |
| | taccgtacgccggaactgatgcagaaaacc |
| | atcgacaagctggcagagctgcaggaacgc |
| | ttcaagcgcgtgcgcatcaccgacacttcc |
| | agcgtgttcaacaccgacctgctctacacc |
| | attgagctgggccacggtctga00acgttg |
| | ctgaatgtatggcgcactccgcaatggcac |
| | gtaaagagtcccgcggcgcgcaccagcgtc |
| | tggacgaaggttgcaccgagcgtgacgacg |
| | tcaacttcctcaaacacaccctcgccttcc |
| | gcgatgctgatggcacgactcgcctggagt |
| | acagcgacgtgaagattactacgctgccgc |
| | cagctaaacgtgtttacggtggcgaagcgg |
| | atgcagccgataaggcggaagcagccaata |
| | agaaggagaaggcgaatggctga |
| poxB nucleic acid | gcgactctctgaacggtcttagtgacagtc |
| sequence (SEQ ID | ttaatcgcatgggcaccatcgagtggatgt |
| NO: 11) | ccacccggcatgaagaagtggcggcgtttg |
| | ccgctggcgctgaagcacaacttagcggag |
| | aactggcggtctgtgccggatcgtgcggtc |
| | ccggcaacctgcacttaatcaacggcctgt |
| | tcgattgccaccgcaatcacgttccggtac |
| | tggcgattgccgctcatattccctccagcg |
| | aaattggcagcggctatttccaggaaaccc |
| | acccacaagagctattccgcgaatgtagtc |
| | actattgcgagcttgtttccagcccagagc |
| | agatcccacaagtgctggcaattgctatgc |
| | gcaaagcggtgcttaaccgtggcgtttccg |
| | ttgttgtgttaccgggcgacgtggcgttaa |
| | aacctgcgccagaaggggcaactacccact |
| | ggtatcatgcgccacagccggtagtaacac |
| | cggaagaagaagagttacgcaaactggcgc |
| | aactgctgcgttattccagcaatatcgccc |
| | tgatgtgtggcagcggctgtgcgggggcgc |
| | ataaagagttagttgagtttgccgggaaaa |
| | ttaaagcgcctatagttcatgccctgcgcg |
| | gtaaagagcatgtcgaatacgataatccgt |
| | atgatgtcggaatgacgggattaatcggct |
| | tctcgtcaggtttccataccatgatgaatg |
| | ccgatacgttagtgctgctcggcacgcaat |
| | ttccctaccgcgcgttctacccgaccgatg |
| | ccaaaattattcagattgatatcaacccag |
| | ccagcatcggcgcgcatagcaaggtagata |
| | tggcgctggtcggcgatatcaaatcaaccc |
| | tgcgggcattgctgccactggtggaagaaa |
| | aaaccgatcgcaagtttctggataaagcgc |
| | tggaagattaccgcgacgcccgcaaagggc |
| | tggatgatttagctaaaccgagcgagaaag |
| | ccattcacccgcaatatctggcgcagcaaa |
| | ttagtcattttgccgccgatgacgccatct |
| | ttacctgtgacgttggtacgccaacggtgt |
| | gggcggcacgttatctgaaaatgaacggca |
| | agcgtcgtctgttaggttcgtttaaccacg |
| | gttcgatggctaacgccatgccgcaggcgc |
| | tgggtgcgcaggcaaccgagccggaacgtc |
| | aggtggtcgccatgtgcggcgatggcggtt |
| | tcagtatgttgatgggcgatttcctctcag |
| | taatgcagatgaaattgccagtgaaaatta |
| | tcgtctttaataacagcgtgctgggctttg |
| | tggcgatggagatgaaagccggaggctacc |
| | tgacagacggtactgagctgcacgacacca |
| | actttgcccgaattgccgaagcctgcggca |
| | ttacgggtattcgtgtagaaaaagcgtctg |
| | aaatcgatgaagctctgcaacgcgccttct |
| | ccatcgacggtccggtgttggtggatgtgg |
| | tggtcgccaaagaagaattagccattccac |
| | cgcagatcaaacttgaacaggccaaaggtt |
| | tcagcctgtatatgctgcgcgcaatcatca |
| | gcgggcgcggtgatgaagtgatcgaactgg |
| | cgaaaacgaactggctaaggtaa |
| pps nucleic acid | atgtccaacaatggctcgtcaccgctggtg |
| sequence (SEQ ID | ctttggtataaccaactcggcatgaatgat |
| NO: 12) | gtagacagggttgggggcaaaaatgcctcc |
| | ctgggtgaaatgattactaacctttccgga |
| | atgggtgtttccgttccgaatggtttcgcc |
| | acaaccgccgacgcgtttaaccagtttctg |
| | gaccaaagcggcgtaaaccagcgcatttat |
| | gaactgctggataaaacggatattgacgat |
| | gttacccagcttgcgaaagcgggcgcgcaa |
| | atccgccagtggattatcgacactcccttc |
| | cagcctgagctggaaaacgccatccgcgaa |
| | gcctatgcacagctttccgccgatgacgaa |
| | aacgcttcgtttgcggtgcgttcctccgct |
| | actgcagaagatatgccggacgcttctttt |
| | gccggtcagcaggaaactttcctcaacgtt |
| | cagggttttgacgccgttctcgtggcagtg |
| | aagcatgtatttgcttctctgtttaacgat |
| | cgcgccatctcttatcgtgtgcaccagggt |
| | tacgaccatcgtggcgtagcgctctccgcc |
| | ggtgttcagcggatggtgcgctccgacctc |
| | gcatcttctggcgtgatgttctccattgat |
| | accgaatctggctttgaccaggtggtgttt |
| | atcacttccgcatggggccttggtgaaatg |
| | gtcgtgcagggtgcggttaacccggatgag |
| | ttttatgtgcataaaccgacacttgcggcg |
| | aatcgcccggctattgtgcgccgcaccatg |
| | gggtcgaaaaaaatccgcatggtttacgcg |
| | ccgacccaggagcacggcaagcaggttaaa |
| | atcgaagacgtaccgcaggaacagcgtgac |
| | atcttctcgctgaccaacgaagaagtgcag |
| | gaactggcaaaacaggccgtacaaattgag |
| | aaacactacggtcgcccgatggatattgag |
| | tgggcgaaagatggccacaccggcaaactg |
| | ttcattgtgcaggcgcgtccggaaaccgtg |
| | cgctcacgcggtcaggtcatggagcgttat |
| | acgctgcattcacagggtaagattatcgcc |
| | gaaggccgtgctatcggtcatcgcatcggc |
| | gcgggtccggtgaaagtcatccatgacatc |
| | agcgaaatgaaccgcatcgaacctggcgac |
| | gtgctggttactgacatgaccgacccggac |
| | tgggaaccgatcatgaagaaagcatctgcc |
| | atcgtcaccaaccgtggcggtcgtacctgt |
| | cacgcggcgatcatcgctcgtgaactcggc |
| | attccggcggtagtgggctgtggtgatgca |
| | acagaacggatgaaagacggcgagaacgtc |
| | actgtttcttgtgccgaaggtgataccggt |
| | tacgtctatgcggagttgctggaatttagc |
| | gtgaaaagctccagcgtagaaacgatgccg |
| | gacctgccgttgaaggtgatgatgaacgtc |
| | ggtaacccggaccgtgctttcgacttcgcc |
| | tgcctgccgaacgaaggcgtgggccttgcg |
| | cgtctggaatttatcatcaaccgtatgatt |
| | ggcgtccacccacgcgcactgcttgagttt |
| | gacgatcaggaaccgcagttgcaaaacgaa |
| | atccgcgagatgatgaaaggttttgattct |
| | ccgcgtgaattttacgttggtcgtctgact |
| | gaagggatcgcgacgctgggtgccgcgttt |
| | tatccgaagcgcgtcattgtccgtctctct |
| | gattttaaatcgaacgaatatgccaatctg |
| | gtcggtggtgagcgttacgagccagatgaa |
| | gagaacccgatgctcggcttccgtggcgcg |
| | ggccgctatgtttccgacagcttccgcgac |
| | tgcttcgcgctggagtgtgaagcagtgaaa |
| | cgtgtgcgcaacgacatggggctgactaac |
| | gttgagatcatgatcccgttcgtgcgtacc |
| | gttgatcaggcgaaagcggtggttgaggaa |
| | ctggcgcatcaggggctgaaacgtggtgag |
| | aacgggctgaaaatcatcatgatgtgtgaa |
| | attccgtccaacgccttgctggcagagcag |
| | ttcctggaatatttcgacggcttctcaatt |
| | ggctcaaacgacatgacgcagctggcgctc |
| | ggtctggatcgtgactccggcgtggtgtct |
| | gaactgttcgatgagcgcaacgatgcggtg |
| | aaagcactgctgtcgatggcgattcgtgcc |
| | gcgaagaaacagggcaaatatgtcgggatt |
| | tgcggtcagggtccatccgaccacgaagac |
| | tttgctgcatggttgatggaagaggggatc |
| | gatagcctgtctctgaacccggacaccgtg |
| | gtgcaaacctggttaagcctggctgaactg |
| | aagaaataa |
| dld nucleic acid | ATGTCTTCCATGACAACAACTGATAATAAA |
| sequence (SEQ ID | GCCTTTTTGAATGAACTTGCCCGTCTGGTC |
| NO: 13) | GGTCATTCACACCTGCTCACCGATCCCGCA |
| | AAAACGGCCCGCTATCGCAAGGGCTTCCGT |
| | TCTGGTCAGGGCGACGCGCTTGCTGTCGTT |
| | TTCCCTGGCTCACTACTAGAATTGTGGCGG |
| | GTGCTGAAAGCCTGCGTCACCGCTGACAAA |
| | ATTATTCTGATGCAGGCTGCCAATACAGGC |
| | CTGACCGAAGGATCGACGCCAAACGGTAAC |
| | GATTATGATCGCGATATCGTGATCATCAGC |
| | ACCCTGCGTCTCGACAAGCTGCACGTTCTC |
| | GGCAAGGGCGAACAAGTGCTGGCCTATCCG |
| | GGCACCACGCTCTATTCACTGGAAAAAGCC |
| | CTCAAACCGCTGGGACGCGAACCGCACTCA |
| | GTGATTGGATCATCGTGTATAGGCGCATCG |
| | GTCATCGGCGGTATTTGTAACAACTCGGGC |
| | GGCTCGCTGGTGCAACGTGGCCCGGCGTAT |
| | ACCGAAATGTCATTATTCGCGCGTATAAAT |
| | GAAGACGGCAAACTGACGCTGGTGAACCAT |
| | CTGGGGATTGATCTGGGCGAAACGCCGGAG |
| | CAGATCCTTAGCAAGCTGGATGACGATCGC |
| | ATCAAAGATGACGATGTGCGTCACGATGGT |
| | CGTCACGCCCACGATTATGACTATGTCCAC |
| | CGCGTTCGTGATATTGAAGCCGACACGCCC |
| | GCACGTTATAACGCCGATCCGGATCGGTTA |
| | TTTGAATCTTCTGGTTGCGCAGGTAAGCTG |
| | GCCGTCTTTGCGGTACGTCTTGATACCTTC |
| | GAAGCGGAAAAAAATCAGCAGGTGTTTTAT |
| | ATCGGCACCAACCAGCCGGAAGTGCTGACC |
| | GAAATCCGCCGTCATATTCTGGCTAATTTC |
| | GAAAATCTGCCGGTTGCCGGGGAATATATG |
| | CACCGGGATATCTACGATATTGCGGAAAAA |
| | TACGGCAAAGACACCTTCCTGATGATTGAT |
| | AAGTTAGGCACCGACAAGATGCCGTTCTTC |
| | TTTAATCTCAAGGGACGCACCGATGCGATG |
| | CTGGAGAAAGTGAAATTCTTCCGTCCGCAT |
| | TTTACCGACCGTGCAATGCAAAAATTCGGT |
| | CACCTGTTCCCCAGCCATTTACCGCCGCGC |
| | ATGAAAAACTGGCGCGATAAATACGAGCAT |
| | CATCTGCTGTTAAAAATGGCGGGCGATGGC |
| | GTCGGTGAAGCCAAATCGTGGCTAGTGGAT |
| | TATTTCAAACAGGCCGAGGGCGATTTCTTT |
| | GTCTGTACGCCGGAGGAAGGCAGCAAAGCG |
| | TTTTTACACCGTTTCGCCGCTGCGGGCGCA |
| | GCAATTCGTTATCAGGCTGTGCATTCCGAT |
| | GAAGTCGAAGACATTCTGGCGCTGGATATC |
| | GCTCTGCGGCGTAACGACACCGAATGGTAT |
| | GAGCATTTACCGCCGGAGATCGACAGCCAG |
| | CTGGTGCACAAGCTCTATTATGGCCATTTT |
| | ATGTGCTATGTCTTCCATCAGGATTACATC |
| | GTGAAAAAAGGCGTGGATGTGCATGTGTTG |
| | AAAGAACAGATGCTGGAACTGCTACAGCAG |
| | CGCGGCGCGCAATACCCTGCCGAGCATAAC |
| | GTCGGTCATTTGTATAAAGCACCGGAAACG |
| | TTGCAGAAGTTTTATCGCGAGAACGATCCG |
| | ACCAACAGTATGAATCCGGGGATCGGTAAA |
| | ACCAGTAAGCGGAAAAACTGGCAGGAAGTG |
| | GAGTAA |
| lldD nucleic acid | atgattatttccgcagccagcgattatcgc |
| sequence (SEQ ID | gccgcagcgcaacgcattctgccgccgttc |
| NO: 14) | ctgttccactatatggatgggggggcatat |
| | tctgaatacacgctgcgccgcaacgtggaa |
| | gatttgtcagaagtggcgctgcgccagcgt |
| | attctgaaaaacatgtctgacttaagcctg |
| | gaaacgacgctgtttaatgagaaattgtcg |
| | atgccggtggcgctaggtccggtaggtttg |
| | tgtggcatgtatgcgcgacgcggcgaagtt |
| | caggctgccaaagcagcagatgcgcatggc |
| | attccgtttactctctcgacggtttccgtt |
| | tgcccgattgaagaagtggctccggctatc |
| | aaacgtccgatgtggttccagctttatgtg |
| | ctgcgcgatcgcggctttatgcgtaacgcc |
| | ctggagcgagcaaaagccgcgggttgttcg |
| | acgctggttttcaccgtggatatgccaacg |
| | ccgggagcgcgttatcgtgatgcgcattct |
| | gggatgagcggcccgaacgcggcaatgcgc |
| | cgctacttgcaggcggtgacgcatccgcaa |
| | tgggcgtgggatgtgggcctgaacggtcgt |
| | ccgcatgatttaggtaatatctcggcttac |
| | ctcggcaaaccaaccggactggaagattac |
| | atcggctggctggggaataacttcgatccg |
| | tccatctcatggaaagaccttgagtggatc |
| | cgcgatttctgggatggcccgatggtgatc |
| | aaagggatcctcgatccggaagatgcgcgc |
| | gatgcagtacgttttggtgctgatggaatt |
| | gtggtttctaaccacggtggccgccagtta |
| | gatggcgtactctcttctgctcgtgcactg |
| | cctgctattgcggatgcggtgaaaggtgat |
| | atcgccattctggcggatagcggaatacgt |
| | aacgggcttgatgtcgtgcgtatgattgcg |
| | ctcggtgccgacaccgtactgctgggtcgt |
| | gctttcctgtatgcactggcaacagcgggc |
| | caggcgggtgtagctaatctgctaaatctg |
| | atcgaaaaagagatgaaagtggcgatgacg |
| | ctgactggcgcgaaatcgattagcgaaatt |
| | acgcaagattcgctggtgcaggggctgggt |
| | aaagagttgcctgcggcactggctccaatg |
| | gcgaaagggaatgcagcttaa |
| mgsA | Atggaactgacgactcgcactttaccttcg |
| (methylglyoxyl | cggaaacatattgcgctggtggcacacgat |
| synthetase) | cactgcaaacaaatgctgatgagctgggtg |
| nucleic acid | gaacggcatcaaccgttactggaacaacac |
| sequence | gtactgtatgcaacaggcactaccggtaac |
| (SEQ ID | ttaatttcccgcgcgaccggcatgaacgtc |
| NO: 30) | aacgcgatgttgagtggcccaatggggggt |
| | gaccagcaggttggcgcattgatctcagaa |
| | gggaaaattgatgtattgattttcttctgg |
| | gacccactaaacgccgtgccgcacgaccct |
| | gacgtgaaagccttgctgcgtctggcgacg |
| | gtatggaacattccggttgccaccaacgtg |
| | gcaacggcagacttcattatccagtcgccg |
| | catttcaacgacgcggtcgatattctgatc |
| | cccgattatcagcgttatctcgcggaccgt |
| | ctgaagtaa |
| frdB | Atggctgagatgaaaaacctgaaaattgag |
| (fumarase | gtggtgcgctataacccggaagtcgatacc |
| reductase subunit) | gcaccgcatagcgcattctatgaagtgcct |
| nucleic acid | tatgacgcaactacctcattactggatgcg |
| sequence | ctgggctacatcaaagacaacctggcaccg |
| (SEQ ID | gacctgagctaccgctggtcctgccgtatg |
| NO: 32) | gcgatttgtggctcctgcggcatgatggtt |
| | aacaacgtgccaaaactggcatgtaaaacc |
| | ttcctgcgtgattacaccgacggtatgaag |
| | gttgaagcgttagctaacttcccgattgaa |
| | cgcgatctggtggtcgatatgactcacttc |
| | atcgaaagtctggaagcgatcaaaccgtac |
| | atcatcggcaactcccgcaccgcggatcag |
| | ggtactaacatccagaccccggcgcagatg |
| | gcgaagtatcaccagttctccggttgcatc |
| | aactgtggtctgtgctacgccgcgtgcccg |
| | cagtttggcctgaacccagagttcatcggt |
| | ccggctgccattacgctggcgcatcgttat |
| | aacgaagatagccgcgaccacggtaagaag |
| | gagcgtatggcgcagttgaacagccagaac |
| | ggcgtatggagctgtactttcgtgggctac |
| | tgctccgaagtctgcccgaaacacgtcgat |
| | ccggctgcggccattcagcagggcaaagta |
| | gaaagttcgaaagactttcttatcgcgacc |
| | ctgaaaccacgctaa |
| frdC | atgacgactaaacgtaaaccgtatgtacgg |
| (fumarase | ccaatgacgtccacctggtggaaaaaattg |
| reductase subunit) | ccgttttatcgcttttacatgctgcgcgaa |
| nucleic acid | ggcacggcggttccggctgtgtggttcagc |
| sequence | attgaactgattttcgggctgtttgccctg |
| (SEQ ID | aaaaatggcccggaagcctggggggattcg |
| NO: 34) | tcgactttttacaaaacccggttatcgtga |
| | tcattaacctgatcactctggcggcagccc |
| | tgctgcacaccaaaacctggtttgagctgg |
| | caccaaaagcagccaatatcattgtaaaag |
| | acgaaaaaatgggaccagagccaattatca |
| | aaagtctctgggcggtaactgtggttgcca |
| | ccatcgtaatcctgtttgttgccctgtact |
| | ggtaa |

Table 2 lists the amino acid sequences for the nucleic acid sequences set forth in Table 1.

TABLE 2
Amino Acid Sequences

| Description | Sequence |
|---|---|
| LdhA amino | MKLAVYSTKQYDKKYLQQVNESFGFELEFF |
| acid | DFLLTEKTAKTANGCEAVCIFVNDDGSRPV |
| sequence | LEELKKHGVKYIALRCAGFNNVDLDAAKEL |
| (SEQ ID | GLKVVRVPAYDPEAVAEHAIGMMMTLNRRI |
| NO: 15) | HRAYQRTRDANFSLEGLTGFTMYGKTAGVI |
| | GTGKIGVAMLRILKGFGMRLLAFDPYPSAA |
| | ALELGVEYVDLPTLFSESDVISLHCPLTPE |
| | NYHLLNEAAFEQMKNGVMIVNTSRGALIDS |
| | QAAIEALKNQKIGSLGMDVYENERDLFFED |
| | KSNDVIQDDVFRRLSACHNVLFTGHQAFLT |
| | AEALTSISQTTLQNLSNLEKGETCPNELV |
| LdhL amino | MKKVNRIAVVGTGAVGTSYCYAMINQGVAE |
| acid | ELVLIDINEAKAEGEAMDLNHGLPFAPTPT |
| sequence | RVWKGDYSDCGTADLVVITAGSPQKPGETR |
| (SEQ ID | LDLVAKNAKIFKGMIKSIMDSGFNGIFLVA |
| NO: 16) | SNPVDILTYVTWKESGLPKEHVIGSGTVLD |
| | SARLRNSLSAHFGIDPRNVHAAIIGEHGDT |
| | ELPVWSHTTIGYDTIESYLQKGTIDQKTLD |
| | DIFVNTRDAAYHIIERKGATFYGIGMSLTR |
| | ITRAILNNENSVLTVSAFLEGQYGNSDVYI |
| | GVPAVINRQGVREVVEIELNDKEQEQFSHS |
| | VKVLKETMAPVL |
| pta amino | MLIPTGTSVGLTSVSLGVIRAMERKGVRLS |
| acid | VFKPIAQPRTGGDAPDQTTTIVRANSSTTT |
| sequence | AAEPLKMSYVEGLLSSNQKDVLMEEIIANY |
| (SEQ ID | HANTKDAEVVLVEGLVPTRKHQFAQSLNYE |
| NO: 17) | IAKTLNAEIVFVMSQGTDTPEQLKERIELT |
| | RNSFGGAKNTNITGVIVNKLNAPVDEQGRT |
| | RPDLSEIFDDSTKAKVNNVDPAKLQESSPL |
| | PVLGAVPWSFDLIATRAIDMARHLNATIIN |
| | EGDINTRRVKSVTFCARSIPHMLEHFRAGS |
| | LLVTSADRPDVLVAACLAAMNGVEIGALLL |
| | TGGYEMDARISKLCERAFATGLPVFMVNTN |
| | TWQTSLSLQSFNLEVPVDDHERIEKVQEYV |
| | ANYINADWIDSLTATSERSRRLSPPAFRYQ |
| | LTELARKAGKRIVLPEGDEPRTVKAAAICA |
| | ERGIATCVLLGNPAEINRVAASQGVELGAG |
| | IEIVDPEVVRENYVGRLVELRKNKGMTETV |
| | AREQLEDNVVLGTLMLEQDEVDGLVSGAVH |
| | TTANTIRPPLQLIKTAPGSSLVSSVFFMLL |
| | PEQVYVYGDCAINPDPTAEQLAEIAIQSAD |
| | SAAAFGIEPRVAMLSYSTGTSGAGSDVEKV |
| | REATRLAQEKRPDLMIDGPLQYDAAVMADV |
| | AKSKAPNSPVAGRATVFIFPDLNTGNTTYK |
| | AVQRSADLISIGPMLQGMRKPVNDLSRGAL |
| | VDDIVYTIALTAIQSAQQQ |
| pflB amino | MSELNEKLATAWEGFTKGDWQNEVNVRDFI |
| acid | QKNYTPYEGDESFLAGATEATTTLWDKVME |
| sequence | GVKLENRTHAPVDFDTAVASTITSHDAGYI |
| (SEQ ID | NKQLEKIVGLQTEAPLKRALIPFGGIKMIE |
| NO: 18) | GSCKAYNRELDPMIKKIFTEYRKTHNQGVF |
| | DVYTPDILRCRKSGVLTGLPDAYGRGRIIG |
| | DYRRVALYGIDYLMKDKLAQFTSLQADLEN |
| | GVNLEQTIRLREEIAEQHRALGQMKEMAAK |
| | YGYDISGPATNAQEAIQWTYFGYLAAVKSQ |
| | NGAAMSFGRTSTFLDVYIERDLKAGKITEQ |
| | EAQEMVDHLVMKLRMVRFLRTPEYDELFSG |
| | DPIWATESIGGMGLDGRTLVTKNSFRFLNT |
| | LYTMGPSPEPNMTILWSEKLPLNFKKFAAK |
| | VSIDTSSLQYENDDLMRPDFNNDDYAIACC |
| | VSPMIVGKQMQFFGARANLAKTMLYAINGG |
| | VDEKLKMQVGPKSEPIKGDVLNYDEVMERM |
| | DHFMDWLAKQYITALNIIHYMHDKYSYEAS |
| | LMALHDRDVIRTMACGIAGLSVAADSLSAI |
| | KYAKVKPIRDEDGLAIDFEIEGEYPQFGNN |
| | DPRVDDLAVDLVERFMKKIQKLHTYRDAIP |
| | TQSVLTITSNVVYGKKTGNTPDGRRAGAPF |
| | GPGANPMHGRDQKGAVASLTSVAKLPFAYA |
| | KDGISYTFSIVPNALGKDDEVRKTNLAGLM |
| | DGYFHHEASIEGGQHLNVNVMNREMLLDAM |
| | ENPEKYPQLTIRVSGYAVRFNSLTKEQQQD |
| | VITRTFTQSM |
| ackA amino | MSSKLVLVLNCGSSSLKFAIIDAVNGEEYL |
| acid | SGLAECFHLPEARIKWKMDGNKQEAALGAG |
| sequence | AAHSEALNFIVNTILAQKPELSAQLTAIGH |
| (SEQ ID | RIVHGGEKYTSSVVIDESVIQGIKDAASFA |
| NO: 19) | PLHNPAHLIGIEEALKSFPQLKDKNVAVFD |
| | TAFHQTMPEESYLYALPYNLYKEHGIRRYG |
| | AHGTSHFYVTQEAAKMLNKPVEELNIITCH |
| | LGNGGSVSAIRNGKCVDTSMGLTPLEGLVM |
| | GTRSGDIDPAIIFHLHDTLGMSVDAINKLL |
| | TKESGLLGLTEVTSDCRYVEDNYATKEDAK |
| | RAMDVYCHRLAKYIGAYTALMDGRLDAVVF |
| | TGGIGENAAMVRELSLGKLGVLGFEVDHER |
| | NLAARFGKSGFINKEGTRPAVVIPTNEELV |
| | IAQDASRLTA |
| adhE amino | MAVTNVAELNALVERVKKAQREYASFTQEQ |
| acid | VDKIFRAAALAAADARIPLAKMAVAESGMG |
| sequence | IVEDKVIKNHFASEYIYNAYKDEKTCGVLS |
| (SEQ ID | EDDTFGTITIAEPIGIICGIVPTTNPTSTA |
| NO: 20) | IFKSLISLKTRNAIIFSPHPRAKDATNKAA |
| | DIVLQAAIAAGAPKDLIGWIDQPSVELSNA |
| | LMHHPDINLILATGGPGMVKAAYSSGKPAI |
| | GVGAGNTPVVIDETADIKRAVASVLMSKTF |
| | DNGVICASEQSVVVVDSVYDAVRERFATHG |
| | GYLLQGKELKAVQDVILKNGALNAAIVGQP |
| | AYKIAELAGFSVPENTKILIGEVTVVDESE |
| | PFAHEKLSPTLAMYRAKDFEDAVEKAEKLV |
| | AMGGIGHTSCLYTDQDNQPARVSYFGQKMK |
| | TARILINTPASQGGIGDLYNFKLAPSLTLG |
| | CGSWGGNSISENVGPKHLINKKTVAKRAEN |
| | MLWHKLPKSIYFRRGSLPIALDEVITDGHK |
| | RALIVTDRFLFNNGYADQITSVLKAAGVET |
| | EVFFEVEADPTLSIVRKGAELANSFKPDVI |
| | IALGGGSPMDAAKIMWVMYEHPETHFEELA |
| | LRFMDIRKRIYKFPKMGVKAKMIAVTTTSG |
| | TGSEVTPFAVVTDDTTGQKYPLADYALTPD |
| | MAIVDANLVMDMPKSLCAFGGLDAVTHAME |
| | AYVSVLASEFSDGQALQALKLLKEYLPASY |
| | HEGSKNPVARERVHSAATIAGIAFANAFLG |
| | VCHSMAHKLGSQFHIPHGLANALLICNVIR |
| | YNANDNPTKQTAFSQYDRPQARRRYAEIAD |
| | HLGLSAPGDRTAAKIEKLLAWLETLKAELG |
| | IPKSIREAGVQEADFLANVDKLSEDAFDDQ |
| | CTGANPRYPLISELKQILLDTYYGRDYVEG |
| | ETAAKKEAAPAKAEKKAKKSA |
| pfkA amino | MCKKTSGNRFHFAFQSSEVVMIKKIGVLTS |
| acid | GGDAPGMNAAIRGVVRSALTEGLEVMGIYD |
| sequence | GYLGLYEDRMVQLDRYSVSDMINRGGTFLG |
| (SEQ ID | SARFPEFRDENIRAVAIENLKKRGIDALVV |
| NO: 21) | IGGDGSYMGAMRLTEMGFPCIGLPGTIDND |
| | IKGTDYTIGFFTALSTVVEAIDRLRDTSSS |
| | HQRISVVEVMGRYCGDLTLAAAIAGGCEFV |
| | VVPEVEFSREDLVNEIKAGIAKGKKHAIVA |
| | ITEHMCDVDELAHFIEKETGRETRATVLGH |
| | IQRGGSPVPYDRILASRMGAYAIELLLAGY |
| | GGRCVGIQNEQLVHHDIIDAIENMKRPFKG |
| | DWLDCAKKLY |
| frdA amino | VQTFQADLAIVGAGGAGLRAAIAAAQANPN |
| acid | AKIALISKVYPMRSHTVAAEGGSAAVAQDH |
| sequence | DSFEYHFHDTVAGGDWLCEQDVVDYFVHHC |
| (SEQ ID | PTEMTQLELWGCPWSRRPDGSVNVRRFGGM |
| NO: 22) | KIERTWFAADKTGFHMLHTLFQTSLQFPQI |
| | QRFDEHFVLDILVDDGHVRGLVAMNMMEGT |
| | LVQIRANAVVMATGGAGRVYRYNTNGGIVT |
| | GDGMGMALSHGVPLRDMEFVQYHPTGLPGS |
| | GILMTEGCRGEGGILVNKNGYRYLQDYGMG |
| | PETPLGEPKNKYMELGPRDKVSQAFWHEWR |
| | KGNTISTPRGDVVYLDLRHLGEKKLHERLP |
| | FICELAKAYVGVDPVKEPIPVRPTAHYTMG |
| | GIETDQNCETRIKGLFAVGECSSVGLHGAN |
| | RLGSNSLAELVVFGRLAGEQATERAATAGN |
| | GNEAAIEAQAAGVEQRLKDLVNQDGGENWA |
| | KIRDEMGMAMEEGCGIYRTPELMQKTIDKL |
| | AELQERFKRVRITDTSSVFNTDLLYTIELG |
| | HGLNVAECMAHSAMARKESRGAHQRLDEGC |
| | TERDDVNFLKHTLAFRDADGTTRLEYSDVK |
| | ITTLPPAKRVYGGEADAADKAEAANKKEKA |
| | NG |
| poxB amino | MKQTVAAYIAKTLESAGVKRIWGVTGDSLN |
| acid | GLSDSLNRMGTIEWMSTRHEEVAAFAAGAE |
| sequence | AQLSGELAVCAGSCGPGNLHLINGLFDCHR |
| (SEQ ID | NHVPVLAIAAHIPSSEIGSGYFQETHPQEL |
| NO: 23) | FRECSHYCELVSSPEQIPQVLAIAMRKAVL |
| | NRGVSVVVLPGDVALKPAPEGATTHWYHAP |
| | QPVVTPEEEELRKLAQLLRYSSNIALMCGS |
| | GCAGAHKELVEFAGKIKAPIVHALRGKEHV |
| | EYDNPYDVGMTGLIGFSSGFHTMMNADTLV |
| | LLGTQFPYRAFYPTDAKIIQIDINPASIGA |
| | HSKVDMALVGDIKSTLRALLPLVEEKTDRK |
| | FLDKALEDYRDARKGLDDLAKPSEKAIHPQ |
| | YLAQQISHFAADDAIFTCDVGTPTVWAARY |
| | LKMNGKRRLLGSFNHGSMANAMPQALGAQA |
| | TEPERQVVAMCGDGGFSMLMGDFLSVMQMK |
| | LPVKIIVFNNSVLGFVAMEMKAGGYLTDGT |
| | ELHDTNFARIAEACGITGIRVEKASEIDEA |
| | LQRAFSIDGPVLVDVVVAKEELAIPPQIKL |
| | EQAKGFSLYMLRAIISGRGDEVIELAKTNW |
| | LR |
| pps amino | MSNNGSSPLVLWYNQLGMNDVDRVGGKNAS |
| acid | LGEMITNLSGMGVSVPNGFATTADAFNQFL |
| sequence | DQSGVNQRIYELLDKTDIDDVTQLAKAGAQ |
| (SEQ ID | IRQWIIDTPFQPELENAIREAYAQLSADDE |
| NO: 24) | NASFAVRSSATAEDMPDASFAGQQETFLNV |
| | QGFDAVLVAVKHVFASLFNDRAISYRVHQG |
| | YDHRGVALSAGVQRMVRSDLASSGVMFSID |
| | TESGFDQVVFITSAWGLGEMVVQGAVNPDE |
| | FYVHKPTLAANRPAIVRRTMGSKKIRMVYA |
| | PTQEHGKQVKIEDVPQEQRDIFSLTNEEVQ |
| | ELAKQAVQIEKHYGRPMDIEWAKDGHTGKL |
| | FIVQARPETVRSRGQVMERYTLHSQGKIIA |
| | EGRAIGHRIGAGPVKVIHDISEMNRIEPGD |
| | VLVTDMTDPDWEPIMKKASAIVTNRGGRTC |
| | HAAIIARELGIPAVVGCGDATERMKDGENV |
| | TVSCAEGDTGYVYAELLEFSVKSSSVETMP |
| | DLPLKVMMNVGNPDRAFDFACLPNEGVGLA |
| | RLEFIINRMIGVHPRALLEFDDQEPQLQNE |
| | IREMMKGFDSPREFYVGRLTEGIATLGAAF |
| | YPKRVIVRLSDFKSNEYANLVGGERYEPDE |
| | ENPMLGFRGAGRYVSDSFRDCFALECEAVK |
| | RVRNDMGLTNVEIMIPFVRTVDQAKAVVEE |
| | LAHQGLKRGENGLKIIMMCEIPSNALLAEQ |
| | FLEYFDGFSIGSNDMTQLALGLDRDSGVVS |
| | ELFDERNDAVKALLSMAIRAAKKQGKYVGI |
| | CGQGPSDHEDFAAWLMEEGIDSLSLNPDTV |
| | VQTWLSLAELKK |
| dld amino | MSSMTTTDNKAFLNELARLVGHSHLLTDPA |
| acid | KTARYRKGFRSGQGDALAVVFPGSLLELWR |
| sequence | VLKACVTADKIILMQAANTGLTEGSTPNGN |
| (SEQ ID | DYDRDIVIISTLRLDKLHVLGKGEQVLAYP |
| NO: 25) | GTTLYSLEKALKPLGREPHSVIGSSCIGAS |
| | VIGGICNNSGGSLVQRGPAYTEMSLFARIN |
| | EDGKLTLVNHLGIDLGETPEQILSKLDDDR |
| | IKDDDVRHDGRHAHDYDYVHRVRDIEADTP |
| | ARYNADPDRLFESSGCAGKLAVFAVRLDTF |
| | EAEKNQQVFYIGTNQPEVLTEIRRHILANF |
| | ENLPVAGEYMHRDIYDIAEKYGKDTFLMID |
| | KLGTDKMPFFFNLKGRTDAMLEKVKFFRPH |
| | FTDRAMQKFGHLFPSHLPPRMKNWRDKYEH |
| | HLLLKMAGDGVGEAKSWLVDYFKQAEGDFF |
| | VCTPEEGSKAFLHRFAAAGAAIRYQAVHSD |
| | EVEDILALDIALRRNDTEWYEHLPPEIDSQ |
| | LVHKLYYGHFMCYVFHQDYIVKKGVDVHVL |
| | KEQMLELLQQRGAQYPAEHNVGHLYKAPET |
| | LQKFYRENDPTNSMNPGIGKTSKRKNWQEV |
| | E |
| UldD amino | MIISAASDYRAAAQRILPPFLFHYMDGGAY |
| acid | SEYTLRRNVEDLSEVALRQRILKNMSDLSL |
| sequence | ETTLFNEKLSMPVALGPVGLCGMYARRGEV |
| (SEQ ID | QAAKAADAHGIPFTLSTVSVCPIEEVAPAI |
| NO: 26) | KRPMWFQLYVLRDRGFMRNALERAKAAGCS |
| | TLVFTVDMPTPGARYRDAHSGMSGPNAAMR |
| | RYLQAVTHPQWAWDVGLNGRPHDLGNISAY |
| | LGKPTGLEDYIGWLGNNFDPSISWKDLEWI |
| | RDFWDGPMVIKGILDPEDARDAVRFGADGI |
| | VVSNHGGRQLDGVLSSARALPAIADAVKGD |
| | IAILADSGIRNGLDVVRMIALGADTVLLGR |
| | AFLYALATAGQAGVANLLNLIEKEMKVAMT |
| | LTGAKSISEITQDSLVQGLGKELPAALAPM |
| | AKGNAA |
| mgsA amino | MELTTRTLPSRKHIALVAHDHCKQMLMSWV |
| acid | ERHQPLLEQHVLYATGTTGNLISRATGMNV |
| sequence | NAMLSGPMGGDQQVGALISEGKIDVLIFFW |
| (SEQ ID | DPLNAVPHDPDVKALLRLATVWNIPVATNV |
| NO: 31) | ATADFIIQSPHFNDAVDILIPDYQRYLADR |
| | LK |
| frdB amino | MAEMKNLKIEVVRYNPEVDTAPHSAFYEVP |
| acid | YDATTSLLDALGYIKDNLAPDLSYRWSCRM |
| sequence | AICGSCGMMVNNVPKLACKTFLRDYTDGMK |
| (SEQ ID | VEALANFPIERDLVVDMTHFIESLEAIKPY |
| NO: 33) | IIGNSRTADQGTNIQTPAQMAKYHQFSGCI |
| | NCGLCYAACPQFGLNPEFIGPAAITLAHRY |
| | NEDSRDHGKKERMAQLNSQNGVWSCTFVGY |
| | CSEVCPKHVDPAAAIQQGKVESSKDFLIAT |
| | LKPR |
| frdC amino | MTTKRKPYVRPMTSTWWKKLPFYRFYMLRE |
| acid | GTAVPAVWFSIELIFGLFALKNGPEAWAGF |
| sequence | VDFLQNPVIVIINLITLAAALLHTKTWFEL |
| (SEQ ID | APKAANIIVKDEKMGPEPIIKSLWAVTVVA |
| NO: 35) | TIVILFVALYW |

In some embodiments, the recombinant bacteria comprise one or more nucleic acid sequence(s) of Table 1 (SEQ ID NO: 1-SEQ ID NO: 14) or a functional fragment thereof. In some embodiments, the recombinant bacteria comprise a nucleic acid sequence that, but for the redundancy of the genetic code, encodes the same polypeptide as SEQ ID NO: 15 and/or SEQ ID NO: 16 or a functional fragment thereof. In some embodiments, recombinant bacteria comprise a nucleic acid sequence that is at least about 80%, at least about 85%, at least about 90%, at least about 95%, or at least about 99% homologous to the DNA sequence of one or more nucleic acid sequence(s) of Table 1 (SEQ ID NO: 1-SEQ ID NO: 14) or a functional fragment thereof. In some embodiments, recombinant bacteria comprise a nucleic acid sequence that is at least about 80%, at least about 85%, at least about 90%, at least about 95%, or at least about 99% homologous to the DNA sequence of SEQ ID NO: 15 and/or SEQ ID NO: 16, or a nucleic acid sequence that, but for the redundancy of the genetic code, encodes the same polypeptide as SEQ ID NO: 15 and/or SEQ ID NO: 16 or a functional fragment thereof. In some embodiments, the recombinant bacteria comprise a polypeptide sequence of SEQ ID NO: 15 and/or SEQ ID NO: 16 or a functional fragment or variant thereof. In some embodiments, recombinant bacteria comprise a polypeptide sequence that is at least about 80%, at least about 85%, at least about 90%, at least about 95%, or at least about 99% homologous to the polypeptide sequence of SEQ ID NO: 15 and/or SEQ ID NO: 16 or a functional fragment thereof.

One of skill in the art would appreciate that additional genes and gene cassettes capable of producing the metabolite, e.g., D-lactate and/or L-lactate, are known in the art and may be expressed by the recombinant bacteria.

In some embodiments, the recombinant bacteria are capable of expressing any one or more of the gene or gene cassettes described herein and further comprise one or more antibiotic resistance circuits known in the art, e.g., ampicillin resistant.

In any of these embodiments, the gene encoding phosphate acetyltransferase (pta) may be deleted, mutated, or modified within the recombinant bacteria so as to diminish or obliterate its catalytic function producing acetate from acetyl-CoA. Also, in any of these embodiments, the gene encoding formate acetyltransferase 1 (pflB) or acetate kinase (ackA) may be deleted, mutated, or modified so as to inhibit the production of acetyl-CoA and acetate, respectively, from pyruvate. In any of these embodiments, the gene encoding aldehyde dehydrogenase (adhE) or phosphofructokinase (pfkA) may be deleted, mutated, or modified so as to inhibit the production of ethanol and fructose, respectively. In any of these embodiments, the gene encoding fumarate reductase flavoprotein subunit (frdA), pyruvate dehydrogenase (poxB), phosphoenolpyruvate synthase (pps), quinone-dependent D-lactate dehydrogenase (dld), methylglyoxyl synthetase (mgsA), fumarase reductase subunit (frdB), fumarase reductase subunit (frdC), and/or L-lactate dehydrogenase (lldD) may be deleted, mutated, or modified.

The gene or gene cassette for producing the metabolite may be expressed under the control of a promoter. The gene or gene cassette can be either directly or indirectly operably linked to a promoter. In some embodiments, the promoter is not operably linked with the gene or gene cassette in nature. In some embodiments, the gene or gene cassette is expressed under the control of a constitutive promoter. In another embodiment, the gene or gene cassette is expressed under the control of an inducible promoter. In some embodiments, the gene or gene cassette is expressed under the control of a promoter that is directly or indirectly induced by exogenous environmental conditions. In one embodiment, the gene or gene cassette is expressed under the control of a promoter that is directly or indirectly induced by low-oxygen or anaerobic conditions, wherein expression of the gene or gene cassette is activated under low-oxygen or anaerobic environments, such as the environment of the mammalian gut. In some embodiments, the gene or gene cassette is expressed under the control of an oxygen level-dependent promoter.

Examples of oxygen level-dependent transcription factors and corresponding promoters and/or regulatory regions include, but are not limited to, the fumarate and nitrate reductase regulator (FNR), the anaerobic arginine deiminiase and nitrate reductase regulator (ANR), and the dissimilatory nitrate respiration regulator (DNR). Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 3.

TABLE 3
Examples of transcription factors and
responsive genes and regulatory regions

| | Examples of responsive genes, promoters, |
|---|---|
| Transcription Factor | and/or regulatory regions: |
| FNR | nirB, ydfZ, pdhR, focA, ndH, hlyE, narK, narX, |
| | narG, yfiD, tdcD |
| ANR | arcDABC |
| DNR | norb, norC |

In certain embodiments, the bacterial cell comprises at least one gene, gene(s), or gene cassettes for producing the metabolite, e.g., D-lactate, which is expressed under the control of the fumarate and nitrate reductase regulator (FNR) promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive.

FNR-responsive promoter sequences are known in the art, and any suitable FNR-responsive promoter sequence(s) may be used in the recombinant bacteria. An exemplary FNR-responsive promoter sequences is provided in Table 4. Lowercase letters are ribosome binding sites.

TABLE 4
FNR Promoter Sequences

| FNR Responsive | |
|---|---|
| Promoter | Sequence |
| SEQ ID NO: 27 | AGTTGTTCTTATTGGTGGTGTTGC |
| | TTTATGGTTGCATCGTAGTAAATG |
| | GTTGTAACAAAAGCAATTTTTCCG |
| | GCTGTCTGTATACAAAAACGCCGC |
| | AAAGTTTGAGCGAAGTCAATAAAC |
| | TCTCTACCCATTCAGGGCAATATC |
| | TCTCTTggatccaaagtgaaCCCG |
| | C |

In one embodiment, the FNR responsive promoter comprises SEQ ID NO: 27. In another embodiment, the FNR responsive promoter has at least 90%, 91%, 92%, 93%, 94%, 95%, 96%, 97%, 98%, 99%, comprises or consists of SEQ ID NO:27.

In alternate embodiments, the recombinant bacteria comprising at least one gene, gene(s), or gene cassettes for producing the metabolite, e.g., D-lactate, is expressed under the control of an alternate oxygen level-dependent promoter, e.g., DNR (Trunk et al., Environ Microbiol. 2010; 12(6):1719-33) or ANR (Ray et al., FEMS Microbiol Lett. 1997; 156(2):227-32). In these embodiments, expression of the metabolite, e.g., D-lactate, is particularly activated in a low-oxygen or anaerobic environment, such as in the mammalian gut. In some embodiments, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene, gene(s), or gene cassettes for producing the metabolite, e.g., D-lactate, in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., BMC Genomics. 2011; 12:51). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the recombinant bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, as compared to the wild-type promoter under the same conditions. In some embodiments, the recombinant bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., J Biol Chem. 2006; 281(44):33268-75).

In some embodiments, the bacterial cells disclosed herein comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene, gene(s), or gene cassettes for producing the metabolites, e.g., D-lactate and/or L-lactate, are present on different plasmids. In some embodiments, the gene encoding the oxygen level-sensing transcriptional regulator and the gene, gene(s), or gene cassettes for producing the metabolite, e.g., D-lactate and/or L-lactate, are present on different plasmids. In some embodiments, the gene encoding the oxygen level-sensing transcriptional regulator and the gene, gene(s), or gene cassettes for producing the metabolite, e.g., D-lactate and/or L-lactate, are present on the same plasmid.

In some embodiments, the gene encoding the oxygen level-sensing transcriptional regulator is present on a chromosome. In some embodiments, the gene encoding the oxygen level-sensing transcriptional regulator and the gene, gene(s), or gene cassettes for producing the metabolite, e.g., D-lactate and/or L-lactate, are present on different chromosomes. In some embodiments, the gene encoding the oxygen level-sensing transcriptional regulator and the gene, gene(s), or gene cassettes for producing the metabolite, e.g., D-lactate and/or L-lactate, are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene, gene(s), or gene cassettes for producing the metabolite, e.g., D-lactate and/or L-lactate. In some embodiments, expression of the transcriptional regulator is controlled by the same promoter that controls expression of the gene, gene(s), or gene cassettes for producing the metabolite, e.g., D-lactate and/or L-lactate. In some embodiments, the transcriptional regulator and the metabolite, e.g., D-lactate and/or L-lactate, are divergently transcribed from a promoter region.

In certain embodiments, the bacterial cell comprises at least one gene, gene(s), or gene cassettes for producing the metabolite, e.g., D-lactate and/or L-lactate, which is expressed under the control of the temperature sensitive promoter PcI857. An exemplary PcI857 promoter sequences is provided in Table 5.

TABLE 5
Exemplary PcI857 promoter sequences

| Temperature | |
|---|---|
| Sensitive | |
| Promoter | |
| (Pc1857) | Sequence |
| PcI857 | TCAGCCAAACGTCTCTTCAG |
| (SEQ ID | GCCACTGACTAGCGATAACT |
| NO: 28) | TTCCCCACAACGGAACAACT |
| | CTCATTGCATGGGATCATTG |
| | GGTACTGTGGGTTTAGTGGT |
| | TGTAAAAACACCTGACCGCT |
| | ATCCCTGATCAGTTTCTTGA |
| | AGGTAAACTCATCACCCCCA |
| | AGTCTGGCTATGCAGAAATC |
| | ACCTGGCTCAACAGCCTGCT |
| | CAGGGTCAACGAGAATTAAC |
| | ATTCCGTCAGGAAAGCTTGG |
| | CTTGGAGCCTGTTGGTGCGG |
| | TCATGGAATTACCTTCAACC |
| | TCAAGCCAGAATGCAGAATC |
| | ACTGGCTTTTTTGGTTGTGC |
| | TTACCCATCTCTCCGCATCA |
| | CCTTTGGTAAAGGTTCTAAG |
| | CTTAGGTGAGAACATCCCTG |
| | CCTGAACATGAGAAAAAACA |
| | GGGTACTCATACTCACTTCT |
| | AAGTGACGGCTGCATACTAA |
| | CCGCTTCATACATCTCGTAG |
| | ATTTCTCTGGCGATTGAAGG |
| | GCTAAATTCTTCAACGCTAA |
| | CTTTGAGAATTTTTGTAAGC |
| | AATGCGGCGTTATAAGCATT |
| | TAATGCATTGATGCCATTAA |
| | ATAAAGCACCAACGCCTGAC |
| | TGCCCCATCCCCATCTTGTC |
| | TGCGACAGATTCCTGGGATA |
| | AGCCAAGTTCATTTTTCTTT |
| | TTTTCATAAATTGCTTTAAG |
| | GCGACGTGCGTCCTCAAGCT |
| | GCTCTTGTGTTAATGGTTTC |
| | TTTTTTGTGCTCAT |

In one embodiment, the pcI857 promoter sequence has at least 90%, 91%, 92%, 93%, 94%, 95%, 96%, 97%, 98%, 99%, comprises or consists of SEQ ID NO:28. In some embodiments, gene expression is further optimized by methods known in the art, e.g., by optimizing ribosomal binding sites and/or increasing mRNA stability.

In some embodiments, the gene or gene cassette for producing D-lactate and/or L-lactate is expressed under the control of an oxygen level-dependent promoter fused to a binding site for a transcriptional activator, e.g., CRP. CRP (cyclic AMP receptor protein or catabolite activator protein or CAP) plays a major regulatory role in bacteria by repressing genes responsible for the uptake, metabolism and assimilation of less favorable carbon sources when rapidly metabolizable carbohydrates, such as glucose, are present (Wu et al., Sci Rep. 2015; 5: 14921). This preference for glucose has been termed glucose repression, as well as carbon catabolite repression (Deutscher, Curr Opin Microbiol. 2008; 11(2):87-93; Gorke and Stülke, Nature Reviews Microbiology, 2008, 6: 954). In some embodiments, expression of the gene or gene cassette is controlled by an oxygen level-dependent promoter fused to a CRP binding site. In some embodiments, expression of the gene or gene cassette is controlled by a FNR promoter fused to a CRP binding site. In these embodiments, cyclic AMP binds to CRP when no glucose is present in the environment. This binding causes a conformational change in CRP, and allows CRP to bind tightly to its binding site. CRP binding then activates transcription of the gene or gene cassette by recruiting RNA polymerase to the FNR promoter via direct protein-protein interactions. In the presence of glucose, cyclic AMP does not bind to CRP and gene transcription is repressed. In some embodiments, an oxygen level-dependent promoter (e.g., a FNR-responsive promoter) fused to a binding site for a transcriptional activator is used to ensure that the gene or gene cassette is not expressed under anaerobic conditions when sufficient amounts of glucose are present, e.g., by adding glucose to growth media in vitro.

In some embodiments, the gene or gene cassette for producing the D-lactate and/or L-lactate is expressed under the control of an oxygen level-dependent promoter operably linked to a detectable product, e.g., GFP, and can be used to screen for mutants. In some embodiments, the oxygen level-dependent promoter is mutagenized, and mutants are selected based upon the level of detectable product, e.g., by flow cytometry, fluorescence-activated cell sorting (FACS) when the detectable product fluoresces. In some embodiments, one or more transcription factor binding sites is mutagenized to increase or decrease binding. In alternate embodiments, the wild-type binding sites are left intact and the remainder of the regulatory region is subjected to mutagenesis. In some embodiments, the mutant promoter is inserted into the recombinant bacteria to increase expression of the D-lactate and/or L-lactate molecule in low-oxygen conditions, as compared to wild type bacteria of the same subtype under the same conditions. In some embodiments, the oxygen level-sensing transcription factor and/or the oxygen level-dependent promoter is a synthetic, non-naturally occurring sequence.

In some embodiments, one or more of the genes in a gene cassette for producing D-lactate and/or L-lactate, is mutated to increase expression of said molecule in low oxygen conditions, as compared to unmutated bacteria of the same subtype under the same conditions.

In one embodiment, the bacterial cell comprises a heterologous ldhA gene and/or ldhL gene. In some embodiments, the disclosure provides a bacterial cell that comprises a heterologous ldhA gene and/or ldhL gene operably linked to a first promoter. In one embodiment, the first promoter is an inducible promoter. In one embodiment, the bacterial cell comprises an ldhA gene and/or ldhL gene from a different organism, e.g., a different species of bacteria. In another embodiment, the bacterial cell comprises more than one copy of a native gene encoding an ldhA gene and/or ldhL gene. In yet another embodiment, the bacterial cell comprises at least one native gene encoding an ldhA gene and/or ldhL gene, as well as at least one copy of an ldhA gene and/or ldhL gene from a different organism, e.g., a different species of bacteria. In one embodiment, the bacterial cell comprises at least one, two, three, four, five, or six copies of a gene encoding an ldhA gene and/or ldhL gene. In one embodiment, the bacterial cell comprises multiple copies of a gene or genes encoding an ldhA gene and/or ldhL gene.

Multiple distinct ldhA genes and/or ldhL gene are known in the art. In some embodiments, an ldhA gene and/or ldhL gene is encoded by a gene cassette derived from a bacterial species. In some embodiments, an ldhA gene and/or ldhL gene is encoded by a gene derived from a non-bacterial species. In some embodiments, an ldhA gene and/or ldhL gene is encoded by a gene derived from a eukaryotic species, e.g., a fungi. In one embodiment, the gene encoding the ldhA gene and/or ldhL gene is derived from an organism of the genus or species that includes, but is not limited to, Escherichia coli, Bacillus coagulans, Clostridium propionicum, Megasphaera elsdenii, or Prevotella ruminicola.

In one embodiment, the ldhA gene and/or ldhL gene has been codon-optimized for use in the engineered bacterial cell. In one embodiment, the ldhA gene and/or ldhL gene has been codon-optimized for use in Escherichia coli. In another embodiment, the ldhA gene and/or ldhL gene has been codon-optimized for use in Lactococcus. When the ldhA gene and/or ldhL gene is expressed in the engineered bacterial cells, the bacterial cells produce more ldhA and/or ldhL than unmodified bacteria of the same bacterial subtype under the same conditions (e.g., culture or environmental conditions). Thus, the recombinant bacteria comprising a heterologous ldhA gene cassette and/or ldhL gene cassette may be used to generate D-lactate and/or L-lactate to treat autoimmune and inflammatory disease or disorders, such as multiple sclerosis.

The present disclosure further comprises genes encoding functional fragments of D-lactate biosynthesis enzymes and/or L-lactate biosynthesis enzymes or functional variants of an D-lactate biosynthesis enzyme and/or L-lactate biosynthesis enzymes. As used herein, the term “functional fragment thereof” or “functional variant thereof” relates to an element having qualitative biological activity in common with the wild-type enzyme from which the fragment or variant was derived. For example, a functional fragment or a functional variant of a mutated D-lactate biosynthesis enzyme and/or L-lactate biosynthesis enzymes is one which retains essentially the same ability to synthesize D-lactate and/or L-lactate as the D-lactate biosynthesis enzyme and/or L-lactate biosynthesis enzymes from which the functional fragment or functional variant was derived. For example a polypeptide having D-lactate biosynthesis enzyme and/or L-lactate biosynthesis enzyme activity may be truncated at the N-terminus or C-terminus, and the retention of D-lactate biosynthesis enzyme and/or L-lactate biosynthesis enzymes activity assessed using assays known to those of skill in the art, including the exemplary assays provided herein. In one embodiment, the engineered bacterial cell comprises a heterologous gene encoding a D-lactate biosynthesis enzyme functional variant and/or L-lactate biosynthesis enzyme functional variant. In another embodiment, the engineered bacterial cell comprises a heterologous gene encoding a D-lactate biosynthesis enzyme functional fragment and/or L-lactate biosynthesis enzyme functional fragment.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The present disclosure encompasses D-lactate biosynthesis enzymes and/or L-lactate biosynthesis enzymes comprising amino acids in its sequence that are substantially the same as an amino acid sequence described herein Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Ile, Asn, Gln, Phe, Cys, Pro, Trp, Tyr, Val).

In some embodiments, an D-lactate biosynthesis enzyme and/or L-lactate biosynthesis enzyme is mutagenized; mutants exhibiting increased activity are selected; and the mutagenized gene encoding the D-lactate biosynthesis enzyme and/or L-lactate biosynthesis enzyme is isolated and inserted into the bacterial cell of the disclosure. The gene comprising the modifications described herein may be present on a plasmid or chromosome.

In one embodiment, the D-lactate biosynthesis gene and/or L-lactate biosynthesis gene is from Bacillus, Escherichia, Clostridium, Megasphaera, Prevotella, Lactobacillus, Carnobacterium, Lactococcus, Streptococcus, Enterococcus, Vagococcus, Leuconostoc, Oenococcus, Pediococcus, Tetragonococcus, Aerococcus, and Weissella, e.g., Escherichia coli, Bacillus coagulans, Clostridium propionicum, Megasphaera elsdenii, Prevotella ruminicola, Lactobacillus acidophilus, Lactobacillus gasseri, Lactobacillus delbrueckii subsp. bulgaricus, Lactobacillus fermentum, Lactobacillus lactis, Lactobacillus brevis, Lactobacillus helveticus, Lactobacillus plantarum and Lactobacillus reuteri. In one embodiment, the D-lactate biosynthesis gene and/or the L-lactate biosynthesis gene is from Escherichia coli. In one embodiment, the D-lactate biosynthesis gene and/or the L-lactate biosynthesis gene is from Bacillus coagulans. In one embodiment, the D-lactate biosynthesis gene and/or L-lactate biosynthesis gene is from Clostridium spp. In one embodiment, the Clostridium spp. is Clostridium propionicum. In another embodiment, the D-lactate biosynthesis gene and/or L-lactate biosynthesis gene is from a Megasphaera spp. In one embodiment, the Megasphaera spp. is Megasphaera elsdenii. In another embodiment, the D-lactate biosynthesis gene and/or L-lactate biosynthesis gene is from Prevotella spp. In one embodiment, the Prevotella spp. is Prevotella ruminicola. Other D-lactate biosynthesis genes and/or L-lactate biosynthesis genes are well-known to one of ordinary skill in the art.

In some embodiments, the recombinant bacteria comprise the gene(s) for D-lactate biosynthesis, e.g., ldhA, and/or L-lactate biosynthesis, e.g., ldhL. The gene(s) may be codon-optimized and/or modified, and translational and transcriptional elements may be added.

In one embodiment, the ldhA gene has at least about 80% identity with SEQ ID NO: 3. In another embodiment, the ldhA gene has at least about 85% identity with SEQ ID NO: 3. In one embodiment, the ldhA gene has at least about 90% identity with SEQ ID NO: 3. In one embodiment, the ldhA gene has at least about 95% identity with SEQ ID NO: 3. In another embodiment, the ldhA gene has at least about 96%, 97%, 98%, or 99% identity with SEQ ID NO: 3. Accordingly, in one embodiment, the ldhA gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 3. In another embodiment, the ldhA gene comprises the sequence of SEQ ID NO: 3. In yet another embodiment the ldhA gene consists of the sequence of SEQ ID NO: 3.

In one embodiment, the ldhL gene has at least about 80% identity with SEQ ID NO: 4. In another embodiment, the ldhL gene has at least about 85% identity with SEQ ID NO: 4. In one embodiment, the ldhL gene has at least about 90% identity with SEQ ID NO: 4. In one embodiment, the ldhL gene has at least about 95% identity with SEQ ID NO: 4. In another embodiment, the ldhL gene has at least about 96%, 97%, 98%, or 99% identity with SEQ ID NO: 4. Accordingly, in one embodiment, the ldhL gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 4. In another embodiment, the ldhL gene comprises the sequence of SEQ ID NO: 4. In yet another embodiment the ldhL gene consists of the sequence of SEQ ID NO: 4.

In one embodiment, a polypeptide encoded by the D-lactate biosynthesis gene expressed by the recombinant bacteria has at least about 80% identity with SEQ ID NO: 15. In one embodiment, a polypeptide encoded by the D-lactate biosynthesis gene expressed by the recombinant bacteria has at least about 85% identity with SEQ ID NO: 15. In one embodiment, a polypeptide encoded by the D-lactate biosynthesis gene expressed by the recombinant bacteria has at least about 90% identity with SEQ ID NO: 15. In one embodiment, a polypeptide encoded by the D-lactate biosynthesis gene expressed by the recombinant bacteria has at least about 95% identity with SEQ ID NO: 15. In another embodiment, a polypeptide encoded by the D-lactate biosynthesis gene expressed by the recombinant bacteria has at least about 96%, 97%, 98%, or 99% identity with SEQ ID NO: 15, respectively. Accordingly, in one embodiment, a polypeptide encoded by the D-lactate biosynthesis gene expressed by the recombinant bacteria has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 15. In another embodiment, a polypeptide encoded by the D-lactate biosynthesis gene expressed by the recombinant bacteria comprises the sequence of SEQ ID NO: 15. In another embodiment, a polypeptide encoded by the D-lactate biosynthesis gene expressed by the recombinant bacteria consists of the sequence of SEQ ID NO: 15.

In one embodiment, a polypeptide encoded by the L-lactate biosynthesis gene expressed by the recombinant bacteria has at least about 80% identity with SEQ ID NO: 16. In one embodiment, a polypeptide encoded by the L-lactate biosynthesis gene expressed by the recombinant bacteria has at least about 85% identity with SEQ ID NO: 16. In one embodiment, a polypeptide encoded by the L-lactate biosynthesis gene expressed by the recombinant bacteria has at least about 90% identity with SEQ ID NO: 16. In one embodiment, a polypeptide encoded by the L-lactate biosynthesis gene expressed by the recombinant bacteria has at least about 95% identity with SEQ ID NO: 16. In another embodiment, a polypeptide encoded by the L-lactate biosynthesis gene expressed by the recombinant bacteria has at least about 96%, 97%, 98%, or 99% identity with SEQ ID NO: 16, respectively. Accordingly, in one embodiment, a polypeptide encoded by the L-lactate biosynthesis gene expressed by the recombinant bacteria has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 16. In another embodiment, a polypeptide encoded by the L-lactate biosynthesis gene expressed by the recombinant bacteria comprises the sequence of SEQ ID NO: 16. In another embodiment, a polypeptide encoded by the L-lactate biosynthesis gene expressed by the recombinant bacteria consists of the sequence of SEQ ID NO: 16.

In some embodiments, the D-lactate biosynthesis gene is a synthetic D-lactate biosynthesis gene. In some embodiments, the L-lactate biosynthesis gene is a synthetic L-lactate biosynthesis gene.

In some embodiments, the recombinant bacteria comprise a combination of D-lactate biosynthesis genes and/or L-lactate biosynthesis genes from different species, strains, and/or substrains of bacteria, and are capable of producing D-lactate and L-lactate, respectively. In some embodiments, one or more of the D-lactate biosynthesis genes and/or L-lactate biosynthesis genes is functionally replaced, modified, and/or mutated in order to enhance stability and/or increase D-lactate production and/or L-lactate production. In some embodiments, the recombinant bacteria are capable of expressing the D-lactate biosynthesis cassette and/or L-lactate biosynthesis cassette and producing D-lactate and/or L-lactate, respectively, in low-oxygen conditions, in the presence of certain molecules or metabolites, in the presence of molecules or metabolites associated with liver damage, inflammation or an inflammatory response, or in the presence of some other metabolite that may or may not be present in the gut.

The gene or gene cassette for producing the metabolite, e.g., D-lactate and/or L-lactate, may be present on a plasmid or bacterial chromosome. The gene or gene cassette for producing the metabolite, e.g., D-lactate and/or L-lactate, may be expressed on a high-copy plasmid, a low-copy plasmid, or a chromosome. In some embodiments, expression from the plasmid may be useful for increasing expression of the metabolite, e.g., D-lactate and/or L-lactate. In some embodiments, expression from the chromosome may be useful for increasing stability of expression of the metabolite, e.g., D-lactate and/or L-lactate. In some embodiments, the gene or gene cassette for producing the metabolite, e.g., D-lactate and/or L-lactate, is integrated into the bacterial chromosome at one or more integration sites in the recombinant bacteria. For example, one or more copies of the D-lactate biosynthesis gene cassette and/or L-lactate biosynthesis gene cassette may be integrated into the bacterial chromosome. In some embodiments, the gene or gene cassette for producing the metabolite, e.g., D-lactate and/or L-lactate, is expressed from a plasmid in the recombinant bacteria.

In some embodiments, the bacteria are genetically engineered to include multiple mechanisms of action, e.g., circuits producing multiple copies of the same product (e.g., to enhance copy number) or circuits performing multiple different functions. In some embodiments, the gene or gene cassette for producing the metabolite, e.g., D-lactate and/or L-lactate, is inserted into the bacterial genome at one or more of the following insertion sites in E. coli Nissle: malE/K, araC/BAD, lacZ, thyA, malP/T. For example, the recombinant bacteria may include four copies of the gene, gene(s), or gene cassettes for producing the metabolite, e.g., D-lactate and/or L-lactate, inserted at four different insertion sites. Alternatively, the recombinant bacteria may include three copies of the gene, gene(s), or gene cassettes for producing the metabolite, e.g., D-lactate and/or L-lactate, inserted at three different insertion sites and three copies of the gene, gene(s), or gene cassettes for producing the metabolite, e.g., D-lactate and/or L-lactate, inserted at three different insertion sites. Any suitable insertion site may be used. The insertion site may be anywhere in the genome, e.g., in a gene required for survival and/or growth; in an active area of the genome, such as near the site of genome replication; and/or in between divergent promoters in order to reduce the risk of unintended transcription.

In addition, multiple copies of any gene, gene cassette, or regulatory region may be present in the bacterium, wherein one or more copies of the gene, gene cassette, or regulatory region may be mutated or otherwise altered as described herein. In some embodiments, the recombinant bacteria are engineered to comprise multiple copies of the same gene, gene cassette, or regulatory region in order to enhance copy number or to comprise multiple different components of a gene cassette performing multiple different functions.

In some embodiments, the recombinant bacteria are non-pathogenic bacteria. In some embodiments, the recombinant bacteria are commensal bacteria. In some embodiments, the recombinant bacteria are probiotic bacteria. In some embodiments, non-pathogenic bacteria are Gram-negative bacteria. In some embodiments, non-pathogenic bacteria are Gram-positive bacteria. In some embodiments, the recombinant bacteria are naturally pathogenic bacteria that are modified or mutated to reduce or eliminate pathogenicity. Exemplary bacteria include, but are not limited to Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the recombinant bacteria are selected from the group consisting of Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli Nissle, Lactobacillus acidophilus, Lactobacillus plantarum, Lactobacillus reuteri, and Lactococcus lactis.

In some embodiments, the recombinant bacteria are Escherichia coli strain Nissle 1917 (E. coli Nissle), a Gram-positive bacterium of the Enterobacteriaceae family that “has evolved into one of the best characterized probiotics” (Ukena et al., PLoS One. 2007 Dec. 12; 2(12):e1308). The strain is characterized by its complete harmlessness (Schultz, Inflamm Bowel Dis. 2008 July; 14(7):1012-8), and has GRAS (generally recognized as safe) status (Reister et al., J Biotechnol. 2014 Oct. 10; 187:106-7, emphasis added). Genomic sequencing confirmed that E. coli Nissle lacks prominent virulence factors (e.g., E. coli α-hemolysin, P-fimbrial adhesins) (Schultz, Inflamm Bowel Dis. 2008 July; 14(7):1012-8), In addition, it has been shown that E. coli Nissle does not carry pathogenic adhesion factors, does not produce any enterotoxins or cytotoxins, is not invasive, and is not uropathogenic (Sonnenborn et al., Microbial Ecology in Health and Disease. 2009; 21:122-158). As early as in 1917, E. coli Nissle was packaged into medicinal capsules, called Mutaflor, for therapeutic use. E. coli Nissle has since been used to treat ulcerative colitis in humans in vivo (Rembacken et al., Lancet. 1999 Aug. 21; 354(9179):635-9), to treat inflammatory bowel disease, Crohn's disease, and pouchitis in humans in vivo (Schultz, Inflamm Bowel Dis. 2008 July; 14(7):1012-8), and to inhibit enteroinvasive Salmonella, Legionella, Yersinia, and Shigella in vitro (Altenhoefer et al., FEMS Immunol Med Microbiol. 2004 Apr. 9; 40(3):223-9). It is commonly accepted that E. coli Nissle's “therapeutic efficacy and safety have convincingly been proven” (Ukena et al., PLoS One. 2007 Dec. 12; 2(12):e1308). In a recent study in non-human primates, Nissle was well tolerated by female cynomolgus monkeys after 28 days of daily NG dose administration at doses up to 1×1012 CFU/animal. No Nissle related mortality occurred and no Nissle related effects were identified upon clinical observation, body weight, and clinical pathology assessment (see, e.g., PCT/US16/34200).

One of ordinary skill in the art would appreciate that the genetic modifications disclosed herein may be adapted for other species, strains, and subtypes of bacteria.

Unmodified E. coli Nissle and the recombinant bacteria may be destroyed, e.g., by defense factors in the gut or blood serum (Sonnenborn et al., Microbial Ecology in Health and Disease. 2009; 21:122-158). Thus the recombinant bacteria may require continued administration. Residence time in vivo may be calculated for the recombinant bacteria.

Methods of measuring the level of metabolite, e.g., D-lactate and/or L-lactate, such as, mass spectrometry, gas chromatography, high-performance liquid chromatography (HPLC), are known in the art (see, e.g., Aboulnaga et al., J Bact. 2013; 195(16):3704-3713). In some embodiments, measuring the activity and/or expression of one or more gene products in the D-lactate gene cassette and/or L-lactate gene cassette serves as a proxy measurement for D-lactate production and/or L-lactate production. In some embodiments, the bacterial cells are harvested and lysed to measure D-lactate production and/or L-lactate production. In alternate embodiments, D-lactate production and/or L-lactate production is measured in the bacterial cell medium.

In some embodiments, the recombinant bacterium is capable of producing about 1 mM D-lactate to about 20 mM D-lactate. In some embodiments, the recombinant bacterium is capable of producing about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM or about 20 mM D-lactate. In some embodiments, the recombinant bacterium is capable of producing about 1-20 mM, about 2-20 mM, about 3-20 mM, about 4-20 mM, about 5-20 mM, about 10-20 mM, about 15-20 mM, about 1-15 mM, about 2-15 mM, about 3-15 mM, about 4-15 mM, about 5-10 mM, about 10-15 mM, about 1-10 mM, about 2-10 mM, about 3-10 mM, about 4-10 mM, or about 5-10 mM D-lactate in low-oxygen conditions.

In some embodiments, the recombinant bacterium is capable of producing about 1 μmol/10⁹ cells/hour D-lactate to about 10 μmol/10⁹ cells/hour D-lactate. In some embodiments, the recombinant bacterium is capable of producing about 1 μmol/10⁹ cells/hour, about 2 μmol/10⁹ cells/hour, about 3 μmol/10⁹ cells/hour, about 4 μmol/10⁹ cells/hour, about 5 μmol/10⁹ cells/hour, about 6 μmol/10⁹ cells/hour, about 7 μmol/10⁹ cells/hour, about 8 μmol/10⁹ cells/hour, about 9 μmol/10⁹ cells/hour, or about 10 μmol/10⁹ cells/hour D-lactate. In some embodiments, the recombinant bacterium is capable of producing about 1-10 μmol/10⁹ cells/hour, about 2-10 μmol/10⁹ cells/hour, about 3-10 μmol/10⁹ cells/hour, about 4-10 μmol/10⁹ cells/hour, about 5-10 μmol/10⁹ cells/hour, about 1-5 μmol/10⁹ cells/hour, about 2-5 μmol/10⁹ cells/hour, about 3-5 μmol/10⁹ cells/hour, about 4-5 μmol/10⁹ cells/hour, about 1-2 μmol/10⁹ cells/hour, about 1-3 μmol/10⁹ cells/hour, about 1-4 μmol/10⁹ cells/hour, about 2-3 μmol/10⁹ cells/hour, about 2-4 μmol/10⁹ cells/hour, or about 2-5 μmol/10⁹ cells/hour D-lactate in low-oxygen conditions.

In some embodiments, under conditions where the gene, gene(s), or gene cassettes for producing the metabolite, e.g., D-lactate, is expressed, the recombinant bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the metabolite as compared to unmodified bacteria of the same subtype under the same conditions. Certain unmodified bacteria will not have detectable levels of the metabolite, e.g., D-lactate and/or L-lactate. In embodiments using genetically modified forms of these bacteria, the metabolite, e.g., D-lactate and/or L-lactate, will be detectable under inducing conditions.

In some embodiments, quantitative PCR (qPCR) is used to amplify, detect, and/or quantify mRNA expression levels of the gene, gene(s), or gene cassettes for producing the metabolite, e.g., D-lactate and/or L-lactate. Primers may be designed and used to detect mRNA in a sample according to methods known in the art. In some embodiments, a fluorophore is added to a sample reaction mixture that may contain metabolite RNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the metabolite.

In some embodiments, quantitative PCR (qPCR) is used to amplify, detect, and/or quantify mRNA expression levels of the metabolite. Primers may be designed and used to detect mRNA in a sample according to methods known in the art. In some embodiments, a fluorophore is added to a sample reaction mixture that may contain metabolite mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the metabolite.

### III. Methods

Another aspect of the disclosure provides methods of treating diseases, e.g., autoimmune disease and inflammatory disease, e.g., inflammatory brain disease and multiple sclerosis, by administering to a subject in need thereof, a composition comprising the recombinant bacteria as described herein.

In some embodiments, the autoimmune disease and inflammatory disease is selected from the group consisting of inflammatory brain disease and multiple sclerosis. In some embodiments, the subject to be treated is a human patient.

The method may comprise preparing a pharmaceutical composition with at least one genetically engineered species, strain, or subtype of bacteria described herein, and administering the pharmaceutical composition to a subject in a therapeutically effective amount. In some embodiments, the recombinant bacteria are administered orally, e.g., in a liquid suspension. In some embodiments, the recombinant bacteria are lyophilized in a gel cap and administered orally. In some embodiments, the recombinant bacteria are administered via a feeding tube or gastric shunt. In some embodiments, the recombinant bacteria are administered rectally, e.g., by enema. In some embodiments, the recombinant bacteria are administered topically, intraintestinally, intrajejunally, intraduodenally, intraileally, and/or intracolically.

In certain embodiments, the recombinant bacteria described herein are administered to treat, manage, ameliorate, or prevent autoimmune or inflammatory diseases in a subject. In some embodiments, the method of treating or ameliorating autoimmune or inflammatory diseases allows one or more symptoms of the disease to improve by at least about 5%, 10%, 20%, 30%, 40%, 50%, 60%, 70%, 80%, 90%, 95%, or more as compared to levels in an untreated or control subject. In some embodiments, the symptom (e.g., inflammation, obesity, insulin resistance) is measured by comparing measurements in a subject before and after administration of the recombinant bacteria. In some embodiments, the subject is a human subject.

Before, during, and after the administration of the recombinant bacteria in a subject, metabolites level, metabolic symptoms and manifestations may be measured in a biological sample, e.g., blood, serum, plasma, urine, fecal matter, peritoneal fluid, a sample collected from a tissue, such as liver, skeletal muscle, pancreas, epididymal fat, subcutaneous fat, and beige fat. The biological samples may be analyzed to measure symptoms and manifestations of autoimmune and inflammatory diseases. Useful measurements include measures of lean mass, fat mass, body weight, food intake, GLP-1 levels, endotoxin levels, insulin levels, lipid levels, HbA1c levels, short-chain fatty acid levels, triglyceride levels, and nonesterified fatty acid levels. Useful assays include, but are not limited to, insulin tolerance tests, glucose tolerance tests, pyruvate tolerance tests, assays for intestinal permeability, and assays for glycaemia upon multiple fasting and refeeding time points. In some embodiments, the methods may include administration of the compositions to reduce metabolic symptoms and manifestations to baseline levels, e.g., levels comparable to those of a healthy control, in a subject. In some embodiments, the methods may include administration of the compositions to reduce metabolic symptoms and manifestations to undetectable levels in a subject, or to less than about 1%, 2%, 5%, 10%, 20%, 25%, 30%, 40%, 50%, 60%, 70%, 75%, or 80% of the subject's levels prior to treatment.

In some embodiments, the recombinant bacterium is capable of repressing effector T cells in the subject. In some embodiments, the effector T cells are IFN-γ⁺/CD4 T cells and or IFN-γ⁺/IL-17+/CD4 T cells. In some embodiments, the effector T cells are repressed by at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, and least 2.9-fold, or at least 3.0-fold when compared to a control, wherein the control has not been treated with the recombinant bacterium.

In some embodiments, the recombinant bacterium is capable of increasing expression of HIF-1α in dendritic cells by at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, and least 2.9-fold, or at least 3.0-fold when compared to a control, wherein the control has not been treated with the recombinant bacterium.

In some embodiments, the recombinant bacterium decreases re-stimulation of T cells by at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, and least 2.9-fold, or at least 3.0-fold when compared to a control, wherein the control has not been treated with the recombinant bacterium.

In some embodiments, the recombinant bacterium decreases expression of one or more inflammatory cytokines by at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, and least 2.9-fold, or at least 3.0-fold when compared to a control. In one embodiment, the control has not been treated with the recombinant bacterium. In one embodiment, the one or more inflammatory cytokines are IL-17A, IL-10, and/or IFN-γ.

It has been described that lactate can activate the G-protein coupled receptor, GPR81 (see Ranganathan et al., “GPR81, a Cell-Surface Receptor for Lactate, Regulates Intestinal Homeostasis and Protects Mice from Experimental Colitis,” J Immunol Mar. 1, 2018, 200 (5) 1781-1789, the entire contents of which are expressly incorporated herein by reference). Accordingly, in one embodiment, administration of the bacteria described herein to a subject activates GPR81. In one embodiment, activation of GPR81 in the subject suppresses colonic inflammation and/or regulates immune tolerance in the subject. In one embodiment, activation of GPR81 protects the subject from colitis. In another embodiment, activation of GPR81 treats colitis in a subject. In one embodiment, activation of GPR81 prevents and/or treats colonic inflammation in the subject.

In certain embodiments, the recombinant bacteria are E. coli Nissle. The recombinant bacteria may be destroyed, e.g., by defense factors in the gut or blood serum (Sonnenborn et al., Microbial Ecology in Health and Disease. 2009; 21:122-158) or by activation of a kill switch, several hours or days after administration. Thus, the pharmaceutical composition comprising the recombinant bacteria may be re-administered at a therapeutically effective dose and frequency. In alternate embodiments, the recombinant bacteria are not destroyed within hours or days after administration and may propagate and colonize the gut.

The recombinant bacteria may be administered alone or in combination with one or more additional therapeutic agents, e.g., insulin. An important consideration in the selection of the one or more additional therapeutic agents is that the agent(s) should be compatible with the recombinant bacteria, e.g., the agent(s) must not kill the bacteria. The dosage of the recombinant bacteria and the frequency of administration may be selected based on the severity of the symptoms and the progression of the disorder. The appropriate therapeutically effective dose and/or frequency of administration can be selected by a treating clinician.

In certain embodiments, the recombinant bacteria comprise one or more gene cassettes as described herein, which also modulate the levels of D-lactate and/or L-lactate in a patient, e.g., in the serum and/or in the gut. In certain embodiments, the recombinant bacteria comprise one or more gene cassettes as described herein, which increase D-lactate and/or L-lactate levels in the patient, e.g., in the serum and/or in the gut.

### Treatment In Vivo

The recombinant bacteria may be evaluated in vivo, e.g., in an animal model. Any suitable animal model of an autoimmune or inflammatory disease may be used (see, e.g., Mizoguchi, Prog Mol Biol Transl Sci. 2012; 105:263-320). In some embodiments, the animal is a C57BL/6J mouse that is fed a high fat diet in order to induce obesity and T2DM-related symptoms such as hyperinsulinemia and hyperglycemia. In alternate embodiments, an animal harboring a genetic deficiency that causes an autoimmune or inflammatory disease, e.g., an experimental autoimmune encephalomyelitis (EAE) mouse, is used.

The recombinant bacteria are administered to the mice before, during, or after the onset of obesity and disease. Body weight, food intake, and blood plasma (e.g., triglyceride levels, insulin tolerance tests, glucose tolerance tests, pyruvate tolerance tests) may be assayed to determine the severity and amelioration of disease. Metabolism and physical activity may be measured in metabolic cages. Animals may be sacrificed to assay metabolic tissues such as liver, skeletal muscle, epididymal fat, subcutaneous fat, brown fat, pancreas, and brain, are collected for analysis of histology and gene expression.

The engineered bacteria may be evaluated in vivo, e.g., in an animal model for autoimmune disease, e.g., multiple sclerosis. Any suitable animal model of a disease associated with multiple sclerosis may be used, e.g., experimental autoimmune encephalomyelitis (EAE). Body weight and plasma samples can be taken throughout the duration of the study. Upon conclusion of the study, the mice can be killed, and the liver and intestine can be removed and assayed.

### IV. Pharmaceutical Compositions and Formulations

Pharmaceutical compositions comprising the recombinant bacteria may be used to treat, manage, ameliorate, and/or prevent an autoimmune or inflammatory disease or disorder, e.g., multiple sclerosis. Pharmaceutical compositions comprising one or more recombinant bacteria, alone or in combination with prophylactic agents, therapeutic agents, and/or and pharmaceutically acceptable carriers are provided.

In certain embodiments, the pharmaceutical composition comprises one species, strain, or subtype of bacteria described herein that are engineered to treat, manage, ameliorate, and/or prevent an autoimmune and inflammatory disease or disorder. In alternate embodiments, the pharmaceutical composition comprises two or more species, strains, and/or subtypes of bacteria described herein that are each engineered to treat, manage, ameliorate, and/or prevent an autoimmune and inflammatory disease or disorder.

The pharmaceutical compositions may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The recombinant bacteria may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, immediate-release, pulsatile-release, delayed-release, or sustained release). Suitable dosage amounts for the recombinant bacteria may range from about 10⁵ to 10¹² bacteria, e.g., approximately 10⁵ bacteria, approximately 10⁶ bacteria, approximately 10⁷ bacteria, approximately 10⁸ bacteria, approximately 10⁹ bacteria, approximately 10¹⁰ bacteria, approximately 10¹¹ bacteria, or approximately 10¹¹ bacteria. The composition may be administered once or more daily, weekly, or monthly. The recombinant bacteria may be formulated into pharmaceutical compositions comprising one or more pharmaceutically acceptable carriers, thickeners, diluents, buffers, surface active agents, neutral or cationic lipids, lipid complexes, liposomes, penetration enhancers, carrier compounds, and other pharmaceutically acceptable carriers or agents.

The recombinant bacteria may be administered topically and formulated in the form of an ointment, cream, transdermal patch, lotion, gel, shampoo, spray, aerosol, solution, emulsion, or other form well-known to one of skill in the art. See, e.g., “Remington's Pharmaceutical Sciences,” Mack Publishing Co., Easton, PA. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art.

The recombinant bacteria may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the recombinant bacteria are enterically coated for release into the gut or a particular region of the gut, for example, the small or large intestines. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

In some embodiments, enteric coating materials may be used, in one or more coating layers (e.g., outer, inner and/o intermediate coating layers). Enteric coated polymers remain unionised at low pH, and therefore remain insoluble. But as the pH increases in the gastrointestinal tract, the acidic functional groups are capable of ionisation, and the polymer swells or becomes soluble in the intestinal fluid.

Materials used for enteric coatings include Cellulose acetate phthalate (CAP), Poly(methacrylic acid-co-methyl methacrylate), Cellulose acetate trimellitate (CAT), Poly(vinyl acetate phthalate) (PVAP) and Hydroxypropyl methylcellulose phthalate (HPMCP), fatty acids, waxes, Shellac (esters of aleurtic acid), plastics and plant fibers. Additionally, Zein, Aqua-Zein (an aqueous zein formulation containing no alcohol), amylose starch and starch derivatives, and dextrins (e.g., maltodextrin) are also used. Other known enteric coatings include ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate.

Coating polymers also may comprise one or more of, phthalate derivatives, CAT, HPMCAS, polyacrylic acid derivatives, copolymers comprising acrylic acid and at least one acrylic acid ester, Eudragit™ S (poly(methacrylic acid, methyl methacrylate)1:2); Eudragit L100™ S (poly(methacrylic acid, methyl methacrylate)1:1); Eudragit L30D™, (poly(methacrylic acid, ethyl acrylate)1:1); and (Eudragit L100-55) (poly(methacrylic acid, ethyl acrylate)1:1) (Eudragit™ L is an anionic polymer synthesized from methacrylic acid and methacrylic acid methyl ester), polymethyl methacrylate blended with acrylic acid and acrylic ester copolymers, alginic acid, ammonia alginate, sodium, potassium, magnesium or calcium alginate, vinyl acetate copolymers, polyvinyl acetate 30D (30% dispersion in water), a neutral methacrylic ester comprising poly(dimethylaminoethylacrylate) (“Eudragit E™), a copolymer of methylmethacrylate and ethylacrylate with trimethylammonioethyl methacrylate chloride, a copolymer of methylmethacrylate and ethylacrylate, Zein, shellac, gums, or polysaccharides, or a combination thereof.

Coating layers may also include polymers which contain Hydroxypropylmethylcellulose (HPMC), Hydroxypropylethylcellulose (HPEC), Hydroxypropylcellulose (HPC), hydroxypropylethylcellulose (HPEC), hydroxymethylpropylcellulose (HMPC), ethylhydroxyethylcellulose (EHEC) (Ethulose), hydroxyethylmethylcellulose (HEMC), hydroxymethylethylcellulose (HMEC), propylhydroxyethylcellulose (PHEC), methylhydroxyethylcellulose (M H EC), hydrophobically modified hydroxyethylcellulose (NEXTON), carboxymethyl hydroxyethylcellulose (CMHEC), Methylcellulose, Ethylcellulose, water soluble vinyl acetate copolymers, gums, polysaccharides such as alginic acid and alginates such as ammonia alginate, sodium alginate, potassium alginate, acid phthalate of carbohydrates, amylose acetate phthalate, cellulose acetate phthalate (CAP), cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS).

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the recombinant bacteria.

In certain embodiments, the recombinant bacteria may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The recombinant bacteria may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The recombinant bacteria may be administered and formulated as depot preparations. Such long acting formulations may be administered by implantation or by injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, the disclosure provides pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

Dosage regimens may be adjusted to provide a therapeutic response. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The recombinant bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

Dosing can depend on several factors, including severity and responsiveness of the disease, route of administration, time course of treatment (days to months to years), and time to amelioration of the disease. Toxicity and therapeutic efficacy of compounds provided herein can be determined by standard pharmaceutical procedures in cell culture or animal models. For example, LD₅₀, ED₅₀, EC₅₀, and IC₅₀ may be determined, and the dose ratio between toxic and therapeutic effects (LD₅₀/ED₅₀) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

### V. Kits

In certain aspects, the instant disclosure provides kits that include a pharmaceutical formulation including a recombinant bacterium for production of D-lactate and/or L-lactate, and a package insert with instructions to perform any of the methods described herein.

In some embodiments, the kits include instructions for using the recombinant bacterium to treat an autoimmune and inflammatory disease or disorder, e.g. multiple sclerosis. The instructions will generally include information about the use of the recombinant bacterium to treat an autoimmune and inflammatory disease or disorder, e.g. multiple sclerosis. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters.

In some embodiments, the kit includes a pharmaceutical formulation including a recombinant bacterium for production of D-lactate and/or L-lactate, an additional therapeutic agent, and a package insert with instructions to perform any of the methods described herein.

The kit may be packaged in a number of different configurations such as one or more containers in a single box. The different components can be combined, e.g., according to instructions provided with the kit. The components can be combined according to a method described herein, e.g., to prepare and administer a pharmaceutical composition.

In some embodiments, the kit can comprise one or more containers with appropriate positive and negative controls or control samples, to be used as standard(s) for detection, calibration, or normalization.

The kit can further comprise a second container comprising a pharmaceutically-acceptable buffer, such as (sterile) phosphate-buffered saline, Ringer's solution, or dextrose solution; and other suitable additives such as penetration enhancers, carrier compounds and other pharmaceutically acceptable carriers or excipients, as described herein. It can further include other materials desirable from a commercial and user standpoint, including other buffers, diluents, filters, and package inserts with instructions for use. The kit can also include a drug delivery system such as liposomes, micelles, nanoparticles, and microspheres, as described herein. The kit can further include a delivery device, such as needles, syringes, pumps, and package inserts with instructions for use.

This invention is further illustrated by the following examples which should not be construed as limiting. The entire contents of all references, patents and published patent applications cited throughout this application, as well as the Figures and the Sequence Listing, are hereby incorporated herein by reference.

### EXAMPLES

The present disclosure is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Example 1. Generation of Various Recombinant Bacterial Strains

Table 6 lists all the bacterial strains used herein. Escherichia coli Nissle 1917 (EcN), designated as SYN001 here, was purchased from the German Collection of Microorganisms and Cell Cultures (DSMZ Braunschweig, E. coli DSM 6601). ldhA gene was codon optimized for E. coli expression and synthesized by IDTDNA. The fragment was then inserted into the vector with origin of replication pSC101, carbicillin resistance and either temperature sensitive promoter PcI857 or anaerobic inducible promoter PfnrS resulting in plasmid logic 1919 and logic 1920 (sequences in Table 1). The plasmids were then transformed into strain SYN6527 where the pta gene was knocked out using lambda red recombination technique to better push carbon flux though lactate production. The plasmids were also transformed into strain SYN6524 and SYN6265 where the adhE and pfkA genes, respectively, were knocked out.

TABLE 6
Strains

| | | | Induction |
|---|---|---|---|
| Strain # | Genotype | Description | Activity |
| SYN001 | Control bacterium | N/A | N/A |
| SYN094 | SYN001 with strep | Control bacterium with strep resistance | N/A |
| | resistance | | |
| SYN6524 | SYN001, ΔadhE | Bacterium with deleted adhE gene | N/A |
| SYN6525 | SYN001, ΔadhE, | Bacterium with plasmid containing | 37° C. |
| | pSC101-cI857-ldhA- | ldhA gene under the control of | |
| | carb | temperature sensitive promoter | |
| SYN6526 | SYN001, ΔadhE, | Bacterium with plasmid containing | Hypoxia |
| | pSC101-fnr-ldhA- | ldhA gene under the control of PfnrS | |
| | carb | inducible promoter | |
| SYN6527 | SYN001, Δpta | Bacterium with deleted pta gene | N/A |
| SYN6528 | SYN001, Δpta, | Bacterium with plasmid containing | 37° C. |
| | pSC101-cI857-ldhA- | ldhA gene under the control of | |
| | carb | temperature sensitive promoter | |
| SYN6529 | SYN001, Δpta, | Bacterium with plasmid containing | Hypoxia |
| | pSC101-fnr-ldhA- | ldhA gene under the control of PfnrS | |
| | carb | inducible promoter | |
| SYN6265 | SYN001, ΔpfkA-Kan | Bacterium with deleted pfkA gene | N/A |
| SYN6530 | SYN001, ΔpfkA-Kan, | Bacterium with plasmid containing | 37° C. |
| | PSC101-cI857-ldhA- | ldhA gene under the control of | |
| | carb | temperature sensitive promoter | |
| SYN6531 | SYN001, ΔpfkA-Kan, | Bacterium with plasmid containing | Hypoxia |
| | pSC101-fnr-ldhA- | ldhA gene under the control of PfnrS | |
| | carb | inducible promoter | |
| SYN6522 | SYN001, STRP, | Bacterium with plasmid containing | 37° C. |
| | PSC101-cI857-ldhA- | ldhA gene under the control of | |
| | carb | temperature sensitive promoter | |
| SYN6523 | SYN001, STRP, | Bacterium with plasmid containing | Hypoxia |
| | pSC101-fnr-ldhA- | ldhA gene under the control of PfnrS | |
| | carb | inducible promoter | |
| SYN6564 | SYN001, ΔadhE-kan, | Bacterium with deleted pta and adhE | N/A |
| | Δpta | genes | |
| SYN6593 | SYN001, ΔadhE-kan, | Bacterium with deleted pta and adhE | 37° C. |
| | Δpta, pSC101-cI857- | genes and with plasmid containing | |
| | ldhA-carb | ldhA gene under the control of | |
| | | temperature sensitive promoter | |
| SYN6594 | SYN001, ΔadhE-kan, | Bacterium with deleted pta and adhE | Hypoxia |
| | Δpta, pSC101-fnr- | genes and with plasmid containing | |
| | ldhA-carb | ldhA gene under the control of PfnrS | |
| | | inducible promoter | |
| SYN6509 | ΔldhA, ΔadhE, | Bacterium with deleted ldhA, mgsA, | N/A |
| | ΔmgsA, ΔfrdBC, | frdBC, pflB, ackA and adhE genes | |
| | ΔpflB::CamR, | | |
| | ΔackA::KanR | | |
| SYN6580 | ΔldhA, ΔadhE, | Bacterium with deleted ldhA, mgsA, | 37° C. |
| | ΔmgsA, ΔfrdBC, | frdBC, pflB, ackA and adhE genes and | |
| | ΔpflB::CamR, | plasmid containing ldhA gene under the | |
| | ΔackA::KanR, | control of cI857 promoter | |
| | pSC101-cI857-ldhA- | | |
| | carb | | |
| SYN6581 | ΔldhA, ΔadhE, | Bacterium with deleted ldhA, mgsA, | Hypoxia |
| | ΔmgsA, ΔfrdBC, | frdBC, pflB, ackA and adhE genes and | |
| | ΔpflB::CamR, | plasmid containing ldhA gene under the | |
| | ΔackA::KanR, | control of fnr promoter | |
| | pSC101-fnr-ldhA- | | |
| | carb | | |

OD₆₀₀ of 1.0 was assumed to be equal to 10⁹ cells/mL in this method. A volume was calculated to target 1 mL of 2×10⁹ cells/mL cell resuspension, and the cells were transferred into a 96-deep well plate and washed once with cold PBS. After centrifugation (4000 rpm, 4° C., 10 min), the PBS was discarded, and the cell pellets were then resuspended in 1 mL of 1×M9+50 mM MOPS +0.5% glucose (MMG) buffer. Eight hundred (800) μL of each sample was transferred into a new 96-deep well plate and 800 μL of MMG, mixed well by pipetting. The plate was then covered by a breathable membrane and moved to an anaerobic chamber to incubate at 37° C. Samples were collected at 5 hours after incubation in the anaerobic chamber. The samples were centrifuged for 10 minutes at 4000 rpm at 4° C. immediately after collection. A sample of 100 μL of the supernatant was transferred into a new 96-well plate and stored at −80° C. for future analysis. For D-lactate analysis, the kit purchased from Abcam was used for quantification.

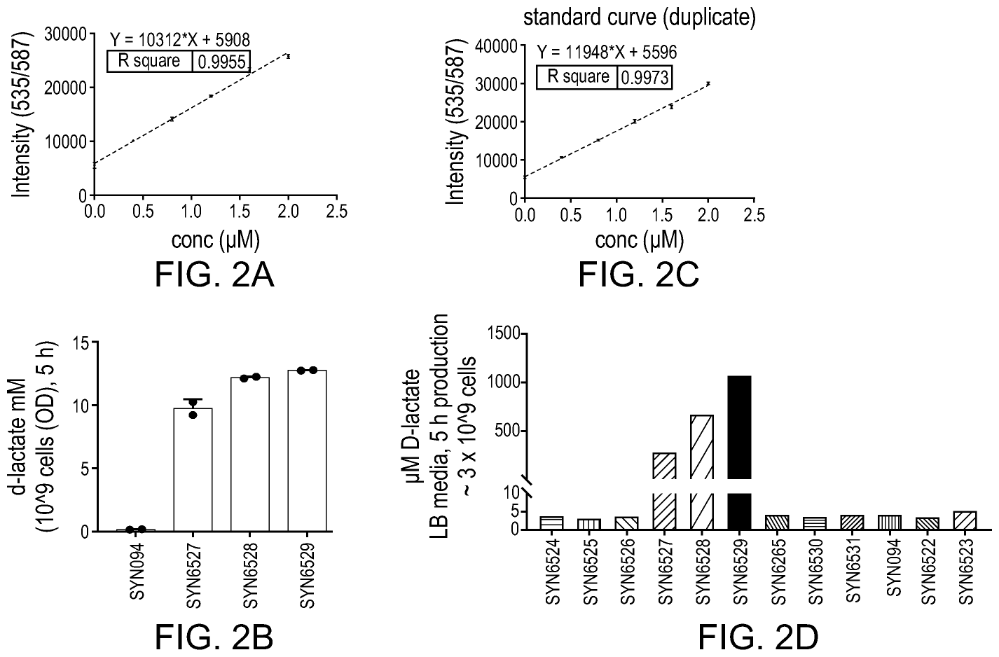

Results are depicted in FIGS. 2A-2D, as well as Table 7, below. Surprisingly, LdhA expressed with Δpta resulted in increased D-lactate production in both SYN6528 and SYN6529 harboring ldhA expression plasmids under control of temperature sensitive promoter pcI857 and anaerobic inducible promoter PfnrS, respectively, when compared to strains with only Δpta (SYN6527) or the wild-type control (SYN094).

TABLE 7
Results from FIGS. 2A and 2B

| | Average CFU/mL | STD |
|---|---|---|
| Strain | (10\hat{ }10) | (10\hat{ }10) |
| SYN094 | 5.87 | 0.51 |
| SYN6527 | 14.33 | 1.15 |
| SYN6528 | 22.33 | 2.08 |
| SYN6529 | 23.00 | 2.65 |

### Example 2. L-lactate Recombinant Bacterial Strains

Strains are constructed as described in Example 1. Table 8 lists of the strains that will be used for L-lactate production.

TABLE 8
L-lactate strains

| Strain | | | Induction |
|---|---|---|---|
| number | Genotype | Description | Activity |
| SYN001 | Control bacterium | N/A | N/A |
| V0 | SYN001, ΔadhE, Δpta, | Bacterium with deleted adhE, pta, | Hypoxia |
| | ΔldhA::Pfnr-ldhLBcoagulans | ldhA genes; ldhL gene under the | |
| | | control of PfnrS promoter | |
| V1 | SYN001, ΔadhE, Δpta, | Bacterium with deleted adhE, pta, | 37° C. |
| | ΔldhA::PcI857-ldhLBcoagulans | ldhA genes; ldhL gene under the | |
| | | control of temperature sensitive | |
| | | promoter | |
| V2 | SYN001, ΔadhE, Δpta, ΔpflB, | Bacterium with deleted adhE, pta, | N/A |
| | ΔfrdA, (ΔackA) | pflB, frdA, ackA, and ldhA genes | |
| V3 | SYN001, ΔadhE, Δpta, ΔpflB, | Bacterium with deleted adhE, pta, | N/A |
| | ΔfrdA, (ΔackA), ΔldhA::PldhA- | pflB, frdA, ackA, and ldhA genes; | |
| | ldhLBcoagulans | ldhL gene under the control of | |
| | | PldhA promoter | |
| V4 | SYN001, ΔadhE, Δpta, ΔpflB, | Bacterium with deleted adhE, pta, | Hypoxia |
| | ΔfrdA, (ΔackA), ΔldhA::Pfnr- | pflB, frdA, ackA, and ldhA genes; | |
| | ldhLBcoagulans | ldhL gene under the control of | |
| | | PfnrS promoter | |
| V5 | SYN001, ΔadhE, Δpta, ΔpflB, | Bacterium with deleted adhE, pta, | 37° C. |
| | ΔfrdA, (ΔackA),ΔldhA::PcI857- | pflB, frdA, ackA, and ldhA genes; | |
| | ldhLBcoagulans | ldhL gene under the control of | |
| | | temperature sensitive promoter | |
| V6 | SYN001, ΔadhE, Δpta, ΔpflB, | Bacterium with deleted adhE, pta, | Hypoxia |
| | ΔfrdA, (ΔackA), ΔldhA::PfnrA- | pflB, frdA, ackA, ldhA, poxB, pps, | |
| | ldhLBcoagulans, ΔpoxB, Δpps, | dld, and lldD genes; ldhL gene | |
| | Δdld, ΔlldD | under the control of PfnrS promoter | |

### Example 3. L-Lactate Production by Recombinant Bacterial Strains

EAE was induced in 8-10 week old female C56BL6/J mice by subcutaneous immunization with 150 mg MOG35-55 peptide, MEVGWYRPPFSRVVHLYRNGK (SEQ ID NO: 29) (Genemed Synthesis) emulsified in 200 mL of complete Freund's adjuvant (Invivogen) per mouse, followed by administration of 100 mL PBS containing 200 ng pertussis toxin (List biological Laboratories) on days 0 and 2. Mice were monitored and scored daily thereafter. Clinical signs of EAE were assessed as follows: 0, no signs of disease; 1, loss of tone in the tail; 2, hind limb paresis; 3, hind limb paralysis; 4, tetraplegia; 5, moribund.

For testing the effects of bacteria on EAE, mice were orally administrated the control bacteria (SYN094) or engineered bacteria producing D-Lactate (SYN6528, D-Lactate production under temperature promoter). Daily bacterial administrations at the dose of ˜1e10 CFUs per mouse started on day −3 and continued throughout the experiment.

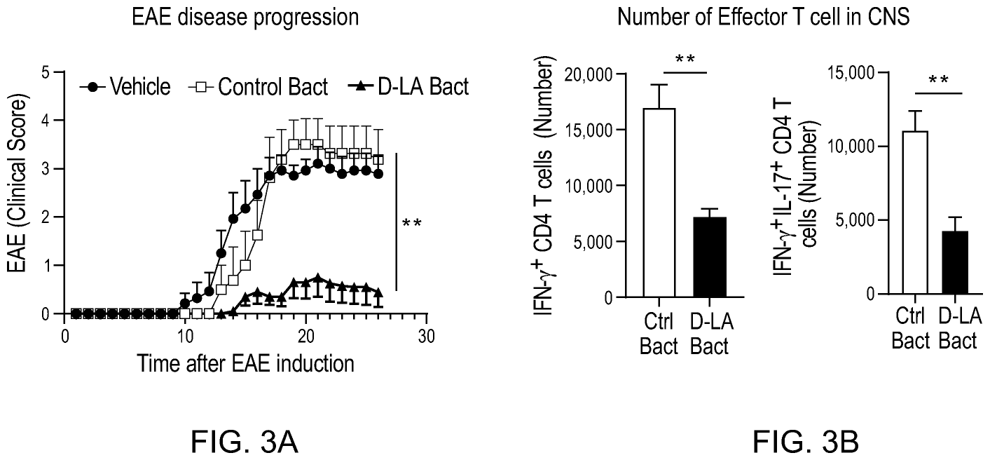

The engineered bacterial strain producing D-Lactate in the mouse gut suppressed neuroinflammation, ameliorates development of experimental autoimmune encephalomyelitis (EAE) (FIG. 3A). Disease progression of EAE was decreased SYN6528. EAE mice that received SYN6528 remained at no signs of disease or loss of tone in the tail after approximately 18 days after induction of EAE (15 days after beginning daily administration of bacteria). In comparison, disease in EAE mice treated with SYN094 or vehicle only controls experienced disease progression of hind limb paralysis or tetraplegia after approximately 18 days after induction of EAE (15 days after beginning daily administration of bacteria).

To evaluate the amount of effector T cells in the mouse brain, mononuclear cells were isolated from the CNS. Briefly, mice were perfused with 1×PBS and the isolated brain was homogenized with a razor blade, digested in 0.66 mg/mL Papain (Sigma-Aldrich)-contained HBSS solution for 15 min at 37° C. and then incubated another 15 min after equal volume of DMEM medium supplied with Collagenase D (Roche) and DNase I (Thermo Fisher Scientific) in the concentration of 0.66 mg/mL and 8 U/mL respectively is added. The digested CNS homogenize was filtered through a 70 mm cell strainer and centrifuged at 1400 rpm at 4° C. for 5 min followed by suspension of the pellet in 30% Percoll™ (GE Healthcare) in 1×PBS. The suspension was centrifuged at 1600 rpm at room temperature for 24 min with slow acceleration and deceleration settings for separation of myelin and cells. Single CNS cell suspensions were stimulated with 50 ng/mL phorbol 12-myristate 13-acetate (PMA, Sigma-Aldrich, #P8139), 1 μM Ionomycin (Sigma-Aldrich, #I3909-1ML), GolgiStop (BD Biosciences, #554724, 1:1500) and GolgiPlug (BD Biosciences, #555029, 1:1500) diluted in RPMI (Life Technologies, #11875119) containing 10% FBS, 1% penicillin/streptomycin, 50 μM 2-metcaptoethanol (Sigma-Aldrich, #M6250), and 1% non-essential amino acids (Life Technologies, #11140050). After 4 hours, cell suspensions were washed with 0.5% BSA, 2 mM EDTA in 1×PBS and incubated with surface antibodies and a live/dead cell marker on ice. After 30 min, cells were washed with 0.5% BSA, 2 mM EDTA in 1×PBS and fixed according to the manufacturer's protocol of an intracellular labeling kit (eBiosciences, #00-5523-00). Surface antibodies used in this study were: BUV661 anti-mouse CD45 (BioLegend, #103147, 1:100); PeCy7 anti-mouse CD4 (BioLegend, #100422, 1:100); BV750 anti-mouse CD3 (BioLegend, #100249, 1:100). Intracellular antibodies were: APC/Cy7 anti-mouse IFN-γ (BD Biosciences, #561479, 1:100); PE anti-mouse IL-17A (BioLegend, #506904, 1:100). Cells were acquired on a Symphony A5 (BD Biosciences) and analyzed on Flowjo 10 (Becton Dickinson).

The engineered bacterial strain producing D-Lactate in the mouse gut decreased the number of pathogenic effector T cells in the mouse brain (FIG. 3B). SYN6528 decreased the number of IFN-γ⁺/CD4 T cells and IFN-γ⁺/IL-17⁺/CD4 T cells by approximately by 2-fold of when compared to SYN094 and vehicle only controls.

To analyze DCs by flow cytometry, splenic cell suspensions were incubated with surface antibodies and a live/dead cell marker on ice. After 30 min, cells were washed with 0.5% BSA, 2 mM EDTA in 1×PBS and fixed according to the manufacturer's protocol (eBiosciences, #00-5523-00). Intracellular staining was performed for 1 h at room temperature. Surface antibodies used in this study were: BUV395 anti-mouse MHC-II (Invitrogen, #17-5321-82, 1:200); BUV496 anti-mouse CD24 (BD Biosciences, #564664, 1:100); BUV563 anti-mouse Ly-6G (BD Biosciences, #612921, 1:100); BUV661 anti-mouse CD45 (BioLegend, #103147, 1:100); BV570 anti-mouse Ly-6C (BioLegend, #128030, 1:100); BV605 anti-mouse CD80 (BD Biosciences, #563052, 1:100); BV786 anti-mouse CD11b (BioLegend, #101243, 1:100); PE-Texas Red anti-mouse CD11c (BioLegend, #117348, 1:100); APC anti-mouse/human CD45R/B220 (BioLegend, #103212, 1:100); APC-R700 anti-mouse CD103 (BD Biosciences, #565529, 1:100); APC/Cy7 anti-mouse F4/80 (BioLegend, #123118, 1:100). Intracellular antibody used was Alexa Fluor 488 anti-mouse HIF-1α (Bioss Antibodies, #BS-0737R-A488, 1:100). FACs was performed on a Symphony A5 (BD Biosciences).

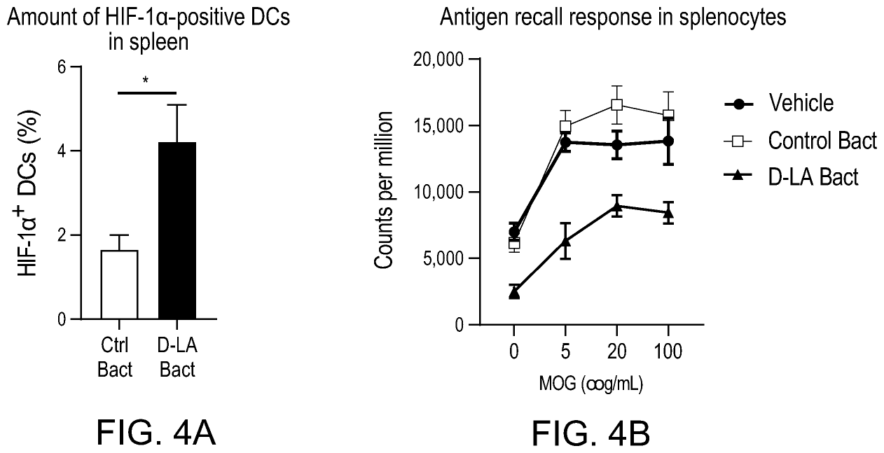

D-Lactate-producing bacteria ameliorates EAE through increased HIF-1α expression in dendritic cells (DCs) leading to immunoregulation and control of T cell compartment. Increased percentage of anti-inflammatory HIF-1α-positive DCs after treatment with SYN6528 (FIG. 4A). HIF-1α-positive DCs increased after treatment with SYN6528 by approximately by 2-fold.

For recall proliferative responses to MOG peptide (EAE antigen), splenocytes were cultured in complete RPMI medium for 72 h at a density of 4×10e5 cell/well in 96 well plates in the presence of MOG35-55 peptide (Genemed Synthesis). During the final 16 h, cells are pulsed with 1 μCi [3H]thymidine (PerkinElmer) followed by collection on glass fiber filters (PerkinElmer) and analysis of incorporated [3H]thymidine in a beta-counter (1450 MicroBeta TriLux; PerkinElmer). The concentrations of MOG peptide were: 0, 5, 20, 100 ug/ml.

Lower recall response to MOG35-55 (EAE antigen) re-stimulation in splenocytes (T cells) from SYN6528 treated mice (FIG. 4B). Splenocytes from mice treated with vehicle, SYN094, or SYN6528, proliferated in a dose depended manner when exposed to MOG35-55. Cells from SYN6528 mice did not proliferate at least by 1.5-fold in comparison to the vehicle and SYN094 controls.

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

We claim:

1. A recombinant bacterium comprising an ldhA gene for producing D-lactate, wherein the ldhA gene is operably linked to a directly or indirectly inducible promoter that is not associated with the ldhA gene in nature and is induced by exogenous environmental conditions.

2. The bacterium of claim 1, wherein the bacterium comprises a deletion or mutation in one or more genes selected from the group comprising of phosphate acetyltransferase (pta), formate acetyltransferase 1 (pflB), and/or acetate kinase (ackA).

3. The bacterium of claim 2, wherein the bacterium comprises a deletion or mutation in the pta gene.

4. The bacterium of claim 2 or claim 3, wherein the bacterium comprises a deletion or mutation in the ackA gene.

5. The bacterium of any one of claims 2-4, wherein the bacterium comprises a deletion or mutation in the pflB gene.

6. The bacterium of any one of claims 1-5, further comprising a ribosome binding site before the ldhA gene.

7. The bacterium of any one of claims 1-6, wherein the promoter is directly or indirectly induced by low-oxygen or anaerobic conditions.

8. The bacterium of claim 7, wherein the promoter is an FNR-inducible promoter.

9. The bacterium of any one of claims 1-6, wherein the promoter is induced by temperature.

10. The bacterium of claim 9, wherein the promoter is a cI857 promoter.

11. The bacterium of any one of the previous claims, wherein the ldhA gene is present on a plasmid in the bacterium.

12. The bacterium of any one of claims 1-10, wherein the ldhA gene is present on a chromosome in the bacterium.

13. The bacterium of any one of the previous claims, wherein the bacterium is a non-pathogenic bacterium.

14. The bacterium of any one of the previous claims, wherein the bacterium is a probiotic or a commensal bacterium.

15. The bacterium of any one of the previous claims, wherein the bacterium is selected from the group consisting of Bacteroides, Bifidobacterium, Clostridium, Escherichia, Lactobacillus, and Lactococcus.

16. The bacterium of claim 15, wherein the bacterium is Escherichia coli strain Nissle.

17. The bacterium of any one of the previous claims, wherein the bacterium is capable of producing about 1 mM D-lactate to about 20 mM D-lactate in vitro.

18. The bacterium of any of the previous claims, wherein the bacterium is capable of producing about 1 μmol/10⁹ cells/hour, 2 μmol/10⁹ cells/hour, or 3 μmol/10⁹ cells/hour D-lactate in vitro.

19. The bacterium of claim 18, wherein the bacterium us capable of producing 2 μmol/10⁹ cells/hour D-lactate in vitro.

20. A pharmaceutically acceptable composition comprising the bacterium of any one of the previous claims; and a pharmaceutically acceptable carrier.

21. The pharmaceutically acceptable composition of claim 20, wherein the composition is formulated for oral administration.

22. A method of treating a disease or disorder in a subject in need thereof comprising the step of administering to the subject the pharmaceutical composition of claim 20 or claim 21, thereby treating the disease or disorder.

23. The method of claim 22, wherein the disease or disorder is an autoimmune disease or inflammatory disease or disorder.

24. The method of claim 22, wherein the disease or disorder selected from the group consisting of multiple sclerosis, central nervous system inflammation (CNS) inflammation, 2,4,6-trinitrobenzene sulfonic acid (TNBS)-induced colitis, T cell-induced colitis, T cell-induced small bowel inflammation, chronic colitis, rheumatoid arthritis, celiac disease, myasthenia gravis, and B-cell-mediated T-cell-dependent autoimmune disease.

25. A method of treating, reducing, or ameliorating symptoms of a disease or disorder in a subject in need thereof comprising the step of administering to the subject the pharmaceutical composition of claim 20 or claim 21, wherein the symptom of the disease or disorder is inflammation.

26. The method of any one of claims 22-25, wherein the subject has an increased level of D-lactate after the composition is administrated.

27. The method of any one of claims 22-26, wherein the subject is a human.

28. The method of any one of claims 22-27, wherein the method further comprises
(a) measuring a level of D-lactate in urine of the subject at a first time point prior to administration of the pharmaceutical composition;
(b) measuring a level of D-lactate in urine of the subject at a second time point after administration of the pharmaceutical composition; wherein an increase in the level of D-lactate in the urine of the subject at the second time point as compared to the first time point indicates that the treatment is efficacious.

29. The method of any one of claims 22-28, wherein administration of the pharmaceutical composition represses effector T cells by at least 1.5 fold, at least 1.8-fold, at least 2-fold, at least 2.2-fold, or at least 2.5-fold when compared to a control, wherein the control has not been treated with the pharmaceutical composition.

30. The method of claim 29, wherein the effector T cells are repressed by at least 2-fold when compared to the control.

31. The method of claim 29 or claim 30, wherein the effector T cells are IFN-γ⁺/CD4 T cells and/or IFN-γ⁺/IL-17⁺/CD4 T cells.

32. The method of any one of claims 22-31, wherein administration of the pharmaceutical composition increases expression of Hypoxia-inducible factor 1-alpha (HIF-1α) in dendritic cells by at least 1.5 fold, at least 1.8-fold, at least 2-fold, at least 2.2-fold, at least 2.5-fold, or at least 3-fold when compared to a control, wherein the control has not been treated with the pharmaceutical composition.

33. The method of claim 32, wherein the expression of HIF-1α is increased by at least 2-fold when compared to the control.

34. The method of any one of claims 22-33, wherein administration of the pharmaceutical composition decreases re-stimulation of T cells by at least 1.5 fold, at least 1.8-fold, at least 2-fold, at least 2.2-fold, or at least 2.5-fold when compared to a control, wherein the control has not been treated with the pharmaceutical composition.

35. A recombinant bacterium comprising an ldhL gene for producing L-lactate, wherein the ldhL gene is operably linked to a directly or indirectly inducible promoter that is not associated with the ldhL gene in nature and is induced by exogenous environmental conditions.

36. The bacterium of claim 35, wherein the bacterium further comprises a deletion or mutation in one or more genes selected from the group comprising of phosphate acetyltransferase (pta), formate acetyltransferase 1 (pflB), and/or acetate kinase (ackA).

37. The bacterium of claim 36, wherein the bacterium comprises a deletion or mutation in the pta gene.

38. The bacterium of claim 36 or claim 37, wherein the bacterium comprises a deletion or mutation in the ackA gene.

39. The bacterium of any one of claims 36-38, wherein the bacterium comprises a deletion or mutation in the pflB gene.

40. The bacterium of any one of claims 35-38, further comprising a ribosome binding site before the ldhL gene.

41. The bacterium of any one of claims 35-38, wherein the promoter is directly or indirectly induced by low-oxygen or anaerobic conditions.

42. The bacterium of claim 41, wherein the promoter is an FNR-inducible promoter.

43. The bacterium of any one of claims 35-40, wherein the promoter is induced by temperature.

44. The bacterium of claim 43, wherein the promoter is a cI857 promoter.

45. The bacterium of any one of claims 35-44, wherein the ldhL gene is present on a plasmid in the bacterium.

46. The bacterium of any one of claims 35-44, wherein the ldhL gene is present on a chromosome in the bacterium.

47. The bacterium of any one of claims 35-46, wherein the bacterium is a non-pathogenic bacterium.

48. The bacterium of any one of claims 35-47, wherein the bacterium is a probiotic or a commensal bacterium.

49. The bacterium of any one of claims 35-48, wherein the bacterium is selected from the group consisting of Bacteroides, Bifidobacterium, Clostridium, Escherichia, Lactobacillus, and Lactococcus.

50. The bacterium of claim 49, wherein the bacterium is Escherichia coli strain Nissle.

51. A pharmaceutically acceptable composition comprising the bacterium of any one of claims 35-50; and a pharmaceutically acceptable carrier.

52. The pharmaceutically acceptable composition of claim 51, wherein the composition is formulated for oral administration.

53. A method of treating a disease or disorder in a subject in need thereof comprising the step of administering to the subject the pharmaceutical composition of claim 51 or claim 52, thereby treating the disease or disorder.

54. The method of claim 53, wherein the disease or disorder is an autoimmune disease or inflammatory disease or disorder.

55. The method of claim 54, wherein the disease or disorder selected from the group consisting of multiple sclerosis, central nervous system inflammation (CNS) inflammation, 2,4,6-trinitrobenzene sulfonic acid (TNBS)-induced colitis, T cell-induced colitis, T cell-induced small bowel inflammation, chronic colitis, rheumatoid arthritis, celiac disease, myasthenia gravis, and B-cell-mediated T-cell-dependent autoimmune disease.

56. A method of treating, reducing, or ameliorating symptoms of a disease or disorder in a subject in need thereof comprising the step of administering to the subject the pharmaceutical composition of claim 51 or claim 52, wherein the symptom of the disease or disorder is inflammation.

57. The method of any one of claims 53-56, wherein the subject has an increased level of L-lactate after the composition is administrated.

58. The method of any one of claims 53-57, wherein the subject is a human.

59. The method of any one of claims 53-58, wherein the method further comprises
(a) measuring a level of L-lactate of the subject at a first time point prior to administration of the pharmaceutical composition;
(b) measuring a level of L-lactate of the subject at a second time point after administration of the pharmaceutical composition; wherein an increase in the level of L-lactate in the urine of the subject at the second time point as compared to the first time point indicates that the treatment is efficacious.

60. The method of any one of claims 53-59, wherein administration of the pharmaceutical composition represses effector T cells by at least 1.5 fold, at least 1.8-fold, at least 2-fold, at least 2.2-fold, or at least 2.5-fold when compared to a control, wherein the control has not been treated with the pharmaceutical composition.

61. The method of claim 60, wherein the effector T cells are repressed by at least 2-fold when compared to the control.

62. The method of claim 60 or claim 61, wherein the effector T cells are IFN-γ⁺/CD4 T cells and/or IFN-γ⁺/IL-17⁺/CD4 T cells.

63. The method of any one of claims 53-62, wherein administration of the pharmaceutical composition increases expression of Hypoxia-inducible factor 1-alpha (HIF-1α) in dendritic cells by at least 1.5 fold, at least 1.8-fold, at least 2-fold, at least 2.2-fold, at least 2.5-fold, or at least 3-fold when compared to a control, wherein the control has not been treated with the pharmaceutical composition.

64. The method of claim 63, wherein the expression of HIF-1α is increased by at least 2-fold when compared to the control.

65. The method of any one of claims 53-64, wherein administration of the pharmaceutical composition decreases re-stimulation of T cells by at least 1.5 fold, at least 1.8-fold, at least 2-fold, at least 2.2-fold, or at least 2.5-fold when compared to a control, wherein the control has not been treated with the pharmaceutical composition.


SEQUENCE LISTING (35 sequences)

SEQ ID NO: 1 - DNA, 6150 bp, Artificial Sequence
  Description of Artificial Sequence Synthetic polynucleotide
aaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcgctcccggagacggtcacagcttgtcaaaaaaaaaccccgcttcggcggggtttttttttggtacctcatcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcataagtgaggatccaaagtgaactctagaaataattttgtttaactttaagaaggaggtatacatatgaaacttgctgtatatagtaccaaacagtacgacaaaaagtaccttcaacaggtcaacgagagctttggtttcgaacttgaatttttcgactttttacttaccgagaaaacggcaaaaacggcgaacggatgtgaagcggtttgcattttcgtcaacgacgacggcagccgccctgttttagaagagttaaagaaacatggagttaaatacatcgcattacgttgtgcaggtttcaacaacgttgatctggatgctgcgaaggaactgggattgaaagttgtgcgcgtgcccgcttatgacccagaggcggttgcggaacacgctattggtatgatgatgacccttaatcgtcgcatccatcgtgcatatcagcgcacgcgcgatgctaacttcagtttagaaggattaacgggatttacaatgtacgggaagaccgctggcgtgattggcaccggaaaaatcggtgtggcaatgctgcgtatcttgaaggggtttggcatgcgtttgttagcatttgatccctatccaagtgccgcggccctggaactgggagtggaatatgttgatttgccaactttgtttagcgagtccgatgttatctcattgcattgtccacttactccggagaattatcatttattgaatgaagccgccttcgaacaaatgaaaaatggagtgatgatcgtaaatacaagtcgtggcgcgttgatcgattcgcaggcagcgatcgaagcgttaaaaaatcaaaagattggatcactgggcatggatgtctatgaaaacgagcgcgaccttttctttgaagacaaaagtaatgatgttatccaagatgatgtatttcgccgtctgtcggcatgccataatgtactttttacgggtcaccaagcattccttactgccgaggctctgactagcatttcacaaaccactcttcagaatctttcaaatcttgagaaaggtgagacgtgccccaatgaattggtttaagcatgctaatcagccgtggaattcggtctcaggaggtacgcatggcatggatgaccgatggtagtgtgggctctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgccagctgcattaatgaagaaatcatgctggaagaataacagctcactcaaaggcggtagtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaatt

SEQ ID NO: 2 - DNA, 5496 bp, Artificial Sequence
  Description of Artificial Sequence Synthetic polynucleotide
caatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcgctcccggagacggtcacagcttgtcaaaaaaaaaccccgcttcggcggggtttttttttggtacctcaagttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggaggtatacatatgaaacttgctgtatatagtaccaaacagtacgacaaaaagtaccttcaacaggtcaacgagagctttggtttcgaacttgaatttttcgactttttacttaccgagaaaacggcaaaaacggcgaacggatgtgaagcggtttgcattttcgtcaacgacgacggcagccgccctgttttagaagagttaaagaaacatggagttaaatacatcgcattacgttgtgcaggtttcaacaacgttgatctggatgctgcgaaggaactgggattgaaagttgtgcgcgtgcccgcttatgacccagaggcggttgcggaacacgctattggtatgatgatgacccttaatcgtcgcatccatcgtgcatatcagcgcacgcgcgatgctaacttcagtttagaaggattaacgggatttacaatgtacgggaagaccgctggcgtgattggcaccggaaaaatcggtgtggcaatgctgcgtatcttgaaggggtttggcatgcgtttgttagcatttgatccctatccaagtgccgcggccctggaactgggagtggaatatgttgatttgccaactttgtttagcgagtccgatgttatctcattgcattgtccacttactccggagaattatcatttattgaatgaagccgccttcgaacaaatgaaaaatggagtgatgatcgtaaatacaagtcgtggcgcgttgatcgattcgcaggcagcgatcgaagcgttaaaaaatcaaaagattggatcactgggcatggatgtctatgaaaacgagcgcgaccttttctttgaagacaaaagtaatgatgttatccaagatgatgtatttcgccgtctgtcggcatgccataatgtactttttacgggtcaccaagcattccttactgccgaggctctgactagcatttcacaaaccactcttcagaatctttcaaatcttgagaaaggtgagacgtgccccaatgaattggtttaagcatgctaatcagccgtggaattcggtctcaggaggtacgcatggcatggatgaccgatggtagtgtgggctctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgccagctgcattaatgaagaaatcatgctggaagaataacagctcactcaaaggcggtagtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttac

SEQ ID NO: 3 - DNA, 987 bp, Artificial Sequence
  Description of Artificial Sequence Synthetic polynucleotide
atgaaacttgctgtatatagtaccaaacagtacgacaaaaagtaccttcaacaggtcaacgagagctttggtttcgaacttgaatttttcgactttttacttaccgagaaaacggcaaaaacggcgaacggatgtgaagcggtttgcattttcgtcaacgacgacggcagccgccctgttttagaagagttaaagaaacatggagttaaatacatcgcattacgttgtgcaggtttcaacaacgttgatctggatgctgcgaaggaactgggattgaaagttgtgcgcgtgcccgcttatgacccagaggcggttgcggaacacgctattggtatgatgatgacccttaatcgtcgcatccatcgtgcatatcagcgcacgcgcgatgctaacttcagtttagaaggattaacgggatttacaatgtacgggaagaccgctggcgtgattggcaccggaaaaatcggtgtggcaatgctgcgtatcttgaaggggtttggcatgcgtttgttagcatttgatccctatccaagtgccgcggccctggaactgggagtggaatatgttgatttgccaactttgtttagcgagtccgatgttatctcattgcattgtccacttactccggagaattatcatttattgaatgaagccgccttcgaacaaatgaaaaatggagtgatgatcgtaaatacaagtcgtggcgcgttgatcgattcgcaggcagcgatcgaagcgttaaaaaatcaaaagattggatcactgggcatggatgtctatgaaaacgagcgcgaccttttctttgaagacaaaagtaatgatgttatccaagatgatgtatttcgccgtctgtcggcatgccataatgtactttttacgggtcaccaagcattccttactgccgaggctctgactagcatttcacaaaccactcttcagaatctttcaaatcttgagaaaggtgagacgtgccccaatgaattggtt

SEQ ID NO: 4 - DNA, 937 bp, Artificial Sequence
  Description of Artificial Sequence Synthetic polynucleotide
atgaaaaaggtcaatcgtattgcagtggttggaacgggtgcagttggtacaagttactgctacgccatgattaatcagggtgttgcagaagagcttgttttaatcgatattaacgaagcaaaagcagaaggggaagccatggacctgaaccacggcctgccatttgcgcctacgccgacccgcgtttggaaaggcgattattccgattgcggcactgccgatcttgttgtcattacggcaggttccccgcaaaaaccgggcgaaacaaggcttgatcttgtttccaaaaacgcaaaaatttttaaaggcatgattaagagcatcatggacagcggctttaacgggatttttcttgttgccagcaacccggttgacattttgacatatgtaacttggaaagagtccggcctgccgaaagaacatgttatcggttcgggcacagtgcttgactccgcgcgtctccgcaactctttgagcgcccaatttggaattgacccgcgcaatgtgcatgctgcgattatcggcgaacacggcgatacggaacttccggtatggagccatacaaatatcggttacgatacgattgaaagctatctacaaaaaggaattattgacgaaaagacgttagatgacatttttgtcaatacgagagatgcggcttatcatattattgaacgaaaaggggccacattttacggcatcgggatgtccctgacccggattacaagggcaatcctgaacaatgaaaacagcgtattgacggtctctgcatttcttgaaggccaatacggaaacagcgatgtgtacgttggcgttccggccatcatcaatcgccagggcatccgtgaagtggttgaaatcaaactgaacgaaaaagaacaggaacagttcaatcattctgtaaaagtgctaaaagaaacgatggcacctgtattgt

SEQ ID NO: 5 - DNA, 2130 bp, Artificial Sequence
  Description of Artificial Sequence Synthetic polynucleotide
atgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcatcgcgaactaccacgctaacaccaaagacgctgaagtcgttctggtggaaggtctggtcccgacacgtaagcaccagtttgcccagtctctgaactacgaaatcgccaaaacgctgaacgcagaaatcgtcttcgttatgtctcagggcactgatactccggaacagttgaaagagcgtatcgaactgactcgcaacagcttcggcggtgcaaaaaacaccaatattaccggcgttatcgttaacaaactgaacgctccggttgatgagcagggtcgtacccgtccggatctgtccgagatttttgacgactccaccaaagcaaaagtgaacaacgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgccctgacgtgctggttgccgcttgcctggctgccatgaacggcgtagaaatcggtgccctgctgctgactggcggctacgaaatggacgcgcgcatttctaaactgtgcgaacgtgctttcgctactggcctgccggtatttatggtgaacaccaacacctggcagacttctcttagcctgcagagcttcaacctggaagttccggttgacgatcatgagcgtatcgaaaaagttcaggaatacgtggctaactacatcaacgctgactggatcgattctctgactgccacttctgagcgcagccgtcgtctgtctccgccagcgttccgttatcagctgactgaacttgcgcgcaaagcgggcaaacgtatcgttctgccggaaggtgacgaaccgcgtaccgttaaagcagccgctatctgtgctgaacgtggtatcgcaacttgcgtactgctgggtaatccggcagagatcaaccgtgttgcagcctctcagggtgtagaactgggtgcaggcattgaaatcgttgatccagaagtggttcgcgaaaactatgttggtcgtctggtcgaactgcgtaagaacaaaggcatgaccgaaaccgttgcccgcgaacagctggaagacaacgtggttctcggtacgctgatgctggaacaagatgaagttgatggtctggtttccggtgctgttcacaccaccgcaaacaccatccgtccgccgctgcagctgatcaaaactgcaccgggcagctccctggtatcttccgtgttcttcatgctgttgccggaacaggtttacgtttacggtgactgtgcgatcaacccggatccgaccgcagaacagctggcagaaatcgcgattcagtccgctgattccgctgcggccttcggtatcgaaccgcgcgttgctatgctctcctactccaccggtacttctggtgctggtagcgacgtagaaaaagttcgcgaagcaactcgtctggcgcaggaaaaacgtcctgatctgatgatcgacggtccgctgcagtacgacgctgcggtaatggctgacgttgcgaaatccaaagcaccgaactctccggttgcaggtcgcgctaccgtgttcatcttcccggatctgaacaccggtaacaccacctacaaagcggtacagcgttctgctgacctgatctctatcggaccgatgctgcagggtatgcgcaagccggttaacgacctgtcccgtggcgcactggttgatgatatcgtctacaccatcgcgctgactgcgattcagtctgcacagcagcagtaa

SEQ ID NO: 6 - DNA, 2283 bp, Artificial Sequence
  Description of Artificial Sequence Synthetic polynucleotide
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggttacatcaacaagcagcttgagaaaatcgttggtctgcagactgaagctccgctgaaacgtgctcttatcccgttcggtggtatcaaaatgatcgaaggttcctgcaaagcgtacaaccgcgaactggacccgatgatcaaaaaaatcttcactgaataccgtaaaactcacaaccagggcgtgttcgacgtttacactccggacatcctgcgttgccgtaaatccggtgttctgaccggtctgccagatgcttatggccgtggtcgtatcatcggtgactaccgtcgcgttgcgctgtacggtatcgactacctgatgaaagacaaacttgcacagttcacctctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaacgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaactggtaacaccccagacggtcgtcgtgctggcgcgccgttcggaccgggtgctaacccgatgcacggtcgtgaccagaaaggtgctgtagcgtctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 7 - DNA, 1203 bp, Artificial Sequence
  Description of Artificial Sequence Synthetic polynucleotide
atgtcgagtaagttagtactggttctgaactgcggtagttcttcactgaaatttgccatcatcgatgcagtaaatggtgaagagtacctttctggtttagccgaatgtttccacctgcccgaagcacgtatcaaatggaaaatggacggcaataaacaggaagcggctttaggtgcaggcgccgctcacagcgaagcgctcaactttatcgttaatactattctggcacaaaaaccagaactgtctgcgcagctgactgctatcggtcaccgtatcgtacacggcggcgaaaagtataccagctccgtagtgatcgatgagtctgttattcagggtatcaaagatgcagcttcttttgcaccgctgcacaacccggctcacctgatcggtatcgaagaagctctgaaatctttcccacagctgaaagacaaaaacgttgctgtatttgacaccgcgttccaccagactatgccggaagagtcttacctctacgccctgccgtacaacctgtacaaagagcacggcatccgtcgttacggcgcgcacggcaccagccacttctatgtaacccaggaagcggcaaaaatgctgaacaaaccggtagaagaactgaacatcatcacctgccacctgggcaacggtggttccgtttctgctatccgcaacggtaaatgcgttgacacctctatgggcctgaccccgctggaaggtctggtcatgggtacccgttctggtgatatcgatccggcgatcatcttccacctgcacgacaccctgggcatgagcgttgacgcaatcaacaaactgctaaccaaagagtctggcctgctgggtctgaccgaagtgaccagcgactgccgctatgttgaagacaactacgcgacgaaagaagacgcgaagcgcgcaatggacgtttactgccaccgcctggccaaatacatcggtgcctacactgcgctgatggatggtcgtctggacgctgttgtattcaccggtggtatcggtgaaaatgccgcgatggttcgtgaactgtctctgggcaaactgggcgtgctgggctttgaagttgatcatgaacgcaacctggctgcacgtttcggcaaatctggtttcatcaacaaagaaggtacccgtcctgcggtggttatcccaaccaacgaagaactggttatcgcgcaagacgcgagccgcctgactgcctga

SEQ ID NO: 8 - DNA, 2676 bp, Artificial Sequence
  Description of Artificial Sequence Synthetic polynucleotide
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcttataaagatgaaaaaacctgtggtgttctgtctgaagacgacacttttggtaccatcactatcgctgaacccatcggtattatttgcggtatcgttccgaccactaacccgacttcaactgctatcttcaaatcgctgatcagcctgaagacccgtaacgccattatcttctccccgcacccgcgtgcaaaagatgcaaccaacaaagcggctgatatcgttctacaggctgctatcgctgccggtgctccgaaagatctgatcggctggatcgatcaaccttctgttgagctgtctaacgcactgatgcaccacccagacatcaacctgatcctcgcgactggtggtccgggcatggttaaagccgcatacagctccggtaaaccagctatcggcgtaggcgcgggcaacactccggttgttatcgatgaaactgctgatatcaaacgtgcagttgcatctgtactgatgtccaaaaccttcgacaacggtgtaatctgtgcttctgaacagtctgttgttgttgttgactctgtttatgacgcagtacgtgaacgtttcgcaacccacggcggctatctgttgcagggtaaagagctgaaagctgttcaggacgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgtgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggctcgtatcctgattaacaccccggcttctcagggtggtatcggtgacctgtataacttcaaactcgcaccttccctgactctgggttgtggttcctggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtattgaaagcagcaggcgttgaaactgaagtcttcttcgaagtagaagctgacccgaccctgagcatcgttcgtaaaggtgcagaactggcaaactccttcaaaccagacgtgattatcgcgctgggtggaggttccccgatggacgctgcgaagatcatgtgggttatgtacgaacatccggaaactcacttcgaagaactggcgctgcgctttatggatatccgtaaacgtatctacaagttcccgaaaatgggtgtgaaagcgaaaatgatcgctgtcaccaccacttctggtacaggttctgaagtcactccgtttgcggttgtaactgacgacactactggtcagaaatatccgctggcagactatgcactgaccccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaacgcgaatgacaacccgaccaagcagactgcattcagccagtatgaccgtccgcaggctcgccgtcgttatgctgaaattgctgaccacctgggtctgagcgcaccgggcgaccgtactgcagctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggcgaaactgcagcgaagaaagaagctgctccggctaaagctgagaaaaaagcgaaaaaatccgcttaa

SEQ ID NO: 9 - DNA, 1023 bp, Artificial Sequence
  Description of Artificial Sequence Synthetic polynucleotide
atgtgcaagaagacttccggcaacagatttcattttgcattccaaagttcagaggtagtcatgattaagaaaatcggtgtgttgacaagcggcggtgatgcgccaggcatgaacgccgcaattcgcggggttgttcgttctgcgctgacagaaggtctggaagtaatgggcatttatgacggctatctgggtctgtatgaagaccgtatggtacagctagaccgttacagcgtttctgacatgatcaaccgtggtggtacgttcctcggttctgcgcgtttcccggaattccgcgacgagaacatccgcgccgtggctatcgaaaacctgaaaaaacgtgggatcgacgcgctggtggttatcggcggtgacggttcctacatgggtgcaatgcgtctgaccgaaatgggcttcccgtgcatcggcctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggcgatctgacgttggctgcggctattgccggcggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgagctgctgctggcaggttacggtggtcgttgcgtaggtatccagaacgaacagctggttcaccacgacatcatcgacgctatcgaaaacatgaagcgtccgttcaaaggcgactggctggactgcgcgaaaaaactgtattaa

SEQ ID NO: 10 - DNA, 1809 bp, Artificial Sequence
  Description of Artificial Sequence Synthetic polynucleotide
gtgcaaacctttcaagccgatcttgccattgtaggcgccggtggcgcgggattacgtgctgcaattgctgccgcgcaggcaaatccaaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtgggggtgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctaccggcggtgcgggtcgcgtttatcgttacaacaccaacggcggcatcgttaccggtgacggtatgggtatggcgctaagccacggcgttccgctgcgtgacatggaattcgttcagtatcacccaaccggtctaccaggttccggtatcctgatgaccgaaggctgccgcggtgaaggtggtattctggtcaacaaaaatggctaccgttatctgcaagattacggcatgggcccggaaactccgctgggcgagccgaaaaacaaatatatggaactgggtccacgcgacaaagtttctcaggccttctggcacgaatggcgtaaaggcaacaccatctccacgccacgtggcgatgtggtttacctcgacctgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcgaaccgtctgggctccaactcgctggcggaactggtggtcttcggtcgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggctaagatccgcgacgaaatgggcatggcaatggaagaaggttgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgagctgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgtgtttacggtggcgaagcggatgcagccgataaggcggaagcagccaataagaaggagaaggcgaatggctga

SEQ ID NO: 11 - DNA, 1719 bp, Artificial Sequence
  Description of Artificial Sequence Synthetic polynucleotide
atgaaacaaacggttgcagcttatatcgccaaaacactcgaatcggcaggcgtgaaacgcatctggggagtcacgggcgactctctgaacggtcttagtgacagtcttaatcgcatgggcaccatcgagtggatgtccacccggcatgaagaagtggcggcgtttgccgctggcgctgaagcacaacttagcggagaactggcggtctgtgccggatcgtgcggtcccggcaacctgcacttaatcaacggcctgttcgattgccaccgcaatcacgttccggtactggcgattgccgctcatattccctccagcgaaattggcagcggctatttccaggaaacccacccacaagagctattccgcgaatgtagtcactattgcgagcttgtttccagcccagagcagatcccacaagtgctggcaattgctatgcgcaaagcggtgcttaaccgtggcgtttccgttgttgtgttaccgggcgacgtggcgttaaaacctgcgccagaaggggcaactacccactggtatcatgcgccacagccggtagtaacaccggaagaagaagagttacgcaaactggcgcaactgctgcgttattccagcaatatcgccctgatgtgtggcagcggctgtgcgggggcgcataaagagttagttgagtttgccgggaaaattaaagcgcctatagttcatgccctgcgcggtaaagagcatgtcgaatacgataatccgtatgatgtcggaatgacgggattaatcggcttctcgtcaggtttccataccatgatgaatgccgatacgttagtgctgctcggcacgcaatttccctaccgcgcgttctacccgaccgatgccaaaattattcagattgatatcaacccagccagcatcggcgcgcatagcaaggtagatatggcgctggtcggcgatatcaaatcaaccctgcgggcattgctgccactggtggaagaaaaaaccgatcgcaagtttctggataaagcgctggaagattaccgcgacgcccgcaaagggctggatgatttagctaaaccgagcgagaaagccattcacccgcaatatctggcgcagcaaattagtcattttgccgccgatgacgccatctttacctgtgacgttggtacgccaacggtgtgggcggcacgttatctgaaaatgaacggcaagcgtcgtctgttaggttcgtttaaccacggttcgatggctaacgccatgccgcaggcgctgggtgcgcaggcaaccgagccggaacgtcaggtggtcgccatgtgcggcgatggcggtttcagtatgttgatgggcgatttcctctcagtaatgcagatgaaattgccagtgaaaattatcgtctttaataacagcgtgctgggctttgtggcgatggagatgaaagccggaggctacctgacagacggtactgagctgcacgacaccaactttgcccgaattgccgaagcctgcggcattacgggtattcgtgtagaaaaagcgtctgaaatcgatgaagctctgcaacgcgccttctccatcgacggtccggtgttggtggatgtggtggtcgccaaagaagaattagccattccaccgcagatcaaacttgaacaggccaaaggtttcagcctgtatatgctgcgcgcaatcatcagcgggcgcggtgatgaagtgatcgaactggcgaaaacgaactggctaaggtaa

SEQ ID NO: 12 - DNA, 2379 bp, Artificial Sequence
  Description of Artificial Sequence Synthetic polynucleotide
atgtccaacaatggctcgtcaccgctggtgctttggtataaccaactcggcatgaatgatgtagacagggttgggggcaaaaatgcctccctgggtgaaatgattactaacctttccggaatgggtgtttccgttccgaatggtttcgccacaaccgccgacgcgtttaaccagtttctggaccaaagcggcgtaaaccagcgcatttatgaactgctggataaaacggatattgacgatgttacccagcttgcgaaagcgggcgcgcaaatccgccagtggattatcgacactcccttccagcctgagctggaaaacgccatccgcgaagcctatgcacagctttccgccgatgacgaaaacgcttcgtttgcggtgcgttcctccgctactgcagaagatatgccggacgcttcttttgccggtcagcaggaaactttcctcaacgttcagggttttgacgccgttctcgtggcagtgaagcatgtatttgcttctctgtttaacgatcgcgccatctcttatcgtgtgcaccagggttacgaccatcgtggcgtagcgctctccgccggtgttcagcggatggtgcgctccgacctcgcatcttctggcgtgatgttctccattgataccgaatctggctttgaccaggtggtgtttatcacttccgcatggggccttggtgaaatggtcgtgcagggtgcggttaacccggatgagttttatgtgcataaaccgacacttgcggcgaatcgcccggctattgtgcgccgcaccatggggtcgaaaaaaatccgcatggtttacgcgccgacccaggagcacggcaagcaggttaaaatcgaagacgtaccgcaggaacagcgtgacatcttctcgctgaccaacgaagaagtgcaggaactggcaaaacaggccgtacaaattgagaaacactacggtcgcccgatggatattgagtgggcgaaagatggccacaccggcaaactgttcattgtgcaggcgcgtccggaaaccgtgcgctcacgcggtcaggtcatggagcgttatacgctgcattcacagggtaagattatcgccgaaggccgtgctatcggtcatcgcatcggcgcgggtccggtgaaagtcatccatgacatcagcgaaatgaaccgcatcgaacctggcgacgtgctggttactgacatgaccgacccggactgggaaccgatcatgaagaaagcatctgccatcgtcaccaaccgtggcggtcgtacctgtcacgcggcgatcatcgctcgtgaactcggcattccggcggtagtgggctgtggtgatgcaacagaacggatgaaagacggcgagaacgtcactgtttcttgtgccgaaggtgataccggttacgtctatgcggagttgctggaatttagcgtgaaaagctccagcgtagaaacgatgccggacctgccgttgaaggtgatgatgaacgtcggtaacccggaccgtgctttcgacttcgcctgcctgccgaacgaaggcgtgggccttgcgcgtctggaatttatcatcaaccgtatgattggcgtccacccacgcgcactgcttgagtttgacgatcaggaaccgcagttgcaaaacgaaatccgcgagatgatgaaaggttttgattctccgcgtgaattttacgttggtcgtctgactgaagggatcgcgacgctgggtgccgcgttttatccgaagcgcgtcattgtccgtctctctgattttaaatcgaacgaatatgccaatctggtcggtggtgagcgttacgagccagatgaagagaacccgatgctcggcttccgtggcgcgggccgctatgtttccgacagcttccgcgactgcttcgcgctggagtgtgaagcagtgaaacgtgtgcgcaacgacatggggctgactaacgttgagatcatgatcccgttcgtgcgtaccgttgatcaggcgaaagcggtggttgaggaactggcgcatcaggggctgaaacgtggtgagaacgggctgaaaatcatcatgatgtgtgaaattccgtccaacgccttgctggcagagcagttcctggaatatttcgacggcttctcaattggctcaaacgacatgacgcagctggcgctcggtctggatcgtgactccggcgtggtgtctgaactgttcgatgagcgcaacgatgcggtgaaagcactgctgtcgatggcgattcgtgccgcgaagaaacagggcaaatatgtcgggatttgcggtcagggtccatccgaccacgaagactttgctgcatggttgatggaagaggggatcgatagcctgtctctgaacccggacaccgtggtgcaaacctggttaagcctggctgaactgaagaaataa

SEQ ID NO: 13 - DNA, 1716 bp, Artificial Sequence
  Description of Artificial Sequence Synthetic polynucleotide
atgtcttccatgacaacaactgataataaagcctttttgaatgaacttgcccgtctggtcggtcattcacacctgctcaccgatcccgcaaaaacggcccgctatcgcaagggcttccgttctggtcagggcgacgcgcttgctgtcgttttccctggctcactactagaattgtggcgggtgctgaaagcctgcgtcaccgctgacaaaattattctgatgcaggctgccaatacaggcctgaccgaaggatcgacgccaaacggtaacgattatgatcgcgatatcgtgatcatcagcaccctgcgtctcgacaagctgcacgttctcggcaagggcgaacaagtgctggcctatccgggcaccacgctctattcactggaaaaagccctcaaaccgctgggacgcgaaccgcactcagtgattggatcatcgtgtataggcgcatcggtcatcggcggtatttgtaacaactcgggcggctcgctggtgcaacgtggcccggcgtataccgaaatgtcattattcgcgcgtataaatgaagacggcaaactgacgctggtgaaccatctggggattgatctgggcgaaacgccggagcagatccttagcaagctggatgacgatcgcatcaaagatgacgatgtgcgtcacgatggtcgtcacgcccacgattatgactatgtccaccgcgttcgtgatattgaagccgacacgcccgcacgttataacgccgatccggatcggttatttgaatcttctggttgcgcaggtaagctggccgtctttgcggtacgtcttgataccttcgaagcggaaaaaaatcagcaggtgttttatatcggcaccaaccagccggaagtgctgaccgaaatccgccgtcatattctggctaatttcgaaaatctgccggttgccggggaatatatgcaccgggatatctacgatattgcggaaaaatacggcaaagacaccttcctgatgattgataagttaggcaccgacaagatgccgttcttctttaatctcaagggacgcaccgatgcgatgctggagaaagtgaaattcttccgtccgcattttaccgaccgtgcaatgcaaaaattcggtcacctgttccccagccatttaccgccgcgcatgaaaaactggcgcgataaatacgagcatcatctgctgttaaaaatggcgggcgatggcgtcggtgaagccaaatcgtggctagtggattatttcaaacaggccgagggcgatttctttgtctgtacgccggaggaaggcagcaaagcgtttttacaccgtttcgccgctgcgggcgcagcaattcgttatcaggctgtgcattccgatgaagtcgaagacattctggcgctggatatcgctctgcggcgtaacgacaccgaatggtatgagcatttaccgccggagatcgacagccagctggtgcacaagctctattatggccattttatgtgctatgtcttccatcaggattacatcgtgaaaaaaggcgtggatgtgcatgtgttgaaagaacagatgctggaactgctacagcagcgcggcgcgcaataccctgccgagcataacgtcggtcatttgtataaagcaccggaaacgttgcagaagttttatcgcgagaacgatccgaccaacagtatgaatccggggatcggtaaaaccagtaagcggaaaaactggcaggaagtggagtaa

SEQ ID NO: 14 - DNA, 1191 bp, Artificial Sequence
  Description of Artificial Sequence Synthetic polynucleotide
atgattatttccgcagccagcgattatcgcgccgcagcgcaacgcattctgccgccgttcctgttccactatatggatgggggggcatattctgaatacacgctgcgccgcaacgtggaagatttgtcagaagtggcgctgcgccagcgtattctgaaaaacatgtctgacttaagcctggaaacgacgctgtttaatgagaaattgtcgatgccggtggcgctaggtccggtaggtttgtgtggcatgtatgcgcgacgcggcgaagttcaggctgccaaagcagcagatgcgcatggcattccgtttactctctcgacggtttccgtttgcccgattgaagaagtggctccggctatcaaacgtccgatgtggttccagctttatgtgctgcgcgatcgcggctttatgcgtaacgccctggagcgagcaaaagccgcgggttgttcgacgctggttttcaccgtggatatgccaacgccgggagcgcgttatcgtgatgcgcattctgggatgagcggcccgaacgcggcaatgcgccgctacttgcaggcggtgacgcatccgcaatgggcgtgggatgtgggcctgaacggtcgtccgcatgatttaggtaatatctcggcttacctcggcaaaccaaccggactggaagattacatcggctggctggggaataacttcgatccgtccatctcatggaaagaccttgagtggatccgcgatttctgggatggcccgatggtgatcaaagggatcctcgatccggaagatgcgcgcgatgcagtacgttttggtgctgatggaattgtggtttctaaccacggtggccgccagttagatggcgtactctcttctgctcgtgcactgcctgctattgcggatgcggtgaaaggtgatatcgccattctggcggatagcggaatacgtaacgggcttgatgtcgtgcgtatgattgcgctcggtgccgacaccgtactgctgggtcgtgctttcctgtatgcactggcaacagcgggccaggcgggtgtagctaatctgctaaatctgatcgaaaaagagatgaaagtggcgatgacgctgactggcgcgaaatcgattagcgaaattacgcaagattcgctggtgcaggggctgggtaaagagttgcctgcggcactggctccaatggcgaaagggaatgcagcttaa

SEQ ID NO: 15 - protein, 329 aa, Artificial Sequence
  Description of Artificial Sequence Synthetic polypeptide
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 16 - protein, 312 aa, Artificial Sequence
  Description of Artificial Sequence Synthetic polypeptide
MKKVNRIAVVGTGAVGTSYCYAMINQGVAEELVLIDINEAKAEGEAMDLNHGLPFAPTPTRVWKGDYSDCGTADLVVITAGSPQKPGETRLDLVAKNAKIFKGMIKSIMDSGFNGIFLVASNPVDILTYVTWKESGLPKEHVIGSGTVLDSARLRNSLSAHFGIDPRNVHAAIIGEHGDTELPVWSHTTIGYDTIESYLQKGTIDQKTLDDIFVNTRDAAYHIIERKGATFYGIGMSLTRITRAILNNENSVLTVSAFLEGQYGNSDVYIGVPAVINRQGVREVVEIELNDKEQEQFSHSVKVLKETMAPVL

SEQ ID NO: 17 - protein, 709 aa, Artificial Sequence
  Description of Artificial Sequence Synthetic polypeptide
MLIPTGTSVGLTSVSLGVIRAMERKGVRLSVFKPIAQPRTGGDAPDQTTTIVRANSSTTTAAEPLKMSYVEGLLSSNQKDVLMEEIIANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSTKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIDSLTATSERSRRLSPPAFRYQLTELARKAGKRIVLPEGDEPRTVKAAAICAERGIATCVLLGNPAEINRVAASQGVELGAGIEIVDPEVVRENYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQSAQQQ

SEQ ID NO: 18 - protein, 760 aa, Artificial Sequence
  Description of Artificial Sequence Synthetic polypeptide
MSELNEKLATAWEGFTKGDWQNEVNVRDFIQKNYTPYEGDESFLAGATEATTTLWDKVMEGVKLENRTHAPVDFDTAVASTITSHDAGYINKQLEKIVGLQTEAPLKRALIPFGGIKMIEGSCKAYNRELDPMIKKIFTEYRKTHNQGVFDVYTPDILRCRKSGVLTGLPDAYGRGRIIGDYRRVALYGIDYLMKDKLAQFTSLQADLENGVNLEQTIRLREEIAEQHRALGQMKEMAAKYGYDISGPATNAQEAIQWTYFGYLAAVKSQNGAAMSFGRTSTFLDVYIERDLKAGKITEQEAQEMVDHLVMKLRMVRFLRTPEYDELFSGDPIWATESIGGMGLDGRTLVTKNSFRFLNTLYTMGPSPEPNMTILWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNYDEVMERMDHFMDWLAKQYITALNIIHYMHDKYSYEASLMALHDRDVIRTMACGIAGLSVAADSLSAIKYAKVKPIRDEDGLAIDFEIEGEYPQFGNNDPRVDDLAVDLVERFMKKIQKLHTYRDAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMENPEKYPQLTIRVSGYAVRFNSLTKEQQQDVITRTFTQSM

SEQ ID NO: 19 - protein, 400 aa, Artificial Sequence
  Description of Artificial Sequence Synthetic polypeptide
MSSKLVLVLNCGSSSLKFAIIDAVNGEEYLSGLAECFHLPEARIKWKMDGNKQEAALGAGAAHSEALNFIVNTILAQKPELSAQLTAIGHRIVHGGEKYTSSVVIDESVIQGIKDAASFAPLHNPAHLIGIEEALKSFPQLKDKNVAVFDTAFHQTMPEESYLYALPYNLYKEHGIRRYGAHGTSHFYVTQEAAKMLNKPVEELNIITCHLGNGGSVSAIRNGKCVDTSMGLTPLEGLVMGTRSGDIDPAIIFHLHDTLGMSVDAINKLLTKESGLLGLTEVTSDCRYVEDNYATKEDAKRAMDVYCHRLAKYIGAYTALMDGRLDAVVFTGGIGENAAMVRELSLGKLGVLGFEVDHERNLAARFGKSGFINKEGTRPAVVIPTNEELVIAQDASRLTA

SEQ ID NO: 20 - protein, 891 aa, Artificial Sequence
  Description of Artificial Sequence Synthetic polypeptide
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDTTGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 21 - protein, 340 aa, Artificial Sequence
  Description of Artificial Sequence Synthetic polypeptide
MCKKTSGNRFHFAFQSSEVVMIKKIGVLTSGGDAPGMNAAIRGVVRSALTEGLEVMGIYDGYLGLYEDRMVQLDRYSVSDMINRGGTFLGSARFPEFRDENIRAVAIENLKKRGIDALVVIGGDGSYMGAMRLTEMGFPCIGLPGTIDNDIKGTDYTIGFFTALSTVVEAIDRLRDTSSSHQRISVVEVMGRYCGDLTLAAAIAGGCEFVVVPEVEFSREDLVNEIKAGIAKGKKHAIVAITEHMCDVDELAHFIEKETGRETRATVLGHIQRGGSPVPYDRILASRMGAYAIELLLAGYGGRCVGIQNEQLVHHDIIDAIENMKRPFKGDWLDCAKKLY

SEQ ID NO: 22 - protein, 602 aa, Artificial Sequence
  Description of Artificial Sequence Synthetic polypeptide
VQTFQADLAIVGAGGAGLRAAIAAAQANPNAKIALISKVYPMRSHTVAAEGGSAAVAQDHDSFEYHFHDTVAGGDWLCEQDVVDYFVHHCPTEMTQLELWGCPWSRRPDGSVNVRRFGGMKIERTWFAADKTGFHMLHTLFQTSLQFPQIQRFDEHFVLDILVDDGHVRGLVAMNMMEGTLVQIRANAVVMATGGAGRVYRYNTNGGIVTGDGMGMALSHGVPLRDMEFVQYHPTGLPGSGILMTEGCRGEGGILVNKNGYRYLQDYGMGPETPLGEPKNKYMELGPRDKVSQAFWHEWRKGNTISTPRGDVVYLDLRHLGEKKLHERLPFICELAKAYVGVDPVKEPIPVRPTAHYTMGGIETDQNCETRIKGLFAVGECSSVGLHGANRLGSNSLAELVVFGRLAGEQATERAATAGNGNEAAIEAQAAGVEQRLKDLVNQDGGENWAKIRDEMGMAMEEGCGIYRTPELMQKTIDKLAELQERFKRVRITDTSSVFNTDLLYTIELGHGLNVAECMAHSAMARKESRGAHQRLDEGCTERDDVNFLKHTLAFRDADGTTRLEYSDVKITTLPPAKRVYGGEADAADKAEAANKKEKANG

SEQ ID NO: 23 - protein, 572 aa, Artificial Sequence
  Description of Artificial Sequence Synthetic polypeptide
MKQTVAAYIAKTLESAGVKRIWGVTGDSLNGLSDSLNRMGTIEWMSTRHEEVAAFAAGAEAQLSGELAVCAGSCGPGNLHLINGLFDCHRNHVPVLAIAAHIPSSEIGSGYFQETHPQELFRECSHYCELVSSPEQIPQVLAIAMRKAVLNRGVSVVVLPGDVALKPAPEGATTHWYHAPQPVVTPEEEELRKLAQLLRYSSNIALMCGSGCAGAHKELVEFAGKIKAPIVHALRGKEHVEYDNPYDVGMTGLIGFSSGFHTMMNADTLVLLGTQFPYRAFYPTDAKIIQIDINPASIGAHSKVDMALVGDIKSTLRALLPLVEEKTDRKFLDKALEDYRDARKGLDDLAKPSEKAIHPQYLAQQISHFAADDAIFTCDVGTPTVWAARYLKMNGKRRLLGSFNHGSMANAMPQALGAQATEPERQVVAMCGDGGFSMLMGDFLSVMQMKLPVKIIVFNNSVLGFVAMEMKAGGYLTDGTELHDTNFARIAEACGITGIRVEKASEIDEALQRAFSIDGPVLVDVVVAKEELAIPPQIKLEQAKGFSLYMLRAIISGRGDEVIELAKTNWLR

SEQ ID NO: 24 - protein, 792 aa, Artificial Sequence
  Description of Artificial Sequence Synthetic polypeptide
MSNNGSSPLVLWYNQLGMNDVDRVGGKNASLGEMITNLSGMGVSVPNGFATTADAFNQFLDQSGVNQRIYELLDKTDIDDVTQLAKAGAQIRQWIIDTPFQPELENAIREAYAQLSADDENASFAVRSSATAEDMPDASFAGQQETFLNVQGFDAVLVAVKHVFASLFNDRAISYRVHQGYDHRGVALSAGVQRMVRSDLASSGVMFSIDTESGFDQVVFITSAWGLGEMVVQGAVNPDEFYVHKPTLAANRPAIVRRTMGSKKIRMVYAPTQEHGKQVKIEDVPQEQRDIFSLTNEEVQELAKQAVQIEKHYGRPMDIEWAKDGHTGKLFIVQARPETVRSRGQVMERYTLHSQGKIIAEGRAIGHRIGAGPVKVIHDISEMNRIEPGDVLVTDMTDPDWEPIMKKASAIVTNRGGRTCHAAIIARELGIPAVVGCGDATERMKDGENVTVSCAEGDTGYVYAELLEFSVKSSSVETMPDLPLKVMMNVGNPDRAFDFACLPNEGVGLARLEFIINRMIGVHPRALLEFDDQEPQLQNEIREMMKGFDSPREFYVGRLTEGIATLGAAFYPKRVIVRLSDFKSNEYANLVGGERYEPDEENPMLGFRGAGRYVSDSFRDCFALECEAVKRVRNDMGLTNVEIMIPFVRTVDQAKAVVEELAHQGLKRGENGLKIIMMCEIPSNALLAEQFLEYFDGFSIGSNDMTQLALGLDRDSGVVSELFDERNDAVKALLSMAIRAAKKQGKYVGICGQGPSDHEDFAAWLMEEGIDSLSLNPDTVVQTWLSLAELKK

SEQ ID NO: 25 - protein, 571 aa, Artificial Sequence
  Description of Artificial Sequence Synthetic polypeptide
MSSMTTTDNKAFLNELARLVGHSHLLTDPAKTARYRKGFRSGQGDALAVVFPGSLLELWRVLKACVTADKIILMQAANTGLTEGSTPNGNDYDRDIVIISTLRLDKLHVLGKGEQVLAYPGTTLYSLEKALKPLGREPHSVIGSSCIGASVIGGICNNSGGSLVQRGPAYTEMSLFARINEDGKLTLVNHLGIDLGETPEQILSKLDDDRIKDDDVRHDGRHAHDYDYVHRVRDIEADTPARYNADPDRLFESSGCAGKLAVFAVRLDTFEAEKNQQVFYIGTNQPEVLTEIRRHILANFENLPVAGEYMHRDIYDIAEKYGKDTFLMIDKLGTDKMPFFFNLKGRTDAMLEKVKFFRPHFTDRAMQKFGHLFPSHLPPRMKNWRDKYEHHLLLKMAGDGVGEAKSWLVDYFKQAEGDFFVCTPEEGSKAFLHRFAAAGAAIRYQAVHSDEVEDILALDIALRRNDTEWYEHLPPEIDSQLVHKLYYGHFMCYVFHQDYIVKKGVDVHVLKEQMLELLQQRGAQYPAEHNVGHLYKAPETLQKFYRENDPTNSMNPGIGKTSKRKNWQEVE

SEQ ID NO: 26 - protein, 396 aa, Artificial Sequence
  Description of Artificial Sequence Synthetic polypeptide
MIISAASDYRAAAQRILPPFLFHYMDGGAYSEYTLRRNVEDLSEVALRQRILKNMSDLSLETTLFNEKLSMPVALGPVGLCGMYARRGEVQAAKAADAHGIPFTLSTVSVCPIEEVAPAIKRPMWFQLYVLRDRGFMRNALERAKAAGCSTLVFTVDMPTPGARYRDAHSGMSGPNAAMRRYLQAVTHPQWAWDVGLNGRPHDLGNISAYLGKPTGLEDYIGWLGNNFDPSISWKDLEWIRDFWDGPMVIKGILDPEDARDAVRFGADGIVVSNHGGRQLDGVLSSARALPAIADAVKGDIAILADSGIRNGLDVVRMIALGADTVLLGRAFLYALATAGQAGVANLLNLIEKEMKVAMTLTGAKSISEITQDSLVQGLGKELPAALAPMAKGNAA

SEQ ID NO: 27 - DNA, 169 bp, Unknown
  Description of Unknown FNR responsive promoter
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaacccgc

SEQ ID NO: 28 - DNA, 714 bp, Unknown
  Description of Unknown PcI857 promoter sequence
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcat

SEQ ID NO: 29 - protein, 21 aa, Artificial Sequence
  Description of Artificial Sequence Synthetic peptide
MEVGWYRPPFSRVVHLYRNGK

SEQ ID NO: 30 - DNA, 459 bp, Escherichia coli
atggaactgacgactcgcactttaccttcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggacccactaaacgccgtgccgcacgaccctgacgtgaaagccttgctgcgtctggcgacggtatggaacattccggttgccaccaacgtggcaacggcagacttcattatccagtcgccgcatttcaacgacgcggtcgatattctgatccccgattatcagcgttatctcgcggaccgtctgaagtaa

SEQ ID NO: 31 - protein, 152 aa, Escherichia coli
MELTTRTLPSRKHIALVAHDHCKQMLMSWVERHQPLLEQHVLYATGTTGNLISRATGMNVNAMLSGPMGGDQQVGALISEGKIDVLIFFWDPLNAVPHDPDVKALLRLATVWNIPVATNVATADFIIQSPHFNDAVDILIPDYQRYLADRLK

SEQ ID NO: 32 - DNA, 735 bp, Escherichia coli
atggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggctcctgcggcatgatggttaacaacgtgccaaaactggcatgtaaaaccttcctgcgtgattacaccgacggtatgaaggttgaagcgttagctaacttcccgattgaacgcgatctggtggtcgatatgactcacttcatcgaaagtctggaagcgatcaaaccgtacatcatcggcaactcccgcaccgcggatcagggtactaacatccagaccccggcgcagatggcgaagtatcaccagttctccggttgcatcaactgtggtctgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaa

SEQ ID NO: 33 - protein, 244 aa, Escherichia coli
MAEMKNLKIEVVRYNPEVDTAPHSAFYEVPYDATTSLLDALGYIKDNLAPDLSYRWSCRMAICGSCGMMVNNVPKLACKTFLRDYTDGMKVEALANFPIERDLVVDMTHFIESLEAIKPYIIGNSRTADQGTNIQTPAQMAKYHQFSGCINCGLCYAACPQFGLNPEFIGPAAITLAHRYNEDSRDHGKKERMAQLNSQNGVWSCTFVGYCSEVCPKHVDPAAAIQQGKVESSKDFLIATLKPR

SEQ ID NO: 34 - DNA, 396 bp, Escherichia coli
atgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagccctgctgcacaccaaaacctggtttgagctggcaccaaaagcagccaatatcattgtaaaagacgaaaaaatgggaccagagccaattatcaaaagtctctgggcggtaactgtggttgccaccatcgtaatcctgtttgttgccctgtactggtaa

SEQ ID NO: 35 - protein, 131 aa, Escherichia coli
MTTKRKPYVRPMTSTWWKKLPFYRFYMLREGTAVPAVWFSIELIFGLFALKNGPEAWAGFVDFLQNPVIVIINLITLAAALLHTKTWFELAPKAANIIVKDEKMGPEPIIKSLWAVTVVATIVILFVALYW